(19) United States
(12) Patent Application Publication
(10) Pub. No.: US 2022/0090158 A1
(43) Pub. Date: Mar. 24, 2022

(54) BIOSYNTHETIC PRODUCTION OF UDP-RHAMNOSE

(71) Applicant: Conagen Inc., Bedford, MA (US)

(72) Inventors: Guohong Mao, Burlington, MA (US); Phillip James Hunt, Tewksbury, MA (US); Michael James Batten, Westford, MA (US); Timothy Joseph Hanley, Waltham, MA (US); Oliver Yu, Lexington, MA (US)

(73) Assignee: Conagen Inc., Bedford, MA (US)

(21) Appl. No.: 17/487,532

(22) Filed: Sep. 28, 2021

Related U.S. Application Data
(63) Continuation of application No. PCT/US2020/025610, filed on Mar. 29, 2020.
(60) Provisional application No. 62/825,799, filed on Mar. 29, 2019.

(51) Int. Cl. C12P 19/56; C12N 9/04; C12N 15/52; C12N 9/90; C12N 9/88

(52) U.S. Cl. CPC C12P 19/56; C12N 9/0006; C12N 15/52; C12Y 101/01133; C12N 9/88; C12Y 501/03002; C12Y 402/01076; C12N 9/90

(57) ABSTRACT

The present disclosure relates to the biosynthesis of UDP-Rhamnose and recombinant polypeptides having enzymatic activity useful in the relevant biosynthetic pathways for producing UDP-Rhamnose. The present invention also provides a method for preparing a steviol glycoside composition comprising at least one rhamnose-containing steviol glycoside.

### CROSS-REFERENCE TO RELATED APPLICATIONS

This application claims priority to the U.S. Provisional Application Ser. No. 62/825,799, filed on Mar. 29, 2019, the disclosure of which is incorporated by reference herein in its entirety.

### FIELD OF INVENTION

The present disclosure generally relates to the biosynthesis of uridine diphosphate rhamnose (“UDP-rhamnose” or “UDPR” or “UDP-Rh”). More specifically, the present disclosure relates to biocatalytic processes for preparing UDP-rhamnose, which in turn can be used in the biosynthesis of rhamnose-containing steviol glycosides, as well as recombinant polypeptides having enzymatic activity useful in the relevant biosynthetic pathways for producing UDP-rhamnose and rhamnose-containing steviol glycosides.

### BACKGROUND OF THE INVENTION

Steviol glycosides are a class of compounds found in the leaves of Stevia rebaudiana plant that can be used as high intensity, low-calorie sweeteners. These naturally occurring steviol glycosides share the same basic diterpene structure (steviol backbone) but differ in the number and type of carbohydrate residues (e.g., glucose, rhamnose, and xylose residues) at the C13 and C19 positions of the steviol backbone. Interestingly, these variations in sugar ‘ornamentation’ of the basic steviol structure often dramatically and unpredictably affect the properties of the resulting steviol glycoside. The properties that are affected can include, without limitation, the overall taste profile, the presence and extent of any off-flavors, crystallization point, “mouth feel”, solubility and perceived sweetness among other differences. Steviol glycosides with known structures include stevioside, rebaudioside A (“Reb A”), rebaudioside B (“Reb B”), rebaudioside C (“Reb C”), rebaudioside D (“Reb D”), rebaudioside E (“Reb E”), rebaudioside F (“Reb F”), rebaudioside M (“Reb M”), rebaudioside J (“Reb J”), rebaudioside N (“Reb N”), and dulcoside A.

On a dry weight basis, stevioside, Reb A, Reb C, and dulcoside A account for approximately 9.1%, 3.8%, 0.6%, and 0.3%, respectively, of the total weight of all steviol glycosides found in wild type Stevia leaves. Other steviol glycosides such as Reb J and Reb N are present in significantly lower amounts. Extracts from the Stevia rebaudiana plant are commercially available. In such extracts, stevioside and Reb A typically are the primary components, while the other known steviol glycosides are present as minor or trace components. The actual content level of the various steviol glycosides in any given Stevia extract can vary depending on, for example, the climate and soil in which the Stevia plants are grown, the conditions under which the Stevia leaves are harvested, and the processes used to extract the desired steviol glycosides. To illustrate, the amount of Reb A in commercial preparations can vary from about 20% to more than about 90% by weight of the total steviol glycoside content, while the amount of Reb B, Reb C, and Reb D, respectively, can be about 1-2%, about 7-15%, and about 2% by weight of the total steviol glycoside content. In such extracts, Reb J and Reb N typically account for, individually, less than 0.5% by weight of the total steviol glycoside content.

As natural sweeteners, different steviol glycosides have different degrees of sweetness, mouth feel, and aftertastes. The sweetness of steviol glycosides is significantly higher than that of table sugar (i.e., sucrose). For example, stevioside itself is 100-150 times sweeter than sucrose but has a bitter aftertaste as noted in numerous taste tests, while Reb A and Reb E are 250-450 times sweeter than sucrose and the aftertaste profile is much better than stevioside. However, these steviol glycosides themselves still retain a noticeable aftertaste. Accordingly, the overall taste profile of any Stevia extract is profoundly affected by the relative content of the various steviol glycosides in the extract, which in turn may be affected by the source of the plant, the environmental factors (such as soil content and climate), and the extraction process. In particular, variations of the extraction conditions can lead to inconsistent compositions of the steviol glycosides in the Stevia extracts, such that the taste profile varies among different batches of extraction productions. The taste profile of Stevia extracts also can be affected by plant-derived or environment-derived contaminants (such as pigments, lipids, proteins, phenolics, and saccharides) that remain in the product after the extraction process. These contaminants typically have off-flavors undesirable for the use of the Stevia extract as a sweetener. In addition, the process of isolating individual or specific combinations of steviol glycosides that are not abundant in Stevia extracts can be cost- and resource-wise prohibitive.

Further, the extraction process from plants typically employs solid-liquid extraction techniques using solvents such as hexane, chloroform, and ethanol. Solvent extraction is an energy-intensive process, and can lead to problems relating to toxic waste disposal. Thus, new production methods are needed to both reduce the costs of steviol glycoside production as well as to lessen the environmental impact of large-scale cultivation and processing.

Accordingly, there is a need in the art for novel preparation methods of steviol glycosides, particularly rhamnose-containing steviol glycosides such as Reb J and Reb N, that can yield products with better and more consistent taste profiles. Given the fact that the biosynthetic pathways to such rhamnose-containing steviol glycosides often use UDP-rhamnose as one of the starting substrates, there is a need in the art for novel and efficient preparation methods for UDP-rhamnose.

### SUMMARY OF THE INVENTION

The present disclosure encompasses, in various embodiments, a biosynthetic method of preparing UDP-rhamnose. In a preferred embodiment, the present disclosure relates to a biosynthetic method of preparing uridine diphosphate beta-L-rhamnose (“UDP-L-rhamnose” or “UDP-L-R” or “UDP-L-Rh”). Generally, the method includes incubating uridine diphosphate-glucose (“UDP-glucose” or “UDPG”) with one or more recombinant polypeptides in the presence of NAD⁺ and a source of NADPH for a sufficient time to produce UDP-rhamnose, where the one or more recombinant polypeptides individually or collectively have UDP-rhamnose synthase activity.

In some embodiments, the one or more recombinant polypeptides can be a trifunctional enzyme having UDP-glucose 4,6-dehydratase, UDP-4-keto-6-deoxy-glucose 3,5-epimerase, and UDP-4-keto-rhamnose 4-keto-reductase activities. Such a trifunctional polypeptide is also referred as an RHM enzyme. In such embodiments, the one or more recombinant polypeptides can be selected from an RHM enzyme from Ricinus communis, Ceratopteris thalictroides, Azolla filiculoides, Ostreococcus lucimarinus, Nannochloropsis oceanica, Ulva lactuca, Golenkinia longispicula, Tetraselrnis subcordiformis or Tetraselrnis cordiformis. In these embodiments, the one or more recombinant polypeptides can be selected from a recombinant polypeptide comprising an amino acid sequence having at least 80%, at least 85%, at least 90%, at least 95%, at least 96%, at least 97%, at least 98%, at least 99%, or 100% sequence identity to SEQ ID NO: 1, SEQ ID NO: 3, SEQ ID NO: 5, SEQ ID NO: 39, SEQ ID NO: 41, SEQ ID NO: 43, SEQ ID NO: 45, SEQ ID NO: 47, or SEQ ID NO: 89. These one or more recombinant polypeptides can be selected from a recombinant polypeptide coded by a nucleotide comprising a nucleotide sequence having at least 80%, at least 85%, at least 90%, at least 95%, at least 96%, at least 97%, at least 98%, at least 99%, or 100% sequence identity to SEQ ID NO: 2, SEQ ID NO: 4, SEQ ID NO: 6, SEQ ID NO: 40, SEQ ID NO: 42, SEQ ID NO: 44, SEQ ID NO: 46, SEQ ID NO: 48, or SEQ ID NO: 90.

In certain embodiments, the one or more recombinant polypeptides can comprise a first recombinant polypeptide and a second recombinant polypeptide, where the first recombinant polypeptide and the second recombinant polypeptide collectively have UDP-rhamnose synthase activity. Specifically, the first recombinant polypeptide can have primarily UDP-glucose 4,6-dehydratase activity and such recombinant polypeptide is referred herein as a “DH” (dehydratase) enzyme. The second recombinant polypeptide can be a bifunctional recombinant polypeptide having both UDP-4-keto-6-deoxy-glucose 3,5-epimerase and UDP-4-keto-rhamnose 4-keto-reductase activities. This bifunctional recombinant polypeptide is referred herein as an “ER” enzyme (the letter “E” standing for epimerase activity and the letter “R” standing for reductase activity).

In such embodiments, the first recombinant polypeptide can be selected from a DH enzyme from Botrytis cinerea, Acrostichum aureum, Ettlia oleoabundans, Volvox carteri, Chlamydomonas reinhardtii, Oophila amblystomatis, or Dunaliella primolecta. In these embodiments, the first recombinant polypeptides can be selected from a recombinant polypeptide comprising an amino acid sequence having at least 80%, at least 85%, at least 90%, at least 95%, at least 96%, at least 97%, at least 98%, at least 99%, or 100% sequence identity to SEQ ID NO: 7, SEQ ID NO: 27, SEQ ID NO: 29, SEQ ID NO: 31, SEQ ID NO: 33, SEQ ID NO: 35, or SEQ ID NO: 37. Such first recombinant polypeptides can be selected from a recombinant polypeptide coded by a nucleotide comprising a nucleotide sequence having at least 80%, at least 85%, at least 90%, at least 95%, at least 96%, at least 97%, at least 98%, at least 99%, or 100% sequence identity to SEQ ID NO: 8, SEQ ID NO: 28, SEQ ID NO: 30, SEQ ID NO: 32, SEQ ID NO: 34, SEQ ID NO: 36, or SEQ ID NO: 38.

Examples of suitable second recombinant polypeptides can include an ER enzyme from Physcomitrella patens subsp. Patens, Pyricularia oryzae, Nannochloropsis oceanica, Ulva lactuca, Tetraselrnis cordiformis, Tetraselrnis subcordiformis, Chlorella sorokiniana, Chlamydomonas moewusii, Golenkinia longispicula, Chlamydomonas reinhardtii, Chromochloris zofingiensis, Dunaliella primolecta, Pavlova lutheri, Nitella mirabilis, Marchantia polymorpha, Selaginella moellendorffii, Bryum argenteum var argenteum, Arabidopsis thaliana, Pyricularia oryzae, or Citrus clementina. For example, the second recombinant polypeptide can be selected from a recombinant polypeptide comprising an amino acid sequence having at least 80%, at least 85%, at least 90%, at least 95%, at least 96%, at least 97%, at least 98%, at least 99%, or 100% sequence identity to SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 91, SEQ ID NO: 93, or SEQ ID NO: 95. Such second recombinant polypeptides can be selected from a recombinant polypeptide coded by a nucleotide comprising a nucleotide sequence having at least 80%, at least 85%, at least 90%, at least 95%, at least 96%, at least 97%, at least 98%, at least 99%, or 100% sequence identity to SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 92, SEQ ID NO: 94, or SEQ ID NO: 96.

In yet other embodiments, the one or more recombinant polypeptides can be a fusion enzyme comprising a first domain having UDP-glucose 4,6-dehydratase activity (a DH domain) and a second domain having bifunctional ER activity (that is, both UDP-4-keto-6-deoxy-glucose 3,5-epimerase and UDP-4-keto-rhamnose 4-keto-reductase activities). The DH domain can be coupled to the ER domain via a peptide linker. In various embodiments, the peptide linker can comprise 2-15 amino acids. Exemplary linkers include those comprising glycine and serine, for example, repeat units of glycine, repeat units of serine, repeat units of certain motifs consisting of glycine and serine, and combinations thereof. In preferred embodiments, the peptide linker can be GSG. Such a fusion enzyme therefore includes a DH domain fused to an ER domain which collectively have UDP-rhamnose synthase activity and have the capacity to catalyze the conversion of UDP-glucose to UDP-rhamnose.

In embodiments involving fusion enzymes, the first domain of the fusion enzyme can comprise a DH enzyme from Botrytis cinerea, Acrostichum aureum, Ettlia oleoabundans, Volvox carteri, Chlamydomonas reinhardtii, Oophila amblystomatis, or Dunaliella primolecta. In these embodiments, the first domain can comprise a recombinant polypeptide comprising an amino acid sequence having at least 80%, at least 85%, at least 90%, at least 95%, at least 96%, at least 97%, at least 98%, at least 99%, or 100% sequence identity to SEQ ID NO: 7, SEQ ID NO: 27, SEQ ID NO: 29, SEQ ID NO: 31, SEQ ID NO: 33, SEQ ID NO: 35, or SEQ ID NO: 37. Such DH domain can comprise a recombinant polypeptide coded by a nucleotide comprising a nucleotide sequence having at least 80%, at least 85%, at least 90%, at least 95%, at least 96%, at least 97%, at least 98%, at least 99%, or 100% sequence identity to SEQ ID NO: 8, SEQ ID NO: 28, SEQ ID NO: 30, SEQ ID NO: 32, SEQ ID NO: 34, SEQ ID NO: 36, or SEQ ID NO: 38. The second domain of the fusion enzyme can comprise an ER enzyme from Physcomitrella patens subsp. Patens, Pyricularia oryzae, Nannochloropsis oceanica, Ulva lactuca, Tetraselrnis cordiformis, Tetraselrnis subcordiformis, Chlorella sorokiniana, Chlamydomonas moewusii, Golenkinia longispicula, Chlamydomonas reinhardtii, Chromochloris zofingiensis, Dunaliella primolecta, Pavlova lutheri, Nitella mirabilis, Marchantia polymorpha, Selaginella moellendorffii, Bryum argenteum var argenteum, Arabidopsis thaliana, Pyricularia oryzae, or Citrus clementina. For example, the ER domain can comprise a recombinant polypeptide comprising an amino acid sequence having at least 80%, at least 85%, at least 90%, at least 95%, at least 96%, at least 97%, at least 98%, at least 99%, or 100% sequence identity to SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 91, SEQ ID NO: 93, or SEQ ID NO: 95. Such ER domain can comprise a recombinant polypeptide coded by a nucleotide comprising a nucleotide sequence having at least 80%, at least 85%, at least 90%, at least 95%, at least 96%, at least 97%, at least 98%, at least 99%, or 100% sequence identity to SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 92, SEQ ID NO: 94, or SEQ ID NO: 96. In certain preferred embodiments, the first domain of the fusion enzyme can comprise an amino acid sequence having at least 80% sequence identity to SEQ ID NO: 7. The second domain can comprise an amino acid sequence having at least 80% sequence identity to SEQ ID NO: 91, SEQ ID NO: 93, SEQ ID NO: 95, SEQ ID NO: 61, or SEQ ID NO: 63. In such preferred embodiments, the fusion enzyme as a whole can comprise an amino acid sequence having at least 80%, at least 85%, at least 90%, at least 95%, at least 96%, at least 97%, at least 98%, at least 99%, or 100% sequence identity to SEQ ID NO: 9, SEQ ID NO: 11, or SEQ ID NO: 13, SEQ ID NO: 83, or SEQ ID NO: 85. In certain preferred embodiments, the first domain of the fusion enzyme can comprise an amino acid sequence having at least 80% sequence identity to SEQ ID NO: 7 or SEQ ID NO: 31, and the second domain of the fusion enzyme can comprise an amino acid sequence having at least 80% sequence identity to SEQ ID NO: 63. The fusion enzyme as a whole can comprise an amino acid sequence having at least 80%, %, at least 85%, at least 90%, at least 95%, at least 96%, at least 97%, at least 98%, at least 99%, or 100% sequence identity to SEQ ID NO: 87.

In some embodiments, the first recombinant polypeptide can include an amino acid sequence having at least 80%, at least 85%, at least 90%, at least 95%, at least 96%, at least 97%, at least 98%, at least 99%, or 100% sequence identity to SEQ ID NO: 1, SEQ ID NO: 3, SEQ ID NO: 5, SEQ ID NO: 9, SEQ ID NO: 11, SEQ ID NO: 13, SEQ ID NO. 39, SEQ ID NO. 41, SEQ ID NO. 43, SEQ ID NO. 45, SEQ ID NO. 47, SEQ ID NO. 83, SEQ ID NO. 85, SEQ ID NO. 87 or SEQ ID NO. 89. In some embodiments, the first recombinant polypeptide can include an amino acid sequence having at least 90% sequence identity to SEQ ID NO: 9. In some embodiments, the first recombinant polypeptide can include an amino acid sequence having at least 90% sequence identity to SEQ ID NO: 11. In some embodiments, the first recombinant polypeptide can include an amino acid sequence having at least 90% sequence identity to SEQ ID NO: 13. In some embodiments, the first recombinant polypeptide can include an amino acid sequence having at least 90% sequence identity to SEQ ID NO: 83. In some embodiments, the first recombinant polypeptide can include an amino acid sequence having at least 90% sequence identity to SEQ ID NO: 85. In some embodiments, the first recombinant polypeptide can include an amino acid sequence having at least 90% sequence identity to SEQ ID NO: 87. In some embodiments, the first recombinant polypeptide can include an amino acid sequence having at least 90% sequence identity to SEQ ID NO: 89.

In various embodiments, biosynthetic methods provided herein can include expressing the first recombinant polypeptide in a transformed cellular system. In some embodiments, the transformed cellular system is selected from the group consisting of a yeast, a non-UDP-rhamnose producing plant, an alga, a fungus, and a bacterium. In some embodiments, the bacterium or yeast can be selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium.

In some embodiments, the source of NADPH can be provided after incubating the uridine diphosphate-glucose with the first recombinant polypeptide for a sufficient time to generate UDP-4-keto-6-deoxy-glucose (“UDP4K6G”). In some embodiments, the source of NADPH can include an oxidation reaction substrate and an NADP⁺-dependent enzyme. In some embodiments, the source of NADPH can include malate and a malic enzyme. In some embodiments, the source of NADPH can include formate and formate dehydrogenase. In some embodiments, the source of NADPH can include phosphite and phosphite dehydrogenase.

In some embodiments, the incubating step can be performed in the transformed cellular system. In other embodiments, the incubating step can be performed in vitro. In some embodiments, biosynthetic methods disclosed herein can include isolating the first recombinant polypeptide from the transformed cellular system and performing the incubating step in vitro.

In some embodiments, the first recombinant polypeptide having rhamnose synthase activity and a second recombinant polypeptide having sucrose synthase activity are incubated in a medium comprising sucrose and uridine diphosphate (“UDP”). The second recombinant polypeptide can be selected from the group consisting of an Arabidopsis sucrose synthase, a Vigna radiate sucrose synthase, and a Coffea sucrose synthase. In this embodiment, in the first step of the reaction, sucrose synthase activity yields UDP-glucose which in turn is used as a substrate by the first recombinant enzyme to yield UDP-rhamnose. The source of NADPH in this embodiment can include an oxidation reaction substrate and an NADP⁺-dependent enzyme. In some embodiments, the source of NADPH can include malate and a malic enzyme. In some embodiments, the source of NADPH can include formate and formate dehydrogenase. In some embodiments, the source of NADPH can include phosphite and phosphite dehydrogenase.

Also provided herein, inter alia, are biosynthetic methods of preparing a steviol glycoside composition comprising at least one rhamnose-containing steviol glycoside. The methods can include incubating UDP-glucose with a first recombinant polypeptide having UDP-rhamnose synthase activity, in the presence of NAD⁺ and a source of NADPH, to produce UDP-rhamnose; and reacting the UDP-rhamnose with a steviol glycoside substrate in the presence of a second recombinant polypeptide having UDP-rhamnosyltransferase activity, so that a rhamnose moiety is coupled to the steviol glycoside substrate to produce at least one rhamnose-containing steviol glycoside. In some embodiments, the steviol glycoside substrate can be Reb A and the resulting steviol glycoside composition can include Reb N, Reb J, or both.

Aspects of the present disclosure also provide a steviol glycoside composition that includes at least one rhamnose-containing steviol glycoside obtainable by or produced by any biosynthetic method described herein, including any of the above-mentioned embodiments.

Aspects of the present disclosure also provide a nucleic acid encoding a polypeptide as described herein. In some embodiments, the nucleic acid comprises a sequence encoding a polypeptide comprising an amino acid sequence having at least 80%, at least 85%, at least 90%, at least 95%, at least 96%, at least 97%, at least 98%, at least 99%, or 100% identity to SEQ ID NO: 1, SEQ ID NO: 3, SEQ ID NO: 5, SEQ ID NO: 9, SEQ ID NO: 11, SEQ ID NO: 13, SEQ ID NO. 39, SEQ ID NO. 41, SEQ ID NO. 43, SEQ ID NO. 45, SEQ ID NO. 47, SEQ ID NO. 83, SEQ ID NO. 85, SEQ ID NO. 87 or SEQ ID NO. 89. In some embodiments, the nucleic acid comprises the sequence of SEQ ID NO: 2. In some embodiments, the nucleic acid comprises the sequence of SEQ ID NO: 4. In some embodiments, the nucleic acid comprises the sequence of SEQ ID NO: 6. In some embodiments, the nucleic acid comprises the sequence of SEQ ID NO: 10. In some embodiments, the nucleic acid comprises the sequence of SEQ ID NO: 12. In some embodiments, the nucleic acid comprises the sequence of SEQ ID NO: 14. In some embodiments, the nucleic acid comprises the sequence of SEQ ID NO: 40. In some embodiments, the nucleic acid comprises the sequence of SEQ ID NO: 42. In some embodiments, the nucleic acid comprises the sequence of SEQ ID NO: 44. In some embodiments, the nucleic acid comprises the sequence of SEQ ID NO: 46. In some embodiments, the nucleic acid comprises the sequence of SEQ ID NO: 84. In some embodiments, the nucleic acid comprises the sequence of SEQ ID NO: 86. In some embodiments, the nucleic acid comprises the sequence of SEQ ID NO: 88. In some embodiments, the nucleic acid comprises the sequence of SEQ ID NO: 90. In some embodiments, the nucleic acid is a plasmid or other vector.

Aspects of the present disclosure also provide a cell comprising a nucleic acid described herein, including any of the above-mentioned embodiments.

Aspects of the present disclosure provide a cell comprising at least one polypeptide comprising an amino acid sequence having at least 80%, at least 85%, at least 90%, at least 95%, at least 96%, at least 97%, at least 98%, at least 99%, or 100% identity to SEQ ID NO: 1, SEQ ID NO: 3, SEQ ID NO: 5, SEQ ID NO: 9, SEQ ID NO: 11, SEQ ID NO: 13, SEQ ID NO. 39, SEQ ID NO. 41, SEQ ID NO. 43, SEQ ID NO. 45, SEQ ID NO. 47, SEQ ID NO. 83, SEQ ID NO. 85, SEQ ID NO. 87 or SEQ ID NO. 89. In some embodiments, the cell comprises at least one polypeptide comprising the sequence of SEQ ID NO: 1. In some embodiments, the cell comprises at least one polypeptide comprising the sequence of SEQ ID NO: 3. In some embodiments, the cell comprises at least one polypeptide comprising the sequence of SEQ ID NO: 9. In some embodiments, the cell comprises at least one polypeptide comprising the sequence of SEQ ID NO: 9. In some embodiments, the cell comprises at least one polypeptide comprising the sequence of SEQ ID NO: 11. In some embodiments, the cell comprises at least one polypeptide comprising the sequence of SEQ ID NO: 13. In some embodiments, the cell comprises at least one polypeptide comprising the sequence of SEQ ID NO: 37. In some embodiments, the cell comprises at least one polypeptide comprising the sequence of SEQ ID NO: 41. In some embodiments, the cell comprises at least one polypeptide comprising the sequence of SEQ ID NO: 43. In some embodiments, the cell comprises at least one polypeptide comprising the sequence of SEQ ID NO: 45. In some embodiments, the cell comprises at least one polypeptide comprising the sequence of SEQ ID NO: 47. In some embodiments, the cell comprises at least one polypeptide comprising the sequence of SEQ ID NO: 83. In some embodiments, the cell comprises at least one polypeptide comprising the sequence of SEQ ID NO: 85. In some embodiments, the cell comprises at least one polypeptide comprising the sequence of SEQ ID NO: 87. In some embodiments, the cell comprises at least one polypeptide comprising the sequence of SEQ ID NO: 89. In some embodiments, the cell is a yeast cell, a non-UDP-rhamnose producing plant cell, an algal cell, a fungal cell, or a bacterial cell. In some embodiments, the bacterium or yeast cell is selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; and Clostridium. In some embodiments, the cell further comprises one or more other polypeptides having UDP-rhamnosyltransferase activity, UDP-glucosyltransferase activity, and/or sucrose synthase activity as described herein.

As for the cellular system in the embodiment, it can be selected from the group consisting of one or more bacteria, one or more yeasts, and a combination thereof, or any cellular system that would allow the genetic transformation with the selected genes and thereafter the biosynthetic production of UDP-rhamnose. In a most preferred microbial system, E. coli is used to produce the desired compound.

Other aspects of the present disclosure provide an in vitro reaction mixture comprising at least one polypeptide comprising an amino acid sequence having at least 80%, at least 85%, at least 90%, at least 95%, at least 96%, at least 97%, at least 98%, at least 99%, or 100% identity to SEQ ID NO: 1, SEQ ID NO: 3, SEQ ID NO: 5, SEQ ID NO: 9, SEQ ID NO: 11, SEQ ID NO: 13, SEQ ID NO. 39, SEQ ID NO. 41, SEQ ID NO. 43, SEQ ID NO. 45, SEQ ID NO. 47, SEQ ID NO. 83, SEQ ID NO. 85, SEQ ID NO. 87 or SEQ ID NO. 89. In some embodiments, the in vitro reaction mixture comprises at least one polypeptide comprising the sequence of SEQ ID NO: 1. In some embodiments, the in vitro reaction mixture comprises at least one polypeptide comprising the sequence of SEQ ID NO: 3. In some embodiments, the in vitro reaction mixture comprises at least one polypeptide comprising the sequence of SEQ ID NO: 5. In some embodiments, the in vitro reaction mixture comprises at least one polypeptide comprising the sequence of SEQ ID NO: 9. In some embodiments, the in vitro reaction mixture comprises at least one polypeptide comprising the sequence of SEQ ID NO: 11. In some embodiments, the in vitro reaction mixture comprises at least one polypeptide comprising the sequence of SEQ ID NO: 13. In some embodiments, the in vitro reaction mixture comprises at least one polypeptide comprising the sequence of SEQ ID NO: 37. In some embodiments, the in vitro reaction mixture comprises at least one polypeptide comprising the sequence of SEQ ID NO: 41. In some embodiments, the in vitro reaction mixture comprises at least one polypeptide comprising the sequence of SEQ ID NO: 43. In some embodiments, the in vitro reaction mixture comprises at least one polypeptide comprising the sequence of SEQ ID NO: 45. In some embodiments, the in vitro reaction mixture comprises at least one polypeptide comprising the sequence of SEQ ID NO: 47. In some embodiments, the in vitro reaction mixture comprises at least one polypeptide comprising the sequence of SEQ ID NO: 83. In some embodiments, the in vitro reaction mixture comprises at least one polypeptide comprising the sequence of SEQ ID NO: 85. In some embodiments, the in vitro reaction mixture comprises at least one polypeptide comprising the sequence of SEQ ID NO: 87. In some embodiments, the in vitro reaction mixture comprises at least one polypeptide comprising the sequence of SEQ ID NO: 89. In some embodiments, the in vitro reaction mixture further comprises one or more other recombinant polypeptides having UDP-rhamnosyltransferase activity, UDP-glucosyltransferase activity, and/or sucrose synthase activity as described herein.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

FIG. 1 shows the chemical structure of uridine diphosphate beta-L-rhamnose.

FIG. 2 is a schematic diagram illustrating a multi-enzyme synthetic pathway for (a) producing UDP-rhamnose from UDP-glucose; (b) producing Reb N from Reb A and UDP-rhamnose via the intermediate Reb J; (c) regenerating NADPH from NADP⁺ and malate using malic enzyme MaeB; and (d) regenerating UDP-glucose (UDPG) from UDP and sucrose using sucrose synthase according to the present disclosure.

FIG. 3 shows the UDP-rhamnose biosynthetic pathway in plants and fungi involving three different enzymes. In the first step of this biosynthetic pathway, UDP-glucose 4,6 dehydratase converts UDP-glucose into UDP-4-keto-6-deoxy glucose (“UDP4K6G”). In the second step of this biosynthetic pathway, the enzyme UDP-4-keto-6-deoxy-glucose 3,5 epimerase converts UDP-4-keto-6-deoxy glucose into UDP-4-keto rhamnose. At the third enzymatic step in this biosynthetic pathway, UDP-4-keto rhamnose-4-ketoreductase convert UDP-4-keto rhamnose into UDP-rhamnose. Trifunctional polypeptides having the activity of all three enzymes are referred as “RHM” enzymes. Bifunctional polypeptides having UDP-4-keto-6-deoxy-glucose 3,5 epimerase and UDP-4-keto rhamnose-4-ketoreductase activities are referred as “ER” enzymes. Polypeptides having only UDP-glucose 4,6 dehydratase activity are referred as “DH” enzymes. In addition, in this embodiment, the NADPH cofactor is regenerated by the oxidation of malate into pyruvate using NADP⁺ as the oxidizing agent, and the reaction is catalyzed by an NADP⁺-dependent malic enzyme (MaeB). In addition, in the embodiment, UDP-glucose can be converted from UDP and sucrose by sucrose synthase (SUS).

FIG. 4 shows a one-pot multi-enzyme system for the in vitro synthesis of UDP-rhamnose using a trifunctional UDP-rhamnose synthase (e.g., NRF1 or NR32) for the bioconversion of UDP-glucose (UDPG) to UDP-rhamnose according to the present disclosure. UDP-glucose can be replenished from UDP and sucrose in a reaction catalyzed by a sucrose synthase (SUS) as shown. The synthesis of UDP-rhamnose can be coupled with an oxidation reaction to regenerate the NADPH cofactor. In the embodiment shown, the NADPH cofactor is regenerated by the oxidation of formate into carbon dioxide using NADP⁺ as the oxidizing agent, and the reaction is catalyzed by a formate dehydrogenase (FDH).

FIG. 5 shows a one-pot multi-enzyme system for the in vitro synthesis of UDP-rhamnose using a trifunctional UDP-rhamnose synthase (e.g., NRF1 or NR32) for the bioconversion of UDP-glucose to UDP-rhamnose according to the present disclosure. UDP-glucose can be replenished from UDP and sucrose in a reaction catalyzed by sucrose synthase (SUS) as shown. The synthesis of UDP-rhamnose can be coupled with an oxidation reaction to regenerate the NADPH cofactor. In the embodiment shown, the NADPH cofactor is regenerated by the oxidation of phosphite into phosphate using NADP⁺ as the oxidizing agent, and the reaction is catalyzed by a phosphite dehydrogenase (PTDH).

FIG. 6 shows the results of enzymatic activity analyses of three trifunctional UDP-rhamnose synthase candidates (NR12, NR32 and NR33) for UDP-rhamnose production. The letter “a” next to an enzyme refers to a one-step cofactor addition approach under which both NAD⁺ and NADPH were added at the beginning of the reaction. The letter “b” next to an enzyme refers to a two-step cofactor addition approach under which NAD⁺ was added at the beginning of the reaction but NADPH was not added until 3 hours into the reaction. All samples were collected after 3 hours (A), after 6 hours (B), and after 18 hours (C). Collected samples were extracted by chloroform and analyzed by HPLC. Legend: “UDP-Rh”=UDP-rhamnose; “UDPG”=UDP-glucose; and “UDP4K6G”=UDP-4-keto-6-deoxyglucose.

FIG. 7 shows how the two-step cofactor addition approach according to the present disclosure can enhance the conversion efficiency for UDP-rhamnose production. In this experiment, the recombinant UDP-rhamnose synthase enzyme NRF1 was used. Collected samples were extracted by chloroform and analyzed by HPLC. All samples were collected after 1 hr, 3 hr, 4 hr, 6 hr and 18 hr. The letter “a” next to a reaction time refers to a one-step cofactor addition approach under which both NAD⁺ and NADPH were added at the beginning of the reaction. The letter “b” next to a reaction time refers to a two-step cofactor addition approach under which NAD⁺ was added at the beginning of the reaction but NADPH was not added until 3 hours into the reaction. Legend: “UDP-Rh”=UDP-rhamnose; “UDPG”=UDP-glucose; and “UDP4K6G”=UDP-4-keto-6-deoxyglucose.

FIG. 8 compares the production of UDP-glucose (UDPG), UDP-4-keto-6-deoxyglucose (UDP4K6G), and UDP-rhamnose (UDP-Rh) using different one-pot multi-enzyme reaction systems. FIG. 8, panel A shows the results after 6 hours of reaction time. FIG. 8, panel B shows the results after 18 hours of reaction time. Details of the reaction systems 1-6 are summarized in Table 2.

FIG. 9. Enzymatic analysis of DH candidates for UDP-4-keto-6-deoxy-glucose (UDP4K6G) production. The DH candidates included in this experiment were NR55N, NR60N, NR66N, NR67N, NR68N and NR69N. Also included in this experiment were the following RHM candidates having trifunctional enzyme activities: NR53N, NR58N, NR62N, NR64N and NR65N. All samples were collected at 18 hr. Collected samples were extracted by chloroform and analyzed by HPLC. “UDP-Rh”: UDP-rhamnose; “UDPG”: UDP-glucose; “UDP4K6G”: UDP-4-keto-6-deoxy-glucose. “Control”: Reaction without enzyme addition.

FIG. 10. Enzymatic analysis of ER candidates for bioconversion of UDP-4-keto-6-deoxy-glucose (UDP4K6G) to UDP-β-L-rhamnose. All samples were collected at 18 hr. Collected samples were extracted by chloroform and analyzed by HPLC. “UDP-Rh”: UDP-rhamnose; “UDPG”: UDP-glucose; “UDP4K6G”: UDP-4-keto-6-deoxy-glucose.

FIG. 11. Comparison of the enzymatic activity of three fusion enzymes (NRF3, NRF2, and NRF1) against a DH enzyme (NX10) for UDP-rhamnose production. NAD⁺ was added at the beginning of the reaction and NADPH was added 3 hours after the reaction has begun. All samples were collected at 21 hours. Collected samples were extracted by chloroform and analyzed by HPLC. Legend: “UDP-Rh”=UDP-rhamnose; “UDPG”=UDP-glucose; and “UDP4K6G”=UDP-4-keto-6-deoxyglucose.

FIG. 12. Enzymatic analysis of fusion enzymes for UDP-rhamnose production. NAD⁺ was added in the initial reaction and NADPH was added in the reaction after 3 hr. All samples were collected at 21 hr. Collected samples were extracted by chloroform and analyzed by HPLC. “UDP-Rh”: UDP-rhamnose; “UDPG”: UDP-glucose; “UDP4K6G”: UDP-4-keto-6-deoxyglucose.

FIG. 13 shows the production of UDP-4-keto-6-deoxy glucose (UDP4K6G) and UDP-rhamnose (UDP-Rh) using a one-pot multi-enzyme reaction system optimized for the in vitro synthesis of UDP-rhamnose. In this embodiment, NRF1 was used as the RHM enzyme. The two-step cofactor addition approach was used, with NAD⁺ being added at the beginning of the reaction, and NADP⁺, MaeB, and malate were added after 3 hours to regenerate NADPH. The products were analyzed after 3 hours and after 18 hours of reaction time.

FIG. 14 shows HPLC spectra confirming the in vitro production of Reb J and Reb N from Reb A as catalyzed by selected UDP-rhamnosyltransferase (1,2 RhaT) and UDP-glucosyltransferase (UGT) according to the present disclosure. FIG. 14, panel A shows the Reb J standard. FIG. 14, panel B shows the Reb N standard. FIG. 14, panel C shows that Reb J was enzymatically produced by EUCP1 as an exemplary 1,2 RhaT when the product was measured at 22-hr. FIG. 14, panel D shows that Reb N was enzymatically produced from the Reb J product by CP1 as an exemplary UGT when the product was measured at 25-hr.

### DETAILED DESCRIPTION

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

Cellular system is any cells that provide for the expression of ectopic proteins. It included bacteria, yeast, plant cells and animal cells. It includes both prokaryotic and eukaryotic cells. It also includes the in vitro expression of proteins based on cellular components, such as ribosomes.

Coding sequence is to be given its ordinary and customary meaning to a person of ordinary skill in the art and is used without limitation to refer to a DNA sequence that encodes for a specific amino acid sequence.

The term “growing the cellular system” means providing an appropriate medium that would allow cells to multiply and divide. It also includes providing resources so that cells or cellular components can translate and make recombinant proteins.

Protein expression can occur after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

According to the current disclosure, a yeast as claimed herein are eukaryotic, single-celled microorganisms classified as members of the fungus kingdom. Yeasts are unicellular organisms which evolved from multicellular ancestors but with some species useful for the current disclosure being those that have the ability to develop multicellular characteristics by forming strings of connected budding cells known as pseudo hyphae or false hyphae.

The names of the UGT enzymes used in the present disclosure are consistent with the nomenclature system adopted by the UGT Nomenclature Committee (Mackenzie et al., “The UDP glycosyltransferase gene super family: recommended nomenclature updated based on evolutionary divergence,” PHARMACOGENETICS, 1997, vol. 7, pp. 255-269), which classifies the UGT genes by the combination of a family number, a letter denoting a subfamily, and a number for an individual gene. For example, the name “UGT76G1” refers to a UGT enzyme encoded by a gene belonging to UGT family number 76 (which is of plant origin), subfamily G, and gene number 1.

The term “complementary” is to be given its ordinary and customary meaning to a person of ordinary skill in the art and is used without limitation to describe the relationship between nucleotide bases that are capable of hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing one or more chemical or biological entities which are distinctly different from the initial starting entities.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxy inosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably and are used without limitation to refer to a polymer of amino acids, or amino acid analogs, regardless of its size or function. Although the term “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a common evolutionary origin, including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it can affect the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the craft and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

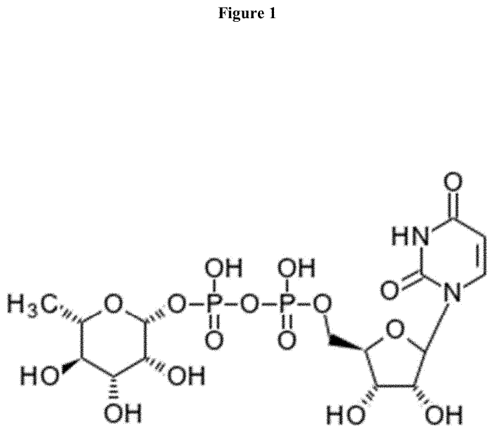

The present disclosure relates, in some embodiments, to the biosynthetic production of UDP-rhamnose. In a preferred embodiment, the present invention relates to the production of UDP-L-rhamnose, the chemical structure of which is shown in FIG. 1. Because UDP-rhamnose can be used as a rhamnose donor moiety in the biosynthetic production of rhamnose-containing steviol glycosides such as Reb J and Reb N, the present disclosure also relates, in part, to biosynthetic pathways for preparing rhamnose-containing steviol glycosides that include the preparation of UDP-rhamnose, for example, from UDP-glucose.

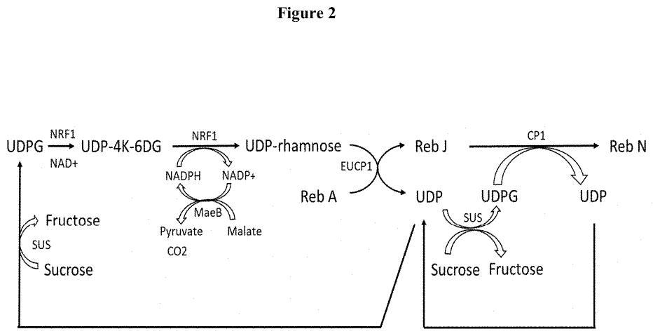

Referring to FIG. 2, aspects of the present disclosure relate to a reaction system that includes, at a minimum, a first recombinant polypeptide having UPD-rhamnose synthase activity that catalyzes the bioconversion of UDP-rhamnose from UDP-glucose via the intermediate UDP-4-keto-6-deoxyglucose (“UDP4K6G”). In the embodiments illustrated in FIG. 2, the first recombinant polypeptide is a trifunctional enzyme that catalyzes both the bioconversion of UDP-glucose to UDP4K6G, and the bioconversion of UDP4K6G to UDP-rhamnose. In some embodiments, the first polypeptide can include two different enzymes each responsible for a different step in the bioconversion. The reaction system also can include a second polypeptide that catalyzes a reaction for the regeneration of NADPH, which is a cofactor used in the bioconversion of UDP-glucose to UDP-rhamnose. The reaction system can further include a third recombinant polypeptide that converts UDP and sucrose into UDP-glucose. In embodiments where the UDP-rhamnose is used as a rhamnose donor moiety in the biosynthetic production of rhamnose-containing steviol glycosides such as Reb J and Reb N, the reaction system can include additional enzymes having rhamnosyltransferase and glycosyltransferase activities.

UDP-rhamnose biosynthetic pathway in plants and fungi involves three different enzymes. In the first step of this biosynthetic pathway, UDP-glucose 4,6 dehydratase (“DH”) converts UDP-glucose into UDP-4-keto-6-deoxy glucose (UDP4K6G). In the second step of this biosynthetic pathway, the enzyme UDP-4-keto-6-deoxy-glucose 3,5 epimerase converts UDP-4-keto-6-deoxy glucose into UDP-4-keto rhamnose. At the third enzymatic step in this biosynthetic pathway, UDP-4-keto rhamnose-4-ketoreductase convert UDP-4-keto rhamnose in to UDP-rhamnose. In various embodiments, the present invention provides trifunctional recombinant polypeptides having UDP-glucose 4,6-dehydratase, UDP-4-keto-6-deoxy-glucose 3,5-epimerase, and UDP-4-keto-rhamnose 4-keto-reductase activities. Such a trifunctional polypeptide is also referred as RHM enzyme. Since the trifunctional recombinant polypeptides exhibit three different enzyme functions, this trifunctional recombinant protein is also referred as multi-enzyme protein.

In certain embodiments, the present invention provides recombinant polypeptide having only the activity of the UDP-glucose 4,6-dehydratase enzyme and that recombinant polypeptide is referred herein as the “DH” (dehydratase) polypeptide. In another embodiment, the present invention provides bifunctional recombinant polypeptide having both UDP-4-keto-6-deoxy-glucose 3,5-epimerase and UDP-4-keto-rhamnose 4-keto-reductase activities. This bifunctional recombinant polypeptide is referred herein as the “ER” (the letter “E” standing for epimerase activity and the letter “R” standing for reductase activity). In yet another embodiment, the present invention provides a recombinant fusion polypeptide wherein an enzyme having UDP-glucose 4,6-dehydratase activity (the DH polypeptide) is fused with a bifunctional ER polypeptide having both UDP-4-keto-6-deoxy-glucose 3,5-epimerase and UDP-4-keto-rhamnose 4-keto-reductase activities. Such a fusion polypeptide is found to have the capacity to catalyze the conversion of UDP-glucose to UDP-rhamnose.

The cofactor NAD⁺ is needed in the DH-catalyzed step and the cofactor NADPH is needed in the second of the ER-catalyzed step.

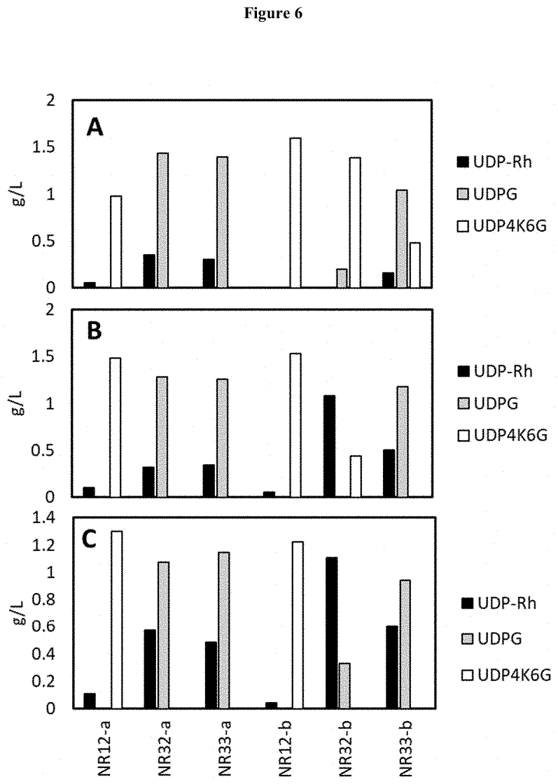

Referring to Table 1, the inventors have identified various trifunctional UDP-rhamnose synthase for the bioconversion of UDP-glucose to UDP-rhamnose. As shown in FIG. 6 below, NR12 from Ricinus communis [SEQ ID NO: 1], NR32 from Ceratopteris thalictroides [SEQ ID NO: 3] and NR33 from Azolla filiculoides [SEQ ID NO: 5] were shown as capable of catalyzing the conversion of UDP-glucose into UDP-rhamnose. Accordingly, in some embodiments, the present disclosure relates to a biosynthetic method for preparing UDP-rhamnose by incubating a recombinant polypeptide comprising an amino acid sequence having at least 80% sequence identity to SEQ ID NO: 1, SEQ ID NO: 3, or SEQ ID NO: 5, together with a substrate such as UDP-glucose, in the presence of cofactors NAD⁺ and NADPH.

In some embodiments, the present disclosure relates to a biosynthetic method for preparing UDP-rhamnose by incubating a substrate such as UDP-glucose with an artificial fusion enzyme obtained from the fusion of a high activity DH enzyme and a high activity ER enzyme. DH and ER enzymes can be obtained from a variety of sources as shown in the Examples below and their activities can be determined using biochemical assays. The nucleic acid sequence coding for a selected DH enzyme can be fused with the nucleic acid coding for a selected ER enzyme using the recombinant technologies well-known to a person skilled in the art to generate a recombinant fusion peptide catalyzing the synthesis of UDP-rhamnose from UDP-glucose. The DH enzyme and the ER enzyme can be coupled via a peptide linker. In various embodiments, the peptide linker can comprise 2-15 amino acids. Exemplary linkers include those comprising glycine and serine. In preferred embodiments, the DH enzyme and the ER enzyme can be coupled via a GSG linker (Table 3).

In various embodiments, UDP-glucose can be prepared in situ from UDP and sucrose in the presence of a sucrose synthase (SUS). For example, the SUS can have an amino acid sequence having at least 80% sequence identity to SEQ ID NO: 15.

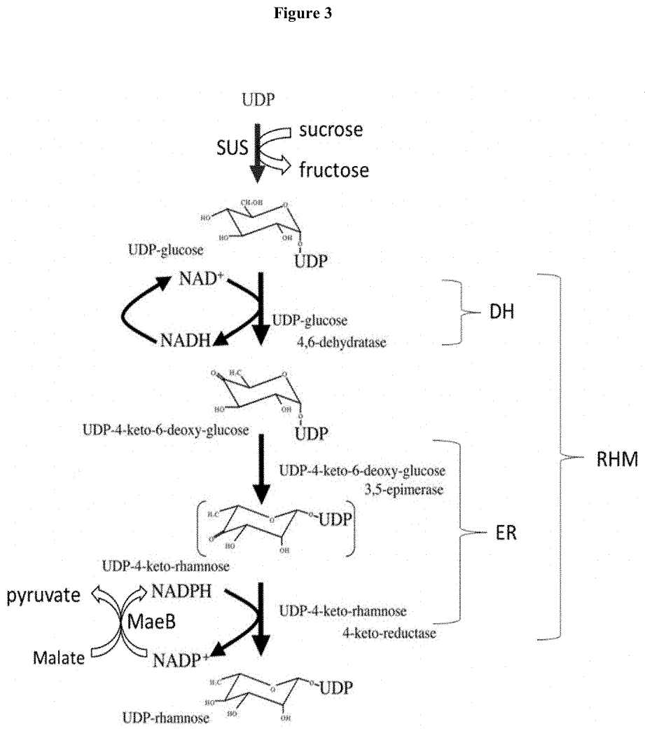

As shown in FIGS. 3-5, the present reaction system can include an NADP⁺-dependent enzyme and an oxidation reaction substrate for the regeneration of the cofactor NADPH. Referring to Figure. 3, the cofactor NAD⁺ is required in the DH-catalyzed reaction where UDP-glucose is converted to UDP-4-keto-6-deoxy-glucose. The UDP-4-keto-6-deoxy-glucose is then converted to UDP-4-keto-rhamnose by UDP-4-keto-6-deoxy-glucose 3,5-epimerase. The final step of catalytically converting UDP-4-keto-rhamnose into UDP-rhamnose by UDP-4-keto-rhamnose 4-keto-reductase requires the cofactor NADPH. Therefore, it is beneficial to incorporate a side reaction that can help regenerate the NADPH cofactor to ensure the continuous conversion of UDP-rhamnose.

With continued reference to FIG. 3, malate and an NADP⁺-dependent malic enzyme (“MaeB”) can be included to optimize the present pathway. As shown, malate is oxidized into pyruvate by MaeB in the presence of NADP⁺, over the course of which the NADP⁺ factor is reduced back into NADPH, hence regenerating NADPH for the bioconversion of UDP-rhamnose.

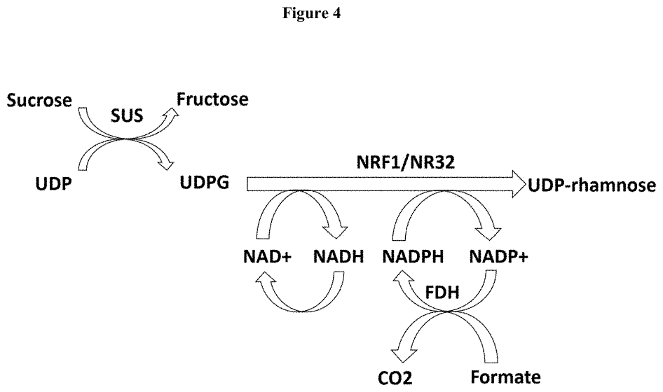

FIG. 4 shows an alternative embodiment where another NADP⁺-dependent enzyme, formate dehydrogenase (“FDH”), and formate are used. Similar to malate and MaeB, formate is oxidized into CO₂ by the FDH enzyme, which uses NADP⁺ as a cofactor. The electrons removed from formate are transferred to NADP⁺, which reduces NADP⁺ back into NADPH.

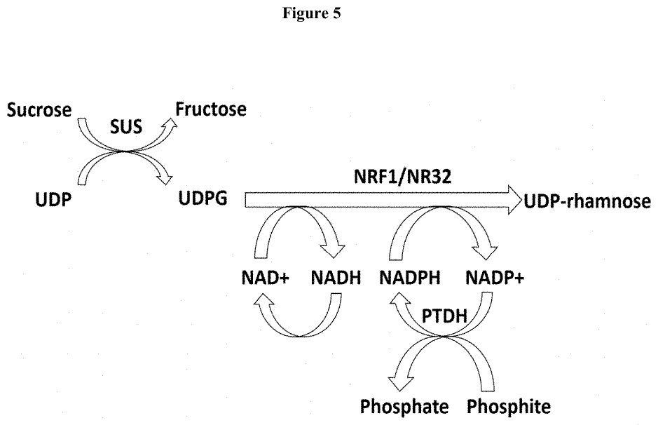

FIG. 5 shows yet another alternative embodiment for regenerating NADPH. Phosphite dehydrogenase (“PTDH”), another exemplary NADP⁺-dependent enzyme, is added with phosphite. Similar to malate and MaeB, phosphite is oxidized into phosphate by the PTDH enzyme, which uses NADP⁺ as a cofactor. The electrons removed from phosphite are transferred to NADP⁺, which reduces NADP⁺ back into NADPH.

Part of the present disclosure relates to the production of rhamnose-containing steviol glycosides using UDP-Rhamnose as the rhamnose donor moiety. Referring back to FIG. 2, a rhamnose-containing steviol glycoside such as Reb J and Reb N can be produced from Reb A. In some embodiments, Reb A can be converted to Reb J using a rhamnosyltransferase (RhaT) e.g., EU11 [SEQ ID No. 97], EUCP1 [SEQ ID No. 23], HV1 [SEQ ID No. 99], UGT2E-B [SEQ ID No. 101], or NX114 [SEQ ID No. 103], and a rhamnose donor moiety such as UDP-rhamnose. Subsequently, Reb J can be converted to Reb N using a UDP-glycosyltransferase (UGT) e.g., UGT76G1 [SEQ ID No. 107], CP1 [SEQ ID No. 25], CP2 [SEQ ID No. 105], or a fusion enzyme of UGT76G1 and SUS [SEQ ID No. 109].

### EXAMPLES

### Example 1

### Enzymatic Activity Screening of UDP-Rhamnose Synthase Enzymes

Phylogenetic, gene cluster, and protein BLAST analyses were used to identify candidate UDP-rhamnose synthase (“RHM”) genes for producing UDP-Rhamnose from UDP-glucose. Full-length DNA fragments of all candidate RHM genes were optimized and synthesized according to the codon preference of E. coli (Gene Universal, DE). The synthesized DNA fragments were cloned into a bacterial expression vector pETite N-His SUMO Kan Vector (Lucigen).

Each expression construct was transformed into E. coli BL21 (DE3), which was subsequently grown in LB media containing 50 μg/mL kanamycin at 37° C. until reaching an OD₆₀₀ of 0.8-1.0. Protein expression was induced by adding 1 mM of isopropyl β-D-1-thiogalactopyranoside (IPTG), and the culture was incubated further at 16° C. for 22 hours. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer, pH 7.2, 25 μg/ml lysozyme, 5 μg/ml DNase I, 20 mM imidazole, 500 mM NaCl, 10% glycerol, and 0.4% Triton X-100). The cells were disrupted by sonication at 4° C., and the cell debris was clarified by centrifugation (18,000×g; 30 min). The supernatant was loaded to an equilibrated (equilibration buffer: 50 mM potassium phosphate buffer, pH 7.2, 20 mM imidazole, 500 mM NaCl, 10% glycerol) Ni-NTA (Qiagen) affinity column. After loading of the protein samples, the column was washed with equilibration buffer to remove unbound contaminant proteins. The His-tagged RHM recombinant polypeptides were eluted with an equilibration buffer containing 250 mM of imidazole.

The purified candidate RHM recombinant polypeptides were assayed for UDP-rhamnose synthase activity by using UDP-glucose as substrate. Typically, the recombinant polypeptide (20-50 μg) was tested in a 200 μl in vitro reaction system. The reaction system contains 50 mM potassium phosphate buffer, pH 8.0, 3 mM MgCl₂, 3-6 mM UDP-glucose, 1-3 mM NAD⁺, 1 mM DTT and 1-3 mM NADPH. The reaction was performed at 30-37° C. and reaction was terminated by adding 200 μL chloroform. The samples were extracted with same volume chloroform by vertex for 10 mins. The supernatant was collected for high-performance liquid chromatography (HPLC) analysis after 10 mins centrifugation.

HPLC analysis was then performed using an Agilent 1200 system (Agilent Technologies, CA), including a quaternary pump, a temperature-controlled column compartment, an auto sampler and a UV absorbance detector. The chromatographic separation was performed using Dionex Carbo PA10 column (4×120 mm, Thermo Scientific) with mobile phase delivered at a flow rate of 1 ml/min. The mobile phase was H₂O (MPA) and 700 mM ammonium acetate (pH 5.2) (MPB). The gradient concentration of MPB was programmed for sample analysis. The detection wavelength used in the HPLC analysis was 261 nm. After activity screening, three RHM enzymes (NR12, NR32 and NR33) were identified as candidates for bioconversion of UDP-glucose to UDP-rhamnose (Table 1).

The activities of three different RHM enzymes namely NR12, NR32 and NR33 were studied for three different time period (3 hours, 6 hours and 18 hours). The enzyme activities at the end of three hours are shown in the top panel (A) of FIG. 6. The enzyme activities at the end of the six hours and 18 hours are shown in the middle panel (B) and bottom panel (C) of FIG. 6 respectively. In addition, in these experiments, an effort was also made to understand the effect of NADPH on the reduction of NAD⁺ during the action of UDP-glucose 4,6 dehydratase component of these three RHM enzymes. Under one experimental condition, the co-factors NAD⁺ and NADPH were added at the beginning of the experiment. This process variation is referred as “one-step cofactor addition” and is marked by the letter “a” after the enzyme name in FIG. 6 (NR12-a, NR32-a, and NR33-a). In the second set of experiments, NAD was added at the beginning of the experiment and NADPH was not added until 3 hours after the reaction had started. This process variation is referred as “two-step cofactor addition” which is marked by the letter “b” after the enzyme name in FIG. 6 (NR12-b, NR32-b, and NR33-b).

With continued reference to FIG. 6, it can be seen that with both factors present, all three candidate enzymes began producing UDP-rhamnose as early as the 3-hour mark (panel A). More UDP-rhamnose was produced when the reaction was extended for longer reaction time (panels B and C in FIG. 6). With the one-step cofactor addition approach, NR32-a showed the highest activity for UDP-rhamnose production (0.57 g/L UDP-Rh at 18 hr) among the three candidate enzymes. In this first set of experiment (a), it was observed that NR12-a has high UDP-glucose 4,6-dehydratase (DH) activity but very low UDP-4-keto-6-deoxy-glucose 3,5-epimerase and UDP-4-keto-rhamnose 4-keto-reductase (ER) activity, as evidenced by the high level of (almost complete) conversion from UDP-glucose (UDPG) to UDP-4-keto-6-deoxy-glucose (UDP4K6G). These results indicated that all three enzymes are trifunctional UDP-rhamnose synthase for the bioconversion of UDP-glucose to UDP-rhamnose.

In addition, the inventors also found that a two-step cofactor addition approach can enhance the conversion efficiency, indicating that later NADPH addition can avoid the negative feedback regulation of UDP-rhamnose on DH enzyme. In the two-step cofactors addition process, NAD⁺ was added in the initial reaction and NADPH was added in the reaction after 3 hr. As shown in FIG. 6, both of NR32 (NR32-b) and NR33 (NR33-b) has higher UDP-rhamnose production than one step reaction (NR32-a and NR33-a). NR32-b has the highest activity for producing UDP-Rh, reaching 1.1 g/L UDP-Rh at 18 hr (panel C). Consistent with the results of the first set of experiment, NR12-b showed high DH activity but very low ER activity, as evidenced by the high level of conversion from UDP-glucose to UDP4K6G, but very little UDP-rhamnose production.

These results showed that a two-step cofactor addition approach may be used to enhance the conversion efficiency from UDP-glucose to UDP-rhamnose.

### Example 2

### Two Step Addition of Cofactors

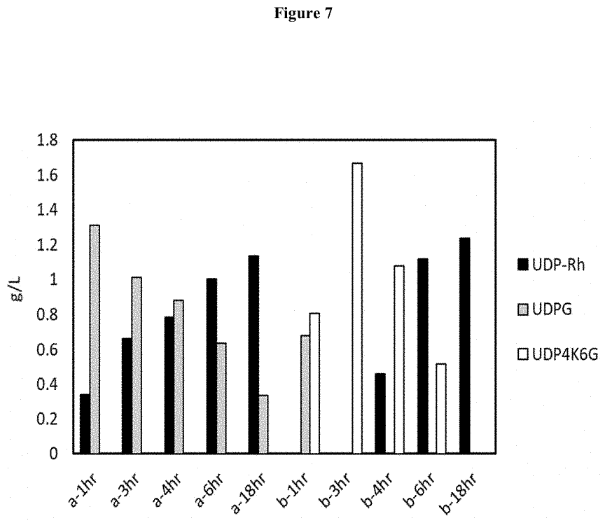

FIG. 7 shows how the two-step cofactor addition approach according to the present disclosure can enhance the conversion efficiency for UDP-rhamnose production in the reaction involving trifunctional enzyme NRF1. In the two-step reaction (b-1 hr, b-3 hr, b-4 hr, b-6 hr, b-18 hr), NAD⁺ was added in the initial reaction. UDPG substrate was fully converted to UDP-4-keto-6-deoxyglucose by DH activity at 3 hr (b-3 hr). Then NADPH was added in the reaction and UDP-4-keto-6-deoxyglucose was shown to have been fully converted to UDP-rhamnose at 18 hr (b-18 hr). In the one-step reaction (a-1 hr, a-3 hr, a-4 hr, a-6 hr, a-18 hr), both NAD+ and NADPH were added in the initial reaction and UDPG was converted to UDP-rhamnose incompletely, supporting that UDP-rhamnose has a negative feedback effect on DH activity as reported. The level of UDP-glucose (UDPG), UDP-4-keto-6-deoxyglucose (UDP4K6G), and UDP-rhamnose (UDP-Rh) were measured after 1 hour, 3 hours, 4 hours, 6 hours, and 18 hours under both approaches (“a” denoting the one-step approach, and “b” denoting the two-step approach).

### Example 3

### Optimization of One-Pot Multi-Enzyme System for In Vitro Synthesis of UDP-Rhamnose

Sucrose synthase (SUS) can break down a molecule of sucrose to yield a molecule of fructose and a molecule of glucose. In addition, SUS can transfer one glucose to UDP to form UDP-glucose. Therefore, by including sucrose, UDP, and SUS in the feedstock, the required UDP-glucose component in the UDP-rhamnose synthesis pathway disclosed herein can be replenished in the presence of sucrose synthase.

In addition, NADPH is a critical cofactor of ER activity. In the course of the ER-catalyzed reaction, NADPH is oxidized to NADP⁺. By incorporating an NADP⁺-dependent oxidation reaction as part of the UDP-rhamnose synthesis disclosed herein, NADPH can be regenerated. Exemplary NADP⁺-dependent oxidation reactions include the oxidation of malate into pyruvate, the oxidation of formate into CO₂, and the oxidation of phosphite into phosphate. By including malate, formate, or phosphite and the corresponding enzyme (MaeB, FDH, and PTDH, respectively) that can catalyze each of these oxidation reactions in the feedstock, NADPH is continuously regenerated, further optimizing the overall UDP-rhamnose production yield. Tables 1 provides information about the sequences of various enzymes.

In this example, six different experiments were performed with varying combinations of starting materials in a one-pot multi-enzyme reaction system using the two-step cofactor addition approach. Table 2 provides the composition of six different reaction systems tested int this experiment.

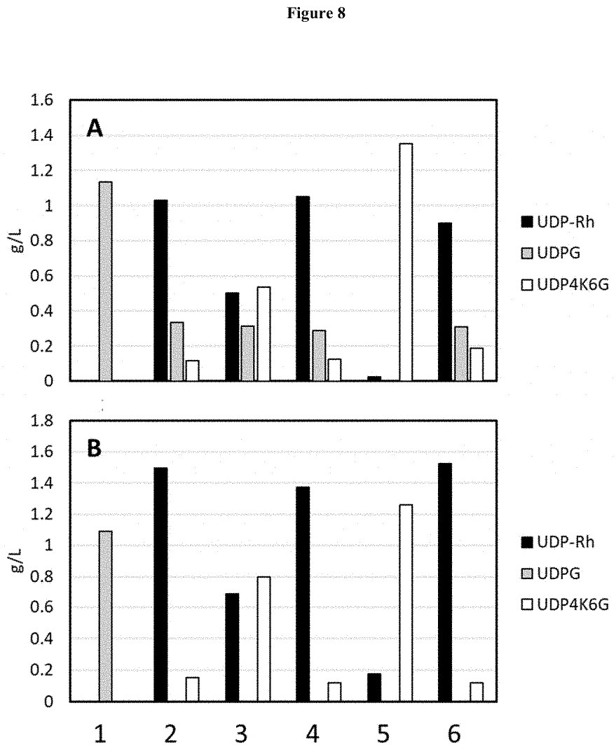

In each of the six systems, UDP-glucose was not included. Instead, UDP, sucrose and SUS were provided to produce the required UDP-glucose. Referring to FIG. 8, the results from System 1 show that UDP-glucose was produced, confirming that SUS can fully convert UDP to UDP-glucose. By providing a sucrose synthase enzyme (SUS) together with an RHM enzyme (e.g., NRF1), UDP-rhamnose can be produced using UDP as the substrate (System 2).

The experiments also confirmed the effect of NADPH regeneration in UDP-rhamnose production. With continued reference to FIG. 8, by adding the MaeB enzyme and malate in a reaction system that contains a low amount of NADPH (System 4), a high level of UDP-rhamnose can still be obtained, confirming the regeneration of NADPH. By comparison, in System 3 in which the same amount of NADPH was included but the MaeB enzyme was absent, a much lower amount of UDP-Rh was produced. Similarly, in reaction systems containing low amounts of NADP⁺ (Systems 5 and 6), provided that the MaeB enzyme is present, the added NADP⁺ can be converted to NADPH by MaeB and continually regenerate NADPH for UDP-rhamnose production (System 6). The amount of UDP-rhamnose obtained in System 6 with only 1 mM of NADP⁺ was comparable to the amount obtained in System 2 with 3 mM of NADPH. By comparison, in System 5 which includes no NADPH and no MaeB, hardly any of the UDP4K6G was converted into UDP-rhamnose. As mentioned above, the malate/MaeB system can be substituted with other NADP⁺-dependent oxidation systems such as formate/FDH and phosphite/PTDH.

### Example 4

### Enzymatic Activity Screening of UDP-glucose 4,6-dehydratase

UDP-glucose 4,6-dehydratase (DH) can catalyze the enzymatic reaction for bioconversion of UDP-glucose (UDPG) to UDP-4-keto-6-deoxy-glucose (UDP4K6G). In order to identify specific DH enzymes, enzyme candidates were selected based on polygenetic and Blast analysis.

Full length DNA fragments of all candidate DH genes were commercially synthesized. Almost all codons of the cDNA were changed to those preferred for E. coli (Gene Universal, DE). The synthesized DNA was cloned into a bacterial expression vector pETite N-His SUMO Kan Vector (Lucigen).

Each expression construct was transformed into E. coli BL21 (DE3), which was subsequently grown in LB media containing 50 μg/mL kanamycin at 37° C. until reaching an OD600 of 0.8-1.0. Protein expression was induced by addition of 1 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer, pH 7.2, 25 ug/ml lysozyme, 5 ug/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged DH recombinant polypeptides were eluted by equilibration buffer containing 250 mM imidazole.

The purified candidate DH recombinant polypeptides were assayed for UDP-4-keto-6-deoxy-glucose synthesis by using UDPG as substrate. Typically, the recombinant polypeptide (20 μg) was tested in a 200 μl in vitro reaction system. The reaction system contains 50 mM potassium phosphate buffer, pH 8.0, 3 mM MgCl₂, 3 mM UDPG, 3 mM NAD⁺ and 1 mM DTT. The reaction was performed at 30-37° C. and reaction was terminated by adding 200 μL chloroform. The samples were extracted with same volume chloroform by vertex for 10 mins. The supernatant was collected for high-performance liquid chromatography (HPLC) analysis after 10 mins centrifugation.

HPLC analysis was then performed using an Agilent 1200 system (Agilent Technologies, CA), including a quaternary pump, a temperature-controlled column compartment, an auto sampler and a UV absorbance detector. The chromatographic separation was performed using Dionex Carbo PA10 column (4×120 mm, Thermo Scientific) with mobile phase delivered at a flow rate of 1 ml/min. The mobile phase was H₂O (MPA) and 700 mM ammonium acetic (pH 5.2) (MPB). The gradient concentration of MPB was programmed for sample analysis. The detection wavelength used in the HPLC analysis was 261 nm.

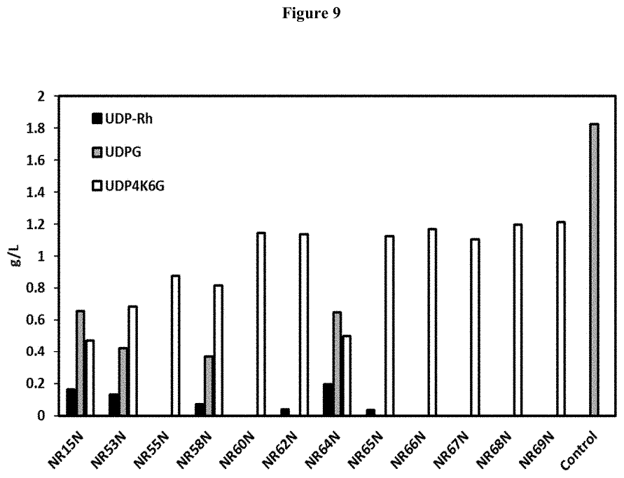

After activity screening, 12 novel DH enzymes were identified for bioconversion of UDPG to UDP4K6G (Table 1). As shown in FIG. 9, DH enzymes show various levels of enzymatic activity for UDP4K6G production. In addition, six candidates (NR15N, NR53N, NR58N, NR62N, NR64N, and NR65N) also show low enzymatic activity for UDP-rhamnose production, indicating these enzymes may have trifunctional activity (RHM) for UDP-L-rhamnose synthesis from UDPG.

### Example 5

### Enzymatic Activity Screening of Bifunctional UDP-4-keto-6-deoxy-Glucose 3,5-epimerase/UDP-4-keto Rhamnose 4-keto Reductase

Bifunctional UDP-4-keto-6-deoxy-glucose 3,5-epimerase/UDP-4-keto rhamnose 4-keto reductase (ER) enzymes can convert UDP-4-keto-6-deoxy-glucose to UDP-β-L-rhamnose. In order to identify specific ER enzymes, certain enzyme candidates were selected based on polygenetic and Blast analysis.

Full length DNA fragments of all candidate ER genes were commercially synthesized. Almost all codons of the cDNA were changed to those preferred for E. coli (Gene Universal, DE). The synthesized DNA was cloned into a bacterial expression vector pETite N-His SUMO Kan Vector (Lucigen).

Each expression construct was transformed into E. coli BL21 (DE3), which was subsequently grown in LB media containing 50 μg/mL kanamycin at 37° C. until reaching an OD600 of 0.8-1.0. Protein expression was induced by addition of 1 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer, pH 7.2, 25 ug/ml lysozyme, 5 ug/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged ER recombinant polypeptides were eluted by equilibration buffer containing 250 mM imidazole.

The purified candidate ER recombinant polypeptides were assayed for UDP-rhamnose synthesis by using UDP-4-keto-6-deoxy-glucose (UDP4K6G) as substrate. Typically, the recombinant polypeptide (20 μg) was tested in a 200 μl in vitro reaction system. The reaction system contains 50 mM potassium phosphate buffer, pH 8.0, 3 mM MgCl₂, 3 mM UDP-4-keto-6-deoxy glucose, 3 mM NADPH and 1 mM DTT. The reaction was performed at 30-37° C. and reaction was terminated by adding 200 μL chloroform. The samples were extracted with same volume chloroform by vertex for 10 mins. The supernatant was collected for high-performance liquid chromatography (HPLC) analysis after 10 mins centrifugation.

HPLC analysis was then performed using an Agilent 1200 system (Agilent Technologies, CA), including a quaternary pump, a temperature-controlled column compartment, an auto sampler and a UV absorbance detector. The chromatographic separation was performed using Dionex Carbo PA10 column (4×120 mm, Thermo Scientific) with mobile phase delivered at a flow rate of 1 ml/min. The mobile phase was H₂O (MPA) and 700 mM ammonium acetic (pH 5.2) (MPB). The gradient concentration of MPB was programmed for sample analysis. The detection wavelength used in the HPLC analysis was 261 nm.

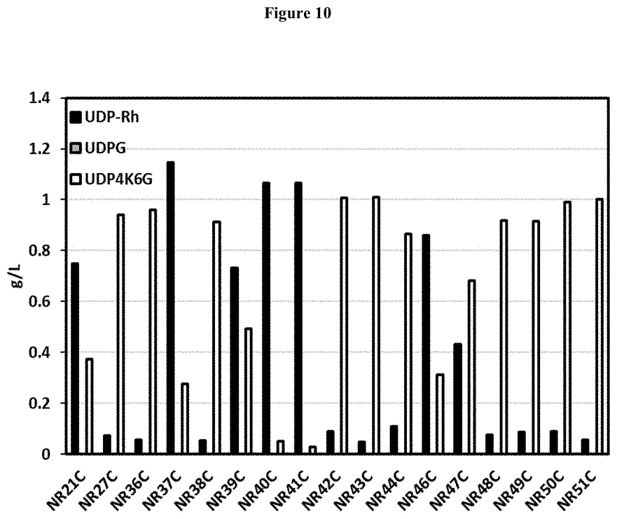

After activity screening, 17 novel ER enzymes were identified for bioconversion of UDP-4-keto-6-deoxy-glucose to UDP-L-rhamnose (Table 1). As shown in FIG. 10, the seventeen candidates show various levels of enzymatic activity for UDP-L-rhamnose production. Among the 17 enzyme candidates, the following enzymes show high ER activity: NR21C, NR37C, NR40C, NR41C, and NR46C.

### Example 6

### Identify Novel Fusion Enzyme for UDP-Rhamnose Production

Construction of fusion enzymes by recombinant DNA technology could be useful in obtaining new trifunctional enzymes with UDP-rhamnose synthase activity. However, the fusion of two functional enzymes do not necessarily provide an active fusion enzyme having the activity of both enzyme components. In addition, suitable linkers are often identified only empirically.

Based on extensive screening of various DH and ER enzyme candidates as well as N-terminal and C-terminal domains of trifunctional RHM enzymes, a series of fusion enzymes with specific DH and ER domains were identified and screened.

After such further screening, six fusion enzymes were found to have trifunctional activity for bioconversion of UDP-glucose to UDP-rhamnose (Table 3).

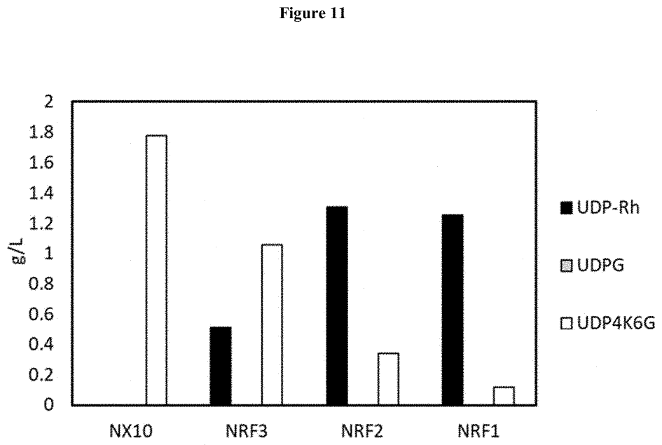

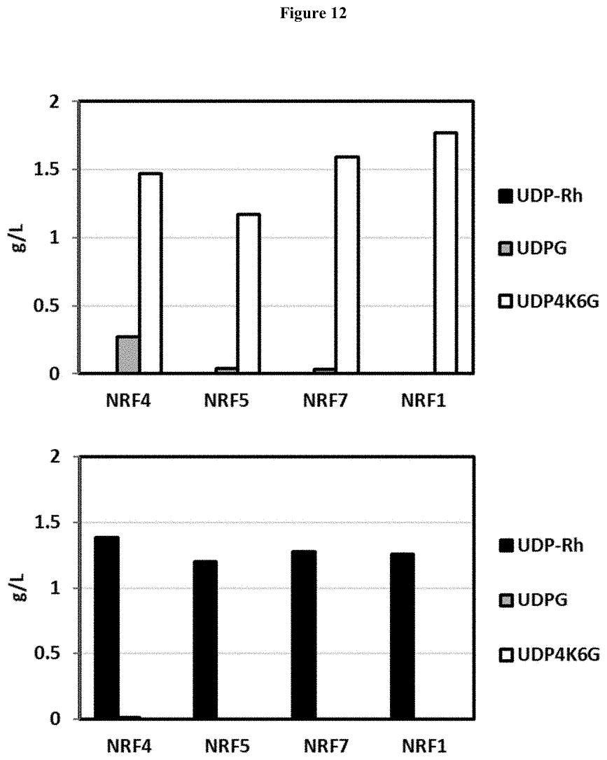

Specifically, five of these fusions enzymes are based on high activity DH enzyme NX10 fused with different ER enzymes (NX5C, NX13, NR5C, NR40C, and NR41C), namely, NRF1 (NX10-NX5C), NRF2 (NX10-NX13), NRF3 (NX10-NR5C), NRF4 (NX10-NR40C), and NRF5 (NX10-NR41C). An additional fusion enzyme with trifunctional activity, NRF7 (NR66N-NR41C), is based on high activity DH enzyme NR66N fused with high activity ER enzyme NR41C. As shown in FIG. 11, NX10 signal enzyme can completely convert UDP-glucose to UDP-4-keto-6-deoxyglucose (UDP4K6G). Meanwhile, FIGS. 11 and 12 show that fusion enzymes NRF1, NRF2, NRF3, NRF4, NRF5, and NRF7 all have trifunctional activity for UDP-rhamnose synthesis in the two steps cofactor addition reaction where NADPH was added after 3 hr reaction. Notably, NRF1, NRF2, NRF4, NRF5 and NRF7 fusion enzymes have higher enzymatic activity than NRF3.

### Example 7

### Combination of UDP-Rhamnose and Steviol Glycoside Production

As described in commonly-owned International Application No. PCT/US2019/021876, now published as WO2019/178116A1, the inventors have identified various UDP-rhamnosyltransferases (1,2 RhaT) for the biosynthesis of rhamnose-containing steviol glycosides such as Reb J and Reb N. Specifically, Reb J and Reb N can be synthesized from Reb A and UDP-rhamnose.

Referring to FIG. 2, by coupling the biosynthetic pathway for the production of UDP-rhamnose disclosed herein with the biosynthetic pathway for the production of Reb J/N from Reb A as disclosed in International Application No. PCT/US2019/021876, a one-pot multi-enzyme reaction system is provided for the in vitro bioconversion of Reb J/N from Reb A and UDP-glucose.

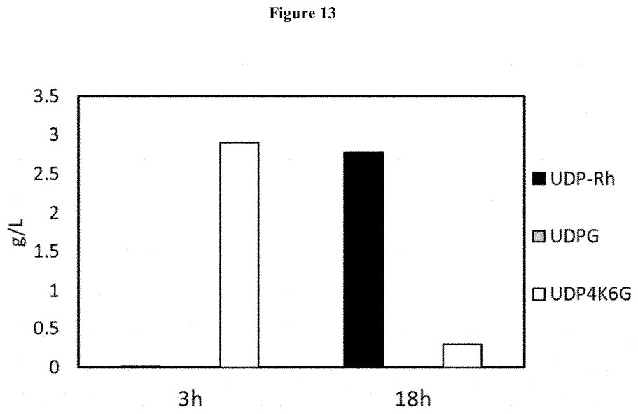

In the first step, UDP-glucose was converted to UDP-rhamnose by an RHM enzyme such as NRF1 (SEQ ID NO: 9) through a two-step cofactor addition process. UDP-glucose (6 mM) was fully converted to UDP-4-keto-6-deoxyglucose at 3 hour (FIG. 13). Subsequently, 0.5 mM NADP⁺ and an NADPH-regeneration system (e.g., MaeB enzyme and malate) was added in the reaction, converting UDP-4-keto-6-deoxyglucose to UDP-rhamnose. Referring to FIG. 13, almost 3 g/L of UDP-Rh was obtained after 18 hours.

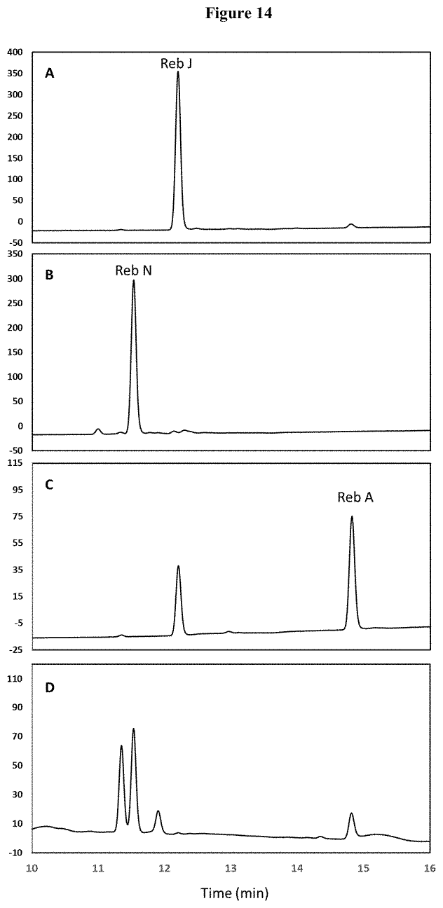

In the second step, Reb A and a UDP-rhamnosyltransferase such as EUCP1 (SEQ ID NO: 23) were added into the reaction system. The UDP-rhamnosyltransferase enzyme transfers one rhamnose moiety from UDP-rhamnose to the C-2′ of the 19-O-glucose of the Reb A substrate, thereby converting Reb A to Reb J. The level of Reb J was measured at 22-hr. The activity of EUCP1 was confirmed by HPLC, which shows the presence of Reb J (FIG. 14, panel C). UDP was released as a side product.

In the third step, a UDP-glycosyltransferase enzyme such as CP1 (SEQ ID NO: 25), a sucrose synthase enzyme such as SUS (SEQ ID NO: 15) and sucrose was added into the reaction mixture. The SUS enzyme catalyzed the reaction that produces UDP-glucose and fructose from UDP and sucrose. The CP1 enzyme catalyzed the conversion of Reb J to Reb N, specifically, by transferring one glucosyl moiety from UDP-glucose to the C-3′ of the 19-O-glucose of Reb J to produce Reb N and UDP. The UDP produced was converted back to UDP-glucose by the SUS enzyme in the presence of sucrose for UDP-rhamnose and Reb N production. HPLC analysis confirmed that Reb N was produced from Reb J at 25-hr (FIG. 14, panel D).

Based on these results, and referring again to FIG. 2, the present one-pot multi-enzyme reaction can be summarized as follows. In the reaction, UDP-glucose can be converted to UDP-rhamnose by a UDP-rhamnose synthase (e.g., NRF1) via a two-step cofactor addition process. A UDP-rhamnosyltransferase (e.g., EUCP1) can be used to transfer one rhamnose moiety from UDP-rhamnose to the C-2′ of the 19-O-glucose of Reb A to produce Reb J and UDP. The produced UDP can be converted to UDP-glucose by a SUS enzyme using sucrose as a source of glucose. A UDP-glycosyltransferase enzyme (e.g., CP1) can be used to transfer one glucosyl moiety from UDPG to the C-3′ of the 19-O-glucose of Reb J to produce Reb N and UDP. The produced UDP can be converted back to UDP-glucose by the SUS enzyme for UDP-rhamnose and Reb N production.

TABLE 1
Sequence Information

| Seq. ID No. | Sequence Detail |
|---|---|
| 1 | NR12 - Predicted amino acid sequence of UDP-rhamnose synthase |
| | from Ricinus communis. |
| 2 | NR12 - Predicted nucleic acid sequence of UDP-rhamnose synthase |
| | from Ricinus communis. |
| 3 | NR32 - Predicted amino acid sequence of UDP-rhamnose synthase |
| | from Ceratopteris thalictroides. |
| 4 | NR32 - Predicted nucleic acid sequence of UDP-rhamnose synthase |
| | from Ceratopteris thalictroides. |
| 5 | NR33 - Predicted amino acid sequence of UDP-rhamnose synthase |
| | from Azolla filiculoides. |
| 6 | NR33 - Predicted nucleic acid sequence of UDP-rhamnose synthase |
| | from Azolla filiculoides. |
| 7 | NX10 - Amino acid sequence of UDP-glucose 4,6-dehydratase |
| | (DH) [Botrytis cinerea] |
| 8 | NX10 - Nucleic acid sequence of UDP-glucose 4,6-dehydratase |
| | (DH) [Botrytis cinerea] |
| 9 | Amino acid sequence of Fusion enzyme NRF1 |
| 10 | Nucleic acid sequence of Fusion enzyme NRF1 |
| 11 | Amino acid sequence of Fusion enzyme NRF2 |
| 12 | Nucleic acid sequence of Fusion enzyme NRF2 |
| 13 | Amino acid sequence of Fusion enzyme NRF3 |
| 14 | Nucleic acid sequence of Fusion enzyme NRF3 |
| 15 | Amino acid sequence of Sucrose synthase SUS [Arabidopsis |
| | thaliana] |
| 16 | Nucleic Acid sequence of Sucrose synthase SUS [Arabidopsis |
| | thaliana] |
| 17 | Amino acid sequence of Malic enzyme MaeB [Escherichia coli] |
| 18 | Nucleic acid sequence of Malic enzyme MaeB [Escherichia coli] |
| 19 | Amino acid sequence of Formate dehydrogenase FDH [Candida |
| | boidinii] |
| 20 | Nucleic acid sequence of Formate dehydrogenase FDH [Candida |
| | boidinii] |
| 21 | Amino acid sequence of Phosphite dehydrogenase PTDH |
| | [Pseudomonas stutzeri] |
| 22 | Nucleic acid sequence of Phosphite dehydrogenase PTDH |
| | [Pseudomonas stutzeri] |
| 23 | EUCP1 - Amino acid sequence of UDP-rhamnosyltransferase (1,2 |
| | RhaT) |
| 24 | EUCP1 - Nucleic acid sequence of UDP-rhamnosyltransferase (1,2 |
| | RhaT) |
| 25 | CP1 - Amino acid sequence of UDP-glycosyltransferase (UGT) |
| 26 | CP1 - Nucleic acid sequence of UDP-glycosyltransferase (UGT) |
| 27 | NR55N - Amino acid sequence of UDP-glucose 4,6-dehydratase (DH) |
| | Acrostichum aureum |
| 28 | NR55N - Nucleic acid sequence of UDP-glucose 4,6-dehydratase (DH) |
| | Acrostichum aureum |
| 29 | NR60N - Amino acid sequence of UDP-glucose 4,6-dehydratase (DH) |
| | Ettlia oleoabundans |
| 30 | NR60N - Nucleic acid sequence of UDP-glucose 4,6-dehydratase (DH) |
| | Ettlia oleoabundans |
| 31 | NR66N - Amino acid sequence of UDP-glucose 4,6-dehydratase (DH) |
| | Volvox carteri |
| 32 | NR66N - Nucleic acid sequence of UDP-glucose 4,6-dehydratase (DH) |
| | Volvox carteri |
| 33 | NR67N - Amino acid sequence of UDP-glucose 4,6-dehydratase (DH) |
| | Chlamydomonas reinhardtii |
| 34 | NR67N - Nucleic acid sequence of UDP-glucose 4,6-dehydratase (DH) |
| | Chlamydomonas reinhardtii |
| 35 | NR68N - Amino acid sequence of UDP-glucose 4,6-dehydratase (DH) |
| | Oophila amblystomatis |
| 36 | NR68N - Nucleic acid sequence of UDP-glucose 4,6-dehydratase (DH) |
| | Oophila amblystomatis |
| 37 | NR69N - Amino acid sequence of UDP-glucose 4,6-dehydratase (DH) |
| | Dunaliella primolecta |
| 38 | NR69N - Nucleic acid sequence of UDP-glucose 4,6-dehydratase (DH) |
| | Dunaliella primolecta |
| 39 | NR15N - Amino acid sequence of RHM Ostreococcus lucimarinus |
| 40 | NR15N - Nucleic acid sequence of RHM Ostreococcus lucimarinus |
| 41 | NR53N - Amino acid sequence of RHM Nannochloropsis oceanica |
| 42 | NR53N - Nucleic acid sequence of RHM Nannochloropsis oceanica |
| 43 | NR58N - Amino acid sequence of RHM Ulva lactuca |
| 44 | NR58N - Nucleic acid sequence of RHM Ulva lactuca |
| 45 | NR62N - Amino acid sequence of RHM Golenkinia longispicula |
| 46 | NR62N - Nucleic acid sequence of RHM Golenkinia longispicula |
| 47 | NR65N - Amino acid sequence of RHM Tetraselmis subcordiformis |
| 48 | NR65N - Nucleic acid sequence of RHM Tetraselmis subcordiformis |
| 49 | NR21C - Amino acid sequence of bifunctional UDP-4-keto-6-deoxyl- glucose |
| | 3,5-epimerase/UDP-4-keto rhamnose 4-keto reductase (ER) |
| | Physcomitrella patens subsp. Patens |
| 50 | NR21C - Nucleic acid sequence of bifunctional UDP-4-keto-6-deoxyl- |
| | glucose 3,5-epimerase/UDP-4-keto rhamnose 4-keto reductase (ER) |
| | Physcomitrella patens subsp. Patens |
| 51 | NR27C - Amino acid sequence of bifunctional UDP-4-keto-6-deoxyl- |
| | glucose 3,5-epimerase/UDP-4-keto rhamnose 4-keto reductase (ER) |
| | Pyricularia oryzae |
| 52 | NR27C - Nucleic acid sequence of bifunctional UDP-4-keto-6-deoxyl- |
| | glucose 3,5-epimerase/UDP-4-keto rhamnose 4-keto reductase (ER) |
| | Pyricularia oryzae |
| 53 | NR36C - Amino acid sequence of bifunctional UDP-4-keto-6-deoxyl- |
| | glucose 3,5-epimerase/UDP-4-keto rhamnose 4-keto reductase (ER) |
| | Nannochloropsis oceanica |
| 54 | NR36C - Nucleic acid sequence of bifunctional UDP-4-keto-6-deoxyl- |
| | glucose 3,5-epimerase/UDP-4-keto rhamnose 4-keto reductase (ER) |
| | Nannochloropsis oceanica |
| 55 | NR37C - Amino acid sequence of bifunctional UDP-4-keto-6-deoxyl- |
| | glucose 3,5-epimerase/UDP-4-keto rhamnose 4-keto reductase (ER) Ulva |
| | lactuca |
| 56 | NR37C - Nucleic acid sequence of bifunctional UDP-4-keto-6-deoxyl- |
| | glucose 3,5-epimerase/UDP-4-keto rhamnose 4-keto reductase (ER) Ulva |
| | lactuca |
| 57 | NR38C - Amino acid sequence of bifunctional UDP-4-keto-6-deoxyl- |
| | glucose 3,5-epimerase/UDP-4-keto rhamnose 4-keto reductase (ER) |
| | Tetraselmis cordiformis |
| 58 | NR38C - Nucleic acid sequence of bifunctional UDP-4-keto-6-deoxyl- |
| | glucose 3,5-epimerase/UDP-4-keto rhamnose 4-keto reductase (ER) |
| | Tetraselmis cordiformis |
| 59 | NR39C - Amino acid sequence of bifunctional UDP-4-keto-6-deoxyl- |
| | glucose3,5-epimerase/UDP-4-keto rhamnose 4-keto reductase (ER) |
| | Tetraselmis subcordiformis |
| 60 | NR39C - Nucleic acid sequence of bifunctional UDP-4-keto-6-deoxyl- |
| | glucose3,5-epimerase/UDP-4-keto rhamnose 4-keto reductase (ER) |
| | Tetraselmis subcordiformis |
| 61 | NR40C - Amino acid sequence of bifunctional UDP-4-keto-6-deoxyl- |
| | glucose 3,5-epimerase/UDP-4-keto rhamnose 4-keto reductase (ER) |
| | Chlorella sorokiniana |
| 62 | NR40C - Nucleic acid sequence of bifunctional UDP-4-keto-6-deoxyl- |
| | glucose 3,5-epimerase/UDP-4-keto rhamnose 4-keto reductase (ER) |
| | Chlorella sorokiniana |
| 63 | NR41C - Amino acid sequence of bifunctional UDP-4-keto-6-deoxyl- |
| | glucose 3,5-epimerase/UDP-4-keto rhamnose 4-keto reductase (ER) |
| | Chlamydomonas moewusii |
| 64 | NR41C - Nucleic acid sequence of bifunctional UDP-4-keto-6-deoxyl- |
| | glucose 3,5-epimerase/UDP-4-keto rhamnose 4-keto reductase (ER) |
| | Chlamydomonas moewusii |
| 65 | NR42C - Amino Acid sequence of bifunctional UDP-4-keto-6-deoxyl- |
| | glucose 3,5-epimerase/UDP-4-keto rhamnose 4-keto reductase (ER) |
| | Golenkinia longispicula |
| 66 | NR42C - Nucleic Acid sequence of bifunctional UDP-4-keto-6-deoxyl- |
| | glucose 3,5-epimerase/UDP-4-keto rhamnose 4-keto reductase (ER) |
| | Golenkinia longispicula |
| 67 | NR43C - Amino acid sequence of bifunctional UDP-4-keto-6-deoxyl- |
| | glucose 3,5-epimerase/UDP-4-keto rhamnose 4-keto reductase (ER) |
| | Chlamydomonas reinhardtii |
| 68 | NR43C - Nucleic acid sequence of bifunctional UDP-4-keto-6-deoxyl- |
| | glucose 3,5-epimerase/UDP-4-keto rhamnose 4-keto reductase (ER) |
| | Chlamydomonas reinhardtii |
| 69 | NR44C - Amino acid sequence of bifunctional UDP-4-keto-6-deoxyl- |
| | glucose 3,5-epimerase/UDP-4-keto rhamnose 4-keto reductase (ER) |
| | Chromochloris zofingiensis |
| 70 | NR44C - Nucleic acid sequence of bifunctional UDP-4-keto-6-deoxyl- |
| | glucose 3,5-epimerase/UDP-4-keto rhamnose 4-keto reductase (ER) |
| | Chromochloris zofingiensis |
| 71 | NR46C - Amino acid sequence of bifunctional UDP-4-keto-6-deoxyl- |
| | glucose 3,5-epimerase/UDP-4-keto rhamnose 4-keto reductase (ER) |
| | Dunaliella primolecta |
| 72 | NR46C - Nucleic acid sequence of bifunctional UDP-4-keto-6-deoxyl- |
| | glucose 3,5-epimerase/UDP-4-keto rhamnose 4-keto reductase (ER) |
| | Dunaliella primolecta |
| 73 | NR47C - Amino acid sequence of bifunctional UDP-4-keto-6-deoxyl- |
| | glucose 3,5-epimerase/UDP-4-keto rhamnose 4-keto reductase (ER) |
| | Pavlova lutheri |
| 74 | NR47C - Nucleic acid sequence of bifunctional UDP-4-keto-6-deoxyl- |
| | glucose 3,5-epimerase/UDP-4-keto rhamnose 4-keto reductase (ER) |
| | Pavlova lutheri |
| 75 | NR48C - Amino acid sequence of bifunctional UDP-4-keto-6-deoxyl- |
| | glucose 3,5-epimerase/UDP-4-keto rhamnose 4-keto reductase (ER) |
| | Nitella mirabilis |
| 76 | NR48C - Nucleic acid sequence of bifunctional UDP-4-keto-6-deoxyl- |
| | glucose 3,5-epimerase/UDP-4-keto rhamnose 4-keto reductase (ER) |
| | Nitella mirabilis |
| 77 | NR49C - Amino acid sequence of bifunctional UDP-4-keto-6-deoxyl- |
| | glucose 3,5-epimerase/UDP-4-keto rhamnose 4-keto reductase (ER) |
| | Marchantia polymorpha |
| 78 | NR49C - Nucleic acid sequence of bifunctional UDP-4-keto-6-deoxyl- |
| | glucose 3,5-epimerase/UDP-4-keto rhamnose 4-keto reductase (ER) |
| | Marchantia polymorpha |
| 79 | NR50C - Amino acid sequence of bifunctional UDP-4-keto-6-deoxyl- |
| | glucose 3,5-epimerase/UDP-4-keto rhamnose 4-keto reductase (ER) |
| | Selaginella moellendorffii |
| 80 | NR50C - Nucleic acid sequence of bifunctional UDP-4-keto-6-deoxyl- |
| | glucose 3,5-epimerase/UDP-4-keto rhamnose 4-keto reductase (ER) |
| | Selaginella moellendorffii |
| 81 | NR51C - Amino acid sequence of bifunctional UDP-4-keto-6-deoxyl- |
| | glucose 3,5-epimerase/UDP-4-keto rhamnose 4-keto reductase (ER) |
| | Bryum argenteum var argenteum |
| 82 | NR51C - Nucleic acid sequence of bifunctional UDP-4-keto-6-deoxyl- |
| | glucose 3,5-epimerase/UDP-4-keto rhamnose 4-keto reductase (ER) |
| | Bryum argenteum var argenteum |
| 83 | NRF4 - Amino acid sequence of RHM, fusion enzyme |
| 84 | NRF4 - Nucleic acid sequence of RHM, fusion enzyme |
| 85 | NRF5 - Amino acid sequence of RHM, fusion enzyme |
| 86 | NRF5 - Nucleic acid sequence of RHM, fusion enzyme |
| 87 | NRF7- Amino acid sequence of RHM, fusion enzyme |
| 88 | NRF7 - Nucleic acid sequence of RHM, fusion enzyme |
| 89 | NR64N - Amino acid sequence of RHM from Tetraselmis cordiformis |
| 90 | NR64N - Nucleic acid sequence of RHM from Tetraselmis cordiformis |
| 91 | NX5C - Amino acid sequence of bifunctional UDP-4-keto-6-deoxyl- |
| | glucose 3,5-epimerase/UDP-4-keto rhamnose 4-keto reductase (ER) |
| | Arabidopsis thaliana |
| 92 | NX5C - Nucleic acid sequence of bifunctional UDP-4-keto-6-deoxyl- |
| | glucose 3,5-epimerase/UDP-4-keto rhamnose 4-keto reductase (ER) |
| | Arabidopsis thaliana |
| 93 | NX13 - Amino acid sequence of bifunctional UDP-4-keto-6-deoxyl- |
| | glucose 3,5-epimerase/UDP-4-keto rhamnose 4-keto reductase (ER) |
| | Pyricularia oryzae |
| 94 | NX13 - Nucleic acid sequence of bifunctional UDP-4-keto-6-deoxyl- |
| | glucose 3,5-epimerase/UDP-4-keto rhamnose 4-keto reductase (ER) |
| | Pyricularia oryzae |
| 95 | NR5C - Amino acid sequence of bifunctional UDP-4-keto-6-deoxyl- |
| | glucose 3,5-epimerase/UDP-4-keto rhamnose 4-keto reductase (ER) |
| | Citrus clementina |
| 96 | NR5C - Nucleic acid sequence of bifunctional UDP-4-keto-6-deoxyl- |
| | glucose 3,5-epimerase/UDP-4-keto rhamnose 4-keto reductase (ER) |
| | Citrus clementina |
| 97 | EU11 - Amino acid sequence of 1,2-rhamnosyltransferase - Oryza sativa |
| 98 | EU11 - Nucleotide sequence of 1,2-rhamnosyltransferase - Oryza sativa |
| 99 | HV1 - Amino acid sequence of 1,2-rhamnosyltransferase - Hordeum |
| | vulgare |
| 100 | HV1 - cleotide sequence of 1,2-rhamnosyltransferase - Hordeum vulgare |
| 101 | UGT2E-B - Artificial Sequence - Amino acid sequence of 1,2- |
| | rhamnosyltransferase |
| 102 | UGT2E-B - Artificial Sequence - Nucleotide sequence of 1,2- |
| | rhamnosyltransferase |
| 103 | NX114 Amino acid sequence of 1,2-rhamnosyltransferase - Oryza |
| | brachyantha |
| 104 | NX114 Nucleic acid sequence of 1,2-rhamnosyltransferase - Oryza |
| | brachyantha |
| 105 | CP2 - Artificial Sequence - Amino acid sequence of UDP- |
| | glycosyltransferase |
| 106 | CP2 - Artificial Sequence - Nucleotide sequence of UDP-glycosyltransferase |
| 107 | UGT76G1 - Amino acid acid sequence of UDP-glycosyltransferase - Stevia |
| | rebaudiana |
| 108 | UGT76G1 - Nucleic acid sequence of UDP-glycosyltransferase - Stevia |
| | rebaudiana |
| 109 | GS - Amino acid sequence of fusion enzyme - UDP-glycosyltransferase + |
| | Sucrose Synthase |
| 110 | Artificial Sequence - Nucleic acid sequence of fusion enzyme - UDP- |
| | glycosyltransferase + Sucrose Synthase |

TABLE 2
One-pot multi-enzyme in vitro synthesis of UDP-rhamnose.

| Reaction No. | 1 | 2 | 3 | 4 | 5 | 6 |
|---|---|---|---|---|---|---|
| PB pH 8.0 | 50 mM | 50 mM | 50 mM | 50 mM | 50 mM | 50 mM |
| UDP | 3 mM | 3 mM | 3 mM | 3 mm | 3 mm | 3 mM |
| Sucrose | 250 mM | 250 mM | 250 mM | 250 mM | 250 mM | 250 mM |
| NAD+ | 3 mM | 3 mM | 3 mM | 3 mM | 3 mM | 3 mM |
| NADPH | 3 mM | 3 mM | 1 mM | 1 mM | 0 | 0 |
| NADP+ | 0 | 0 | 0 | 0 | 1 Mm | 1 mM |
| DTT | 1 mM | 1 mM | 1 mM | 1 mm | 1 mM | 1 mM |
| NRF1 | 0 | 0.2 g/l | 0.2 g/l | 0.2 g/l | 0.2 g/l | 0.2 g/l |
| MaeB | 0 | 0 | 0 | 0.1 g/l | 0 | 0.1 g/l |
| SUS | 0.2 g/l | 0.2 g/l | 0.2 g/l | 0.2 g/l | 02. g/l | 0.2 g/l |
| Malate | 5 mM | 5 mM | 5 mM | 5 mM | 5 mm | 5 mM |
| MgCl2 | 3 mM | 3 mM | 3 mM | 3 mM | 3 mM | 3 mM |

TABLE 3
Amino acid sequence organization in fusion enzymes

| Fusion | N-terminal end | Linker amino | C-terminal end |
|---|---|---|---|
| enzyme | (SEQ ID NO.) | acid sequence | (SEQ ID NO.) |
| NRF1 | NX10 | GSG | NX5C |
| | (SEQ ID NO. 7) | (Gly-Ser-Gly) | (SEQ ID NO. 91) |
| NRF2 | NX10 | GSG | NX13 |
| | (SEQ ID NO. 7) | (Gly-Ser-Gly) | (SEQ ID NO. 93) |
| NRF3 | NX10 | GSG | NR5C |
| | (SEQ ID NO. 7) | (Gly-Ser-Gly) | (SEQ ID NO. 95) |
| NRF4 | NX10 | GSG | NR40C |
| | (SEQ ID NO. 7) | (Gly-Ser-Gly) | (SEQ ID NO. 61) |
| NRF5 | NX10 | GSG | NR41C |
| | (SEQ ID NO. 7) | (Gly-Ser-Gly) | (SEQ ID NO. 63) |
| NRF7 | NR66N | GSG | NR41C |
| | (SEQ ID No. 31) | (Gly-Ser-Gly) | (SEQ ID NO. 63) |

1. A biosynthetic method of preparing uridine diphosphate-rhamnose (UDP-rhamnose) from uridine diphosphate-glucose (UDP-glucose), the method comprising incubating UDP-glucose with one or more recombinant polypeptides having UDP-rhamnose synthase activity in the presence of NAD+ and a source of NADPH for a sufficient time to produce UDP-rhamnose.

2. The method of claim 1, wherein the one or more recombinant polypeptides comprise a first recombinant polypeptide that is a trifunctional enzyme having UDP-glucose 4,6-dehydratase, UDP-4-keto-6-deoxy-glucose 3,5-epimerase, and UDP-4-keto-rhamnose 4-keto-reductase activities.

3. The method of claim 1, wherein the one or more recombinant polypeptides comprise a first recombinant polypeptide that is a fusion enzyme comprising a first domain having UDP-glucose 4,6-dehydratase activity and a second domain having UDP-4-keto-6-deoxy-glucose 3,5-epimerase and UDP-4-keto-rhamnose 4-keto-reductase activities.

4. The method of claim 1, wherein the one or more recombinant polypeptides comprise a first recombinant polypeptide having UDP-glucose 4,6-dehydratase activity and a second recombinant polypeptide having UDP-4-keto-6-deoxy-glucose 3,5-epimerase and UDP-4-keto-rhamnose 4-keto-reductase activities.

5. The method of claim 3, wherein the first domain of the fusion enzyme comprises an amino acid sequence having at least 80% sequence identity to SEQ ID NO: 7 or SEQ ID NO: 31.

6. The method of claim 5, wherein the second domain of the fusion enzyme comprises an amino acid sequence having at least 80% sequence identity to SEQ ID NO: 91, SEQ ID NO: 93, SEQ ID NO: 95, SEQ ID NO: 61, or SEQ ID NO: 63.

7. The method of claim 6, wherein the fusion enzyme comprises an amino acid sequence having at least 80% sequence identity to SEQ ID NO: 9, SEQ ID NO: 11, or SEQ ID NO: 13, SEQ ID NO: 83, SEQ ID NO: 85, or SEQ ID NO: 87.

8.-12. (canceled)

13. The method of claim 1, wherein the one or more recombinant polypeptides comprise a first recombinant polypeptide that is a fusion polypeptide coded by a nucleotide resulting from the fusion between a first nucleotide coding for a UDP-glucose 4,6-dehydratase enzyme and a second nucleotide coding for a bifunctional enzyme having UDP-4-keto-6-deoxy-glucose 3,5-epimerase and UDP-4-keto-rhamnose 4-keto-reductase activities.

14.-17. (canceled)

18. The method of claim 2, wherein the trifunctional enzyme comprises an amino acid sequence having at least 80% sequence identity to SEQ ID NO: 3 or SEQ ID NO: 5.

19. The method of claim 4, wherein the first recombinant polypeptide comprises an amino acid sequence having at least 80% sequence identity to SEQ ID NO: 7, SEQ ID NO: 29, SEQ ID NO: 31, SEQ ID NO: 33, SEQ ID NO: 35, or SEQ ID NO: 37, and/or wherein the second recombinant polypeptide comprises an amino acid sequence having at least 80% sequence identity to SEQ ID NO: 49, SEQ ID NO: 55, SEQ ID NO: 61, SEQ ID NO: 63, or SEQ ID NO: 71.

20. (canceled)

21. The method of claim 1, comprising expressing said one or more recombinant polypeptides in a transformed cellular system.

22.-29. (canceled)

30. The method of claim 1, wherein the uridine diphosphate-glucose and the one or more recombinant polypeptides are incubated with sucrose and a third recombinant polypeptide having sucrose synthase activity.

31. (canceled)

32. A biosynthetic method of preparing a steviol glycoside composition comprising at least one rhamnose-containing steviol glycoside, the method comprising:
(a) incubating a substrate selected from the group consisting of sucrose, uridine diphosphate and uridine diphosphate-glucose, with one or more recombinant polypeptides having UDP-rhamnose synthase activity in the presence of NAD+ and a source of NADPH to produce uridine diphosphate-rhamnose; and
(b) reacting the uridine diphosphate-rhamnose with a steviol glycoside substrate in the presence of a recombinant polypeptide having rhamnosyltransferase activity, so that a rhamnose moiety is coupled to the steviol glycoside substrate to produce at least one rhamnose-containing steviol glycoside.

33. The method of claim 32, wherein the steviol glycoside substrate is rebaudioside A.

34. The method of claim 32, wherein the steviol glycoside composition comprises rebaudioside N, rebaudioside J, or both.

35. The method of claim 32, further comprises reacting the rhamnose-containing steviol glycoside in the presence of a recombinant polypeptide having glycosyltransferase activity, so that a glucose moiety is coupled to the rhamnose-containing steviol glycoside.

36. The method of claim 32, wherein the substrate comprises uridine diphosphate-glucose.

37. The method of claim 36, wherein the uridine diphosphate-glucose substrate is provided in situ by reacting sucrose and uridine diphosphate in the presence of a sucrose synthase.

38. A nucleic acid comprising a sequence encoding a polypeptide comprising an amino acid sequence having at least 99% identity to SEQ ID NO: 9, SEQ ID NO: 11, SEQ ID NO. 13, SEQ ID NO. 83, SEQ ID NO. 85 or SEQ ID NO. 87.

39. A cell comprising the nucleic acid of claim 38.

40.-42. (canceled)


SEQUENCE LISTING (110 sequences)

SEQ ID NO: 1 - protein, 369 aa, Ricinus communis
MSSNHAPYEPKKILITGAAGFIASHVTNRLIRNYPDYKIVALDKLDYCSSLRNLTPCRSSPNFKFVKGDIASADLVNHLLIAEDIDTIMHFAAQTHVDNSFGNSFEFTTNNIYGTHVLLEACKVTKKIKRFIHVSTDEVYGETDMETDIGNPEASQLLPTNPYSATKAGAEMLVMAYHRSYGLPTITTRGNNVYGPNQYPEKLIPKFIILAMKGEQLPIHGNGSNVRSYLHCEDVAEAFDVILHKGAIGHVYNIGTKKERRVLDVAEDICRLFRLDAKKAIRFVQDRPFNDQRYFLDDQKLKKLGWQERTPWEEGLKMTMEWYTKNPNWWGDVSAALHPHPRISMVVHSNDDSWLLEDGCAKEGDNNSS

SEQ ID NO: 2 - DNA, 1110 bp, Ricinus communis
atgagcagtaatcatgcaccgtatgaaccgaaaaagattctgattaccggtgccgcaggttttattgccagccatgttaccaatcgtctgattcgtaattatccggattataaaatcgtggccctggataaactggattattgtagcagcctgcgcaatctgaccccgtgccgcagtagtccgaattttaaatttgttaaaggcgatatcgccagcgcagatttggttaatcatctgctgattgcagaagatattgataccattatgcattttgcagcccagacccatgtggataatagctttggcaatagctttgagtttactaccaataatatctacggtacccatgttctgctggaagcatgtaaagttaccaaaaagattaagcgtttcatccatgtgagcaccgatgaagtttatggcgaaaccgatatggaaaccgatattggcaatccggaagcaagtcagctgctgccgaccaatccgtatagcgcaaccaaagcaggcgcagaaatgctggttatggcatatcatcgtagctatggcctgccgaccattaccacccgcggtaataatgtgtatggtccgaatcagtatccggaaaaactgattccgaaattcattattctggcaatgaaaggtgaacagctgccgattcatggcaatggtagtaatgttcgtagttatctgcattgcgaagatgttgcagaagcatttgatgtgattctgcataaaggtgccattggccatgtttataatattggtaccaaaaaagagcgccgtgttctggatgttgcagaggatatttgtcgtctgtttcgtctggatgcaaaaaaggcaattcgttttgtgcaggatcgtccgtttaatgatcagcgctattttctggatgatcagaaactgaaaaagctgggctggcaggaacgcaccccgtgggaagaaggcctgaaaatgaccatggaatggtataccaaaaatccgaattggtggggcgatgtgagtgccgcactgcatccgcatccgcgtattagcatggttgttcatagcaatgatgatagctggctgctggaagatggttgcgccaaagaaggtgacaataatagcagctaa

SEQ ID NO: 3 - protein, 672 aa, Ceratopteris thalictroides
MAANYYTPKNILITGAAGFIASHVANRLVRNYPQYKIVVLDKLDYCSNLKNLGPSRASKNFKFVQGDIGSADLVNYLLKTEAIDTIMHFAAQTHVDNSFGNSFEFTKNNVYGTHVLLEACKVTGTIRRFIHVSTDEVYGETEANAIVGNHEASQLLPTNPYSATKAGAEMLVMAYGRSYGLPFITTRGNNVYGPNQFPEKLIPKFILLAMQGKPLPIHGDGSNVRSYLFCEDVAEAFEVVLHKGEVGNVYNIGTTRERRVLDVAKDICKLFELDPKKVIEFVDNRPFNDQRYFLDDKKLKDLGWEERTPWEEGLRKTMEWYSKNPDWWGDVSGALVPHPRMLAIGGLDRTACDLPNHTPLEVHPNGTMDNPKVKAPLKFLIYGRTGWIGGLLGDACKKQGIEYEYGSGRLENRSSLEADIERVKPTHVLNAAGLTGRPNVDWCESHKTETVSVNVVGTLSLADVCLQHDLLLVNFATGCIFEYDDSHPLGSGIGFREEDTPNFTGSFYSKTKAMVEELLKNYSNVCTLRVRMPISSDLSNPRNFITKITRYQKVVDIPNSMTVLDEMVPIAIEMAKRNLTGIWNFTNPGVVSHNEILEMYRKYIDPKFQWINFSLEEQAKVIIAPRSNNELDASKLQREFPGLLSIKDSLLKYVFEVNKNLRLMKKMVEPLS

SEQ ID NO: 4 - DNA, 2019 bp, Ceratopteris thalictroides
atggcagccaattattataccccgaaaaatattctgatcaccggtgccgccggctttattgcaagccatgttgcaaatcgtctggttcgtaattatccgcagtataaaattgtggttctggataaactggattattgtagcaatctgaaaaacctgggtccgagtcgtgcaagcaaaaattttaaatttgtgcagggtgacatcggcagcgccgatctggtgaattatctgctgaaaaccgaagccattgataccattatgcattttgccgcccagacccatgttgataatagctttggcaatagttttgagtttactaaaaacaacgtgtacggcacccatgtgctgctggaagcctgtaaagtgaccggtaccattcgccgttttattcatgtgagtaccgatgaagtgtatggtgaaaccgaagccaatgcaattgttggtaatcatgaagcaagtcagctgctgccgaccaatccgtatagcgcaaccaaagcaggtgccgaaatgctggttatggcctatggtcgtagttatggcctgccgtttattaccacccgtggtaataatgtttatggcccgaatcagtttccggaaaaactgattccgaaattcattctgctggcaatgcagggtaaaccgctgccgattcatggcgatggcagtaatgtgcgtagttatctgttttgtgaagatgtggcagaagcatttgaagttgttctgcataaaggcgaagtgggtaatgtttataatattggcaccacccgcgaacgccgcgtgctggatgttgcaaaagatatttgcaaactgtttgaactggatccgaaaaaagtgattgaatttgtggataatcgcccgtttaatgatcagcgctattttctggatgataaaaaactgaaagacctgggctgggaagaacgtaccccgtgggaagaaggtctgcgcaaaaccatggaatggtatagcaaaaatccggattggtggggtgacgttagcggtgcactggtgccgcatccgcgtatgctggcaattggtggtctggatcgcaccgcatgtgatctgccgaatcataccccgctggaagtgcatccgaatggtaccatggataatccgaaagttaaagccccgctgaaatttctgatctatggtcgcaccggctggattggtggcctgctgggcgatgcatgcaaaaaacagggcattgaatatgaatatggtagcggtcgtctggaaaatcgcagcagcctggaagccgatattgaacgcgttaaaccgacccatgtgttaaatgccgccggtctgaccggccgcccgaatgttgattggtgcgaaagccataaaaccgaaaccgtgagtgttaatgttgttggtaccctgagcctggccgatgtttgtctgcaacatgatctgctgctggttaattttgcaaccggctgcatttttgaatatgatgatagccatccgctgggcagtggcattggctttcgcgaagaagataccccgaattttaccggtagcttttatagtaaaaccaaagccatggttgaagaactgctgaaaaattatagtaacgtttgtaccctgcgtgtgcgtatgccgattagcagtgatctgagtaatccgcgcaattttattaccaaaattacccgctatcagaaagtggtggatattccgaatagcatgaccgttctggatgaaatggttccgattgccattgaaatggccaaacgcaatctgaccggtatttggaattttaccaatccgggtgttgtgagccataatgaaattctggaaatgtatcgcaaatacattgatccgaaatttcagtggattaatttcagtctggaagaacaggcaaaagtgattattgcaccgcgtagtaataatgaactggatgcaagtaaactgcaacgcgaatttccgggtctgctgagcattaaggatagcctgctgaaatatgtttttgaagttaataagaacctgcgtctgatgaaaaagatggtggaaccgctgagctaa

SEQ ID NO: 5 - protein, 683 aa, Azolla filiculoides
MANNASYTPKNILITGAAGFIASHVANRLVASYPQYKIVVLDKLDYCSNLKNLIPSRSSKNFKFVRGDIGSADLVNYLLITEGIDTIMHFAAQTHVDNSFGNSLEFTKNNVYGTHVLLEACKVTGNIRRFIHVSTDEVYGETEADAMVGNHEASQLLPTNPYSATKAGAEMLVMAYGRSYGLPVITTRGNNVYGPNQFPEKLIPKFILLAMQGRPLPIHGDGSNVRSYLYCEDVAEAFEVVLHKGEVGHVYNIGTTRERTVLDVAKDICKLFKLDAEKLIQFVENRPFNDQRYFLDDKKLKELGWEERTSWEDGLSKTMEWYLKNPGWWGDVSGALVPHPRMLAIGCVEKLDLPLDKSTNDDTLDASLGSRTSNNGSYPSLHESSMAKTSNGSSISEEYKFLIYGRTGWIGGLLGKICKEQGIEYHYGSGRLENREQLELDIERVKPTHVFNAAGVTGRPNVDWCESHKTETIRSNVVGTLTLADVCLAHGLLLVNFATGCIFEYDGKHPLGSGVGFLEEDTPNFTGSFYSKTKAMVEDLLKNYDNVCTLRVRMPISSDLENPRNFITKITRYQKVVNIPNSMTVLDEMLPIAVEMAKRRLTGIWNFTNPGVVSHNEILEMYKEFIDTGFKYSNFTLEEQAKVIVAPRSNNELDASKLKKEFPELLSIKDSLMKYVFEVNKKT

SEQ ID NO: 6 - DNA, 2052 bp, Azolla filiculoides;
atggcaaataacgccagctataccccgaaaaatattctgattaccggcgccgccggttttattgccagtcatgttgccaatcgcctggtggcaagctatccgcagtataaaattgtggtgctggataaactggattattgtagtaatctgaagaacctgattccgagtcgtagcagtaaaaattttaaatttgtgcgcggcgatattggtagcgcagatttggtgaattatctgctgattaccgaaggtattgataccattatgcattttgcagcacagacccatgttgataatagttttggtaatagcctggagtttactaaaaataatgtgtatggtacccacgtgctgctggaagcatgcaaagttaccggtaatattcgtcgctttattcatgttagtaccgatgaagtttacggcgaaaccgaagccgatgccatggtgggtaatcatgaagccagtcagctgctgccgaccaatccgtatagcgcaaccaaagcaggcgccgaaatgctggttatggcctatggccgcagctatggcctgccggttattaccacccgtggtaataatgtgtacggtccgaatcagtttccggaaaaactgattccgaaattcattctgctggcaatgcagggtcgcccgctgccgattcatggtgacggtagcaatgtgcgtagttatctgtattgtgaagatgttgcagaagcatttgaagtggttctgcataaaggcgaagttggccatgtttataatattggtaccacccgcgaacgtaccgtgctggatgtggcaaaagatatttgcaaactgtttaaactggacgccgaaaaactgatccagtttgtggaaaatcgcccgtttaatgatcagcgttattttctggatgataaaaaactgaaggagctgggttgggaagaacgcaccagctgggaagatggtctgagtaaaaccatggaatggtatctgaaaaatccgggctggtggggtgacgttagcggtgccctggtgccgcatccgcgcatgctggcaattggttgtgtggaaaaactggatctgccgctggataaaagcaccaatgatgataccctggatgcaagtctgggtagtcgcaccagcaataatggcagttatccgagcctgcatgaaagtagtatggccaaaaccagcaatggtagtagtattagcgaagaatataagtttctgatctacggtcgtaccggctggattggcggtctgctgggcaaaatttgtaaagaacagggtattgaataccattatggtagtggccgtctggaaaatcgtgaacagctggaactggatattgaacgtgtgaaaccgacccatgtgtttaatgccgccggtgtgaccggccgcccgaatgttgattggtgtgaaagccataaaaccgaaaccattcgcagcaatgtggtgggtaccctgaccctggccgatgtgtgcctggcccatggcctgctgctggttaattttgccaccggttgcatttttgaatatgatggtaaacatccgctgggtagtggtgttggctttctggaagaagataccccgaattttaccggcagcttttatagtaaaaccaaagcaatggttgaggatctgctgaaaaattatgataatgtttgcaccctgcgcgttcgcatgccgattagtagtgatctggaaaatccgcgcaattttattaccaaaattacccgttatcagaaggtggttaatattccgaatagtatgaccgttctggatgaaatgctgccgattgcagttgaaatggcaaaacgtcgtctgaccggtatttggaattttaccaatccgggcgtggttagtcataatgaaattctggaaatgtacaaggagtttattgataccggttttaaatacagtaacttcaccctggaagaacaggccaaagttattgtggcaccgcgtagcaataatgaactggatgccagcaaactgaaaaaagaatttccggaactgctgagcattaaggatagcctgatgaaatatgttttcgaagttaataagaagacctaa

SEQ ID NO: 7 - protein, 431 aa, Botrytis cinerea; [sequence omitted]

SEQ ID NO: 8 - DNA, 1296 bp, Botrytis cinerea;
atggcagcaaatggtacaaccccgagcagcgcaaatgaagaacagaataaattctttgaggattttggcgtgtggaaagaagcaccgattctgattggtagcaccaaatttgaaccgctgccggatgttaaaaacattatgattaccggtggtgccggttttattgcatgttggctggttcgtcatctgaccctgacctatccggatgcatataacattgtgagcttcgataaactggattattgtgccagcctgaataatacccgtgcactgaatgataaacgcaactttagcttttatcacggcgatattaccaatccgagcgaagttgttgattgtctggaacgctataacatcgataccatctttcattttgcagcccagagccatgttgatctgagctttggtaatagctatgcatttacccataccaatgtttatggcacccatgttctgctggaaagcgcaaaaaaagttggcatcaaaaagttcatccacatcagcaccgatgaagtttatggtgaagtgaaagatgatgatgacgatttactggaaaccagcattctggcaccgaccaatccgtatgcagcaagcaaagcagcagcagaaatgctggtgcatagttatcagaaatcatttaaactgccggtgatgattgtgcgcagcaataatgtgtatggtccgcatcagtatccggaaaaaatcattccgaaattcagctgtctgctgcaacgtggtcagccggttgttctgcatggtgatggcaccccgacacgtcgttacctgtttgcgggtgatgcagcagatgcatttgataccattctgcataaaggcaccattggccagatttataacgttggtagctatgacgaaatcagcaatctgacactgtgtagcaaactgctgacatatctggatattccgcatagcacccaagaggaactgcataaatgggttaaacatacccaggatcgtccgtttaatgatcatcgttatgccgttgatggtacaaaactgcgtcagttaggttgggatcagaaaaccagctttgaaaatggtatggcagttaccgtggattggtataaacgttttggtgaacgttggtggggtgatattacaaaagttctgaccccgtttccgaccgttgcaggtagcaaagttgttggtgatgataataacaccgtcgaagaactgaaagaagagatggttattgacgccgatgataacatgattctgggcaaaaaacgtaaactgaatggtgttccgagcggtctggcacaggcagttgaagcataa

SEQ ID NO: 9 - protein, 730 aa, Artificial Sequence
  Synthetic polypeptide
MAANGTTPSSANEEQNKFFEDFGVWKEAPILIGSTKFEPLPDVKNIMITGGAGFIACWLVRHLTLTYPDAYNIVSFDKLDYCASLNNTRALNDKRNFSFYHGDITNPSEVVDCLERYNIDTIFHFAAQSHVDLSFGNSYAFTHTNVYGTHVLLESAKKVGIKKFIHISTDEVYGEVKDDDDDLLETSILAPTNPYAASKAAAEMLVHSYQKSFKLPVMIVRSNNVYGPHQYPEKIIPKFSCLLQRGQPVVLHGDGTPTRRYLFAGDAADAFDTILHKGTIGQIYNVGSYDEISNLTLCSKLLTYLDIPHSTQEELHKWVKHTQDRPFNDHRYAVDGTKLRQLGWDQKTSFENGMAVTVDWYKRFGERWWGDITKVLTPFPTVAGSKVVGDDNNTVEELKEEMVIDADDNMILGKKRKLNGVPSGLAQAVEAGSGQRSNGTPQKPSLKFLIYGKTGWIGGLLGKICDKQGIAYEYGKGRLEDRSSLLQDIQSVKPTHVFNSAGVTGRPNVDWCESHKTETIRANVAGTLTLADVCREHGLLMMNFATGCIFEYDDKHPEGSGIGFKEEDTPNFTGSFYSKTKAMVEELLKEYDNVCTLRVRMPISSDLNNPRNFITKISRYNKVVNIPNSMTVLDELLPISIEMAKRNLKGIWNFTNPGVVSHNEILEMYRDYINPEFKWANFTLEEQAKVIVAPRSNNEMDASKLKKEFPELLSIKESLIKYAYGPNKKT

SEQ ID NO: 10 - DNA, 2193 bp, Artificial Sequence
  Synthetic polynucleotide
atggcagcaaatggtacaaccccgagcagcgcaaatgaagaacagaataaattctttgaggattttggcgtgtggaaagaagcaccgattctgattggtagcaccaaatttgaaccgctgccggatgttaaaaacattatgattaccggtggtgccggttttattgcatgttggctggttcgtcatctgaccctgacctatccggatgcatataacattgtgagcttcgataaactggattattgtgccagcctgaataatacccgtgcactgaatgataaacgcaactttagcttttatcacggcgatattaccaatccgagcgaagttgttgattgtctggaacgctataacatcgataccatctttcattttgcagcccagagccatgttgatctgagctttggtaatagctatgcatttacccataccaatgtttatggcacccatgttctgctggaaagcgcaaaaaaagttggcatcaaaaagttcatccacatcagcaccgatgaagtttatggtgaagtgaaagatgatgatgacgatttactggaaaccagcattctggcaccgaccaatccgtatgcagcaagcaaagcagcagcagaaatgctggtgcatagttatcagaaatcatttaaactgccggtgatgattgtgcgcagcaataatgtgtatggtccgcatcagtatccggaaaaaatcattccgaaattcagctgtctgctgcaacgtggtcagccggttgttctgcatggtgatggcaccccgacacgtcgttacctgtttgcgggtgatgcagcagatgcatttgataccattctgcataaaggcaccattggccagatttataacgttggtagctatgacgaaatcagcaatctgacactgtgtagcaaactgctgacatatctggatattccgcatagcacccaagaggaactgcataaatgggttaaacatacccaggatcgtccgtttaatgatcatcgttatgccgttgatggtacaaaactgcgtcagttaggttgggatcagaaaaccagctttgaaaatggtatggcagttaccgtggattggtataaacgttttggtgaacgttggtggggtgatattacaaaagttctgaccccgtttccgaccgttgcaggtagcaaagttgttggtgatgataataacaccgtcgaagaactgaaagaagagatggttattgacgccgatgataacatgattctgggcaaaaaacgtaaactgaatggtgttccgagcggtctggcacaggcagttgaagcaggttctggtcagcgtagcaatggtacaccgcagaaaccgagcctgaaatttctgatttatggtaaaaccggttggattggtggtctgctgggtaaaatttgcgataaacagggtatcgcctatgaatatggtaaaggtcgtctggaagatcgtagcagcctgctgcaagatattcagagcgttaaaccgacgcatgtgtttaatagtgccggtgtgaccggtcgtccgaatgttgattggtgtgaaagccataaaaccgaaaccattcgtgcaaatgttgcaggtacactgaccctggcagatgtttgtcgtgaacatggtttactgatgatgaattttgccaccggctgcatctttgagtatgatgataaacatccggaaggtagcggtatcggttttaaagaagaagatacaccgaattttaccggcagcttttacagcaaaaccaaagcaatggttgaggaactgctgaaagaatatgataatgtttgtaccctgcgtgtgcgtatgccgattagcagcgacctgaataatccgcgtaactttattaccaaaatctcccgctataacaaagtggtgaatattccgaatagcatgaccgtactggatgaactgctgcctattagcattgaaatggcaaaacgtaacctgaaaggcatctggaactttaccaatccgggtgttgttagccataacgaaattctggaaatgtaccgcgattatatcaacccggaatttaagtgggccaattttacactggaagaacaggccaaagttattgttgcaccgcgtagtaataatgaaatggatgcaagcaaactgaagaaagagtttccagaactgctgtccattaaagaaagcctgatcaaatatgcgtacggtccgaacaaaaaaacctaa

SEQ ID NO: 11 - protein, 725 aa, Artificial Sequence
  Synthetic polypeptide
MAANGTTPSSANEEQNKFFEDFGVWKEAPILIGSTKFEPLPDVKNIMITGGAGFIACWLVRHLTLTYPDAYNIVSFDKLDYCASLNNTRALNDKRNFSFYHGDITNPSEVVDCLERYNIDTIFHFAAQSHVDLSFGNSYAFTHTNVYGTHVLLESAKKVGIKKFIHISTDEVYGEVKDDDDDLLETSILAPTNPYAASKAAAEMLVHSYQKSFKLPVMIVRSNNVYGPHQYPEKIIPKFSCLLQRGQPVVLHGDGTPTRRYLFAGDAADAFDTILHKGTIGQIYNVGSYDEISNLTLCSKLLTYLDIPHSTQEELHKWVKHTQDRPFNDHRYAVDGTKLRQLGWDQKTSFENGMAVTVDWYKRFGERWWGDITKVLTPFPTVAGSKVVGDDNNTVEELKEEMVIDADDNMILGKKRKLNGVPSGLAQAVEAGSGTNNRFLIWGGEGWVAGHLASILKSQGKDVYTTTVRMENREGVLAELEKVKPTHVLNCAGCTGRPNVDWCEDNKEATMRSNVIGTLNLTDACFQKGIHCTVFATGCIYQYDDAHPWDGPGFLETDKANFAGSFYSETKAHVEEVMKYYNNCLILRLRMPVSDDLHPRNFVTKIAKYDRVVDIPNSNTILHDLLPLSLAMAEHKDTGVYNFTNPGAISHNEVLTLFRDIVRPSFKWQNFSLEEQAKVIKAGRSNCKLDTTKLTEKAKEYGIEVPEIHEAYRQCFERMKKAGVQ

SEQ ID NO: 12 - DNA, 2178 bp, Artificial Sequence
  Synthetic polynucleotide
atggcagcaaatggtacaaccccgagcagcgcaaatgaagaacagaataaattctttgaggattttggcgtgtggaaagaagcaccgattctgattggtagcaccaaatttgaaccgctgccggatgttaaaaacattatgattaccggtggtgccggttttattgcatgttggctggttcgtcatctgaccctgacctatccggatgcatataacattgtgagcttcgataaactggattattgtgccagcctgaataatacccgtgcactgaatgataaacgcaactttagcttttatcacggcgatattaccaatccgagcgaagttgttgattgtctggaacgctataacatcgataccatctttcattttgcagcccagagccatgttgatctgagctttggtaatagctatgcatttacccataccaatgtttatggcacccatgttctgctggaaagcgcaaaaaaagttggcatcaaaaagttcatccacatcagcaccgatgaagtttatggtgaagtgaaagatgatgatgacgatttactggaaaccagcattctggcaccgaccaatccgtatgcagcaagcaaagcagcagcagaaatgctggtgcatagttatcagaaatcatttaaactgccggtgatgattgtgcgcagcaataatgtgtatggtccgcatcagtatccggaaaaaatcattccgaaattcagctgtctgctgcaacgtggtcagccggttgttctgcatggtgatggcaccccgacacgtcgttacctgtttgcgggtgatgcagcagatgcatttgataccattctgcataaaggcaccattggccagatttataacgttggtagctatgacgaaatcagcaatctgacactgtgtagcaaactgctgacatatctggatattccgcatagcacccaagaggaactgcataaatgggttaaacatacccaggatcgtccgtttaatgatcatcgttatgccgttgatggtacaaaactgcgtcagttaggttgggatcagaaaaccagctttgaaaatggtatggcagttaccgtggattggtataaacgttttggtgaacgttggtggggtgatattacaaaagttctgaccccgtttccgaccgttgcaggtagcaaagttgttggtgatgataataacaccgtcgaagaactgaaagaagagatggttattgacgccgatgataacatgattctgggcaaaaaacgtaaactgaatggtgttccgagcggtctggcacaggcagttgaagcaggttctggtaccaataaccgttttctgatttggggtggtgaaggttgggttgcaggtcatctggcaagcattctgaaaagccagggtaaagatgtttataccaccaccgttcgtatggaaaatcgtgaaggtgttctggcagaactggaaaaagttaaaccgacacatgttctgaattgtgcaggttgtaccggtcgtccgaatgttgattggtgtgaagataataaagaagccaccatgcgtagcaatgttattggcaccctgaatctgaccgatgcatgttttcagaaaggtattcattgtaccgtttttgccaccggttgcatctatcagtatgatgatgcacatccgtgggatggtccgggttttctggaaaccgataaagcaaattttgccggtagcttttacagcgaaaccaaagcacatgttgaagaggtgatgaagtattacaacaactgtctgattctgcgtctgcgtatgccggttagtgatgatctgcatccgcgtaattttgtgaccaaaatcgcaaaatatgatcgcgttgtggatattccgaatagcaataccattctgcatgatctgctgccgctgagcctggcaatggcagaacataaagataccggtgtttacaactttaccaatccgggtgcaattagccataatgaagttctgaccctgtttcgtgatattgttcgtccgagctttaagtggcagaatttttcactggaagaacaggccaaagttattaaagcaggtcgtagcaattgtaaactggataccaccaaactgaccgaaaaagccaaagaatatggtattgaagtgccggaaattcatgaagcatatcgtcagtgttttgaacgcatgaaaaaagccggtgttcagtaa

SEQ ID NO: 13 - protein, 729 aa, Artificial Sequence
  Synthetic polypeptide
MAANGTTPSSANEEQNKFFEDFGVWKEAPILIGSTKFEPLPDVKNIMITGGAGFIACWLVRHLTLTYPDAYNIVSFDKLDYCASLNNTRALNDKRNFSFYHGDITNPSEVVDCLERYNIDTIFHFAAQSHVDLSFGNSYAFTHTNVYGTHVLLESAKKVGIKKFIHISTDEVYGEVKDDDDDLLETSILAPTNPYAASKAAAEMLVHSYQKSFKLPVMIVRSNNVYGPHQYPEKIIPKFSCLLQRGQPVVLHGDGTPTRRYLFAGDAADAFDTILHKGTIGQIYNVGSYDEISNLTLCSKLLTYLDIPHSTQEELHKWVKHTQDRPFNDHRYAVDGTKLRQLGWDQKTSFENGMAVTVDWYKRFGERWWGDITKVLTPFPTVAGSKVVGDDNNTVEELKEEMVIDADDNMILGKKRKLNGVPSGLAQAVEAGSGSKCSSPRKPSMKFLIYGRTGWIGGLLGKLCEKEGIPFEYGKGRLEDRSSLIADVQSVKPTHVFNAAGVTGRPNVDWCESHKTDTIRTNVAGTLTLADVCREHGILMMNYATGCIFEYDAAHPEGSGIGYKEEDTPNFTGSFYSKTKAMVEELLKEYDNVCTLRVRMPISSDLNNPRNFITKISRYNKVVNIPNSMTVLDELLPISIEMAKRNLRGIWNFTNPGVVSHNEILEMYKKYINPEFKWVNFTLEEQAKVIVAPRSNNEMDASKLKKEFPELLSIKDSLIKYVFEPNKKT

SEQ ID NO: 14 - DNA, 2190 bp, Artificial Sequence
  Synthetic polynucleotide
atggcagcaaatggtacaaccccgagcagcgcaaatgaagaacagaataaattctttgaggattttggcgtgtggaaagaagcaccgattctgattggtagcaccaaatttgaaccgctgccggatgttaaaaacattatgattaccggtggtgccggttttattgcatgttggctggttcgtcatctgaccctgacctatccggatgcatataacattgtgagcttcgataaactggattattgtgccagcctgaataatacccgtgcactgaatgataaacgcaactttagcttttatcacggcgatattaccaatccgagcgaagttgttgattgtctggaacgctataacatcgataccatctttcattttgcagcccagagccatgttgatctgagctttggtaatagctatgcatttacccataccaatgtttatggcacccatgttctgctggaaagcgcaaaaaaagttggcatcaaaaagttcatccacatcagcaccgatgaagtttatggtgaagtgaaagatgatgatgacgatttactggaaaccagcattctggcaccgaccaatccgtatgcagcaagcaaagcagcagcagaaatgctggtgcatagttatcagaaatcatttaaactgccggtgatgattgtgcgcagcaataatgtgtatggtccgcatcagtatccggaaaaaatcattccgaaattcagctgtctgctgcaacgtggtcagccggttgttctgcatggtgatggcaccccgacacgtcgttacctgtttgcgggtgatgcagcagatgcatttgataccattctgcataaaggcaccattggccagatttataacgttggtagctatgacgaaatcagcaatctgacactgtgtagcaaactgctgacatatctggatattccgcatagcacccaagaggaactgcataaatgggttaaacatacccaggatcgtccgtttaatgatcatcgttatgccgttgatggtacaaaactgcgtcagttaggttgggatcagaaaaccagctttgaaaatggtatggcagttaccgtggattggtataaacgttttggtgaacgttggtggggtgatattacaaaagttctgaccccgtttccgaccgttgcaggtagcaaagttgttggtgatgataataacaccgtcgaagaactgaaagaagagatggttattgacgccgatgataacatgattctgggcaaaaaacgtaaactgaatggtgttccgagcggtctggcacaggcagttgaagcaggttctggtagcaaatgtagcagtccgcgtaaaccgagcatgaaatttctgatttatggtcgcaccggttggattggtggtctgctgggcaaactgtgtgaaaaagaaggtattccgtttgagtatggtaaaggtcgtctggaagatcgtagcagcctgattgcagatgttcagagcgttaaaccgactcatgtttttaatgcagccggtgtgaccggtcgtccgaacgttgattggtgtgaaagccataaaaccgataccattcgtaccaatgttgcaggtacactgaccctggcagatgtttgtcgtgaacatggcattctgatgatgaattatgccaccggttgcatctttgaatatgatgcagcacatccggaaggtagcggtattggttataaagaagaagataccccgaattttaccggcagcttttatagcaaaaccaaggcaatggttgaggaactgctgaaagaatatgataatgtttgtaccctgcgtgtgcgtatgccgattagcagcgacctgaataatccgcgtaactttattaccaaaatcagccgctataacaaagtggtgaatattccgaatagcatgaccgtactggatgaactgctgcctattagcattgaaatggcaaaacgtaatctgcgtggcatttggaactttaccaatccgggtgttgttagccataacgaaattctggaaatgtacaaaaaatacatcaacccggaatttaagtgggtgaactttacactggaagaacaggccaaagttattgttgcaccgcgtagcaataatgaaatggatgcaagcaaactgaagaaagagtttccagaactgctgtccattaaagacagcctgatcaaatatgtgttcgaaccgaacaaaaaaacctaa

SEQ ID NO: 15 - protein, 808 aa, Arabidopsis thaliana;
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 16 - DNA, 2427 bp, Arabidopsis thaliana;
atggcaaacgctgaacgtatgattacccgtgtccactcccaacgcgaacgcctgaacgaaaccctggtgtcggaacgcaacgaagttctggcactgctgagccgtgtggaagctaagggcaaaggtattctgcagcaaaaccagattatcgcggaatttgaagccctgccggaacaaacccgcaaaaagctggaaggcggtccgtttttcgatctgctgaaatctacgcaggaagcgatcgttctgccgccgtgggtcgcactggcagtgcgtccgcgtccgggcgtttgggaatatctgcgtgtcaacctgcatgcactggtggttgaagaactgcagccggctgaatttctgcacttcaaggaagaactggttgacggcgtcaaaaacggtaattttaccctggaactggattttgaaccgttcaatgccagtatcccgcgtccgacgctgcataaatatattggcaacggtgtggactttctgaatcgccatctgagcgcaaagctgttccacgataaagaatctctgctgccgctgctgaaattcctgcgtctgcatagtcaccagggcaagaacctgatgctgtccgaaaaaattcagaacctgaataccctgcaacacacgctgcgcaaggcggaagaatacctggccgaactgaaaagtgaaaccctgtacgaagaattcgaagcaaagttcgaagaaattggcctggaacgtggctggggtgacaatgctgaacgtgttctggatatgatccgtctgctgctggacctgctggaagcaccggacccgtgcaccctggaaacgtttctgggtcgcgtgccgatggttttcaacgtcgtgattctgtccccgcatggctattttgcacaggacaatgtgctgggttacccggataccggcggtcaggttgtctatattctggatcaagttcgtgcgctggaaattgaaatgctgcagcgcatcaagcagcaaggcctgaacatcaaaccgcgtattctgatcctgacccgtctgctgccggatgcagttggtaccacgtgcggtgaacgtctggaacgcgtctatgacagcgaatactgtgatattctgcgtgtcccgtttcgcaccgaaaagggtattgtgcgtaaatggatcagtcgcttcgaagtttggccgtatctggaaacctacacggaagatgcggccgtggaactgtccaaggaactgaatggcaaaccggacctgattatcggcaactatagcgatggtaatctggtcgcatctctgctggctcataaactgggtgtgacccagtgcacgattgcacacgctctggaaaagaccaaatatccggattcagacatctactggaaaaagctggatgacaaatatcatttttcgtgtcagttcaccgcggacatttttgccatgaaccacacggattttattatcaccagtacgttccaggaaatcgcgggctccaaagaaaccgtgggtcaatacgaatcacataccgccttcacgctgccgggcctgtatcgtgtggttcacggtatcgatgtttttgacccgaaattcaatattgtcagtccgggcgcggatatgtccatctattttccgtacaccgaagaaaagcgtcgcctgacgaaattccattcagaaattgaagaactgctgtactcggacgtggaaaacaaggaacacctgtgtgttctgaaagataaaaagaaaccgatcctgtttaccatggcccgtctggatcgcgtgaagaatctgtcaggcctggttgaatggtatggtaaaaacacgcgtctgcgcgaactggcaaatctggtcgtggttggcggtgaccgtcgcaaggaatcgaaagataacgaagaaaaggctgaaatgaagaaaatgtacgatctgatcgaagaatacaagctgaacggccagtttcgttggatcagctctcaaatggaccgtgtgcgcaatggcgaactgtatcgctacatttgcgataccaagggtgcgtttgttcagccggcactgtacgaagctttcggcctgaccgtcgtggaagccatgacgtgcggtctgccgacctttgcgacgtgtaaaggcggtccggccgaaattatcgtgcatggcaaatctggtttccatatcgatccgtatcacggtgatcaggcagctgacaccctggcggatttctttacgaagtgtaaagaagacccgtcacactgggatgaaatttcgaagggcggtctgcaacgtatcgaagaaaaatatacctggcagatttacagccaacgcctgctgaccctgacgggcgtctacggtttttggaaacatgtgtctaatctggatcgcctggaagcccgtcgctatctggaaatgttttacgcactgaagtatcgcccgctggcacaagccgttccgctggcacaggacgactaa

SEQ ID NO: 17 - protein, 759 aa, Escherichia coli;
MDDQLKQSALDFHEFPVPGKIQVSPTKPLATQRDLALAYSPGVAAPCLEIEKDPLKAYKYTARGNLVAVISNGTAVLGLGNIGALAGKPVMEGKGVLFKKFAGIDVFDIEVDELDPDKFIEVVAALEPTFGGINLEDIKAPECFYIEQKLRERMNIPVFHDDQHGTAIISTAAILNGLRVVEKNISDVRMVVSGAGAAAIACMNLLVALGLQKHNIVVCDSKGVIYQGREPNMAETKAAYAVVDDGKRTLDDVIEGADIFLGCSGPKVLTQEMVKKMARAPMILALANPEPEILPPLAKEVRPDAIICTGRSDYPNQVNNVLCFPFIFRGALDVGATAINEEMKLAAVRAIAELAHAEQSEVVASAYGDQDLSFGPEYIIPKPFDPRLIVKIAPAVAKAAMESGVATRPIADFDVYIDKLTEFVYKTNLFMKPIFSQARKAPKRVVLPEGEEARVLHATQELVTLGLAKPILIGRPNVIEMRIQKLGLQIKAGVDFEIVNNESDPRFKEYWTEYFQIMKRRGVTQEQAQRALISNPTVIGAIMVQRGEADAMICGTVGDYHEHFSVVKNVFGYRDGVHTAGAMNALLLPSGNTFIADTYVNDEPDAEELAEITLMAAETVRRFGIEPRVALLSHSNFGSSDCPSSSKMRQALELVRERAPELMIDGEMHGDAALVEAIRNDRMPDSSLKGSANILVMPNMEAARISYNLLRVSSSEGVTVGPVLMGVAKPVHVLTPIASVRRIVNMVALAVVEAQTQPL

SEQ ID NO: 18 - DNA, 2280 bp, Escherichia coli;
atggatgaccagttaaaacaaagtgcacttgatttccatgaatttccagttccagggaaaatccaggtttctccaaccaagcctctggcaacacagcgcgatctggcgctggcctactcaccaggcgttgccgcaccttgtcttgaaatcgaaaaagacccgttaaaagcctacaaatataccgcccgaggtaacctggtggcggtgatctctaacggtacggcggtgctggggttaggcaacattggcgcgctggcaggcaaaccggtgatggaaggcaagggcgttctgtttaagaaattcgccgggattgatgtatttgacattgaagttgacgaactcgacccggacaaatttattgaagttgtcgccgcgctcgaaccaaccttcggcggcatcaacctcgaagatattaaagcgccagaatgtttctatattgaacagaaactgcgcgagcggatgaatattccggtattccacgacgatcagcacggcacggcaattatcagcactgccgccatcctcaacggcttgcgcgtggtggagaaaaacatctccgacgtgcggatggtggtttccggcgcgggtgccgcagcaatcgcctgtatgaacctgctggtagcgctgggtctgcaaaaacataacatcgtggtttgcgattcaaaaggcgttatctatcagggccgtgagccaaacatggcggaaaccaaagccgcgtatgcggtggtggatgacggcaaacgtaccctcgatgatgtgattgaaggcgcggatattttcctgggctgttccggcccgaaagtgctgacccaggaaatggtgaagaaaatggctcgtgcgccaatgatcctggcgctggcgaacccggaaccggaaattctgccgccgctggcgaaagaagtgcgtccggatgccatcatttgcaccggtcgttctgactatccgaaccaggtgaacaacgtcctgtgcttcccgttcatcttccgtggcgcgctggacgttggcgcaaccgccatcaacgaagagatgaaactggcggcggtacgtgcgattgcagaactcgcccatgcggaacagagcgaagtggtggcttcagcgtatggcgatcaggatctgagctttggtccggaatacatcattccaaaaccgtttgatccgcgcttgatcgttaagatcgctcctgcggtcgctaaagccgcgatggagtcgggcgtggcgactcgtccgattgctgatttcgacgtctacatcgacaagctgactgagttcgtttacaaaaccaacctgtttatgaagccgattttctcccaggctcgcaaagcgccgaagcgcgttgttctgccggaaggggaagaggcgcgcgttctgcatgccactcaggaactggtaacgctgggactggcgaaaccgatccttatcggtcgtccgaacgtgatcgaaatgcgcattcagaaactgggcttgcagatcaaagcgggcgttgattttgagatcgtcaataacgaatccgatccgcgctttaaagagtactggaccgaatacttccagatcatgaagcgtcgcggcgtcactcaggaacaggcgcagcgggcgctgatcagtaacccgacagtgatcggcgcgatcatggttcagcgtggggaagccgatgcaatgatttgcggtacggtgggtgattatcatgaacattttagcgtggtgaaaaatgtctttggttatcgcgatggcgttcacaccgcaggtgccatgaacgcgctgctgctgccgagtggtaacacctttattgccgatacctatgttaatgatgaaccggatgcagaagagctggcggagatcaccttgatggcggcagaaactgtccgtcgttttggtattgagccgcgcgttgctttgttgtcgcactccaactttggttcttctgactgcccgtcgtcgagcaaaatgcgtcaggcgctggaactggtcagggaacgtgcaccagaactgatgattgatggtgaaatgcacggcgatgcagcgctggtggaagcgattcgcaacgaccgtatgccggacagctctttgaaaggttccgccaatattctggtgatgccgaacatggaagctgcccgcattagttacaacttactgcgtgtttccagctcggaaggtgtgactgtcggcccggtgctgatgggtgtggcgaaaccggttcacgtgttaacgccgatcgcatcggtgcgtcgtatcgtcaacatggtggcgctggccgtggtagaagcgcaaacccaaccgctgtaa

SEQ ID NO: 19 - protein, 364 aa, Candida boidinii;
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLVPFNPKELLYYQHQALPKDAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAQGTKNILESFFTGKFDYRPQDIILLNGEYVTKAYGKHDKK

SEQ ID NO: 20 - DNA, 1095 bp, Candida boidinii;
atgaagatcgttttagtcttatatgatgctggtaaacacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactaccagcatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaaacacgataagaaataa

SEQ ID NO: 21 - protein, 336 aa, Pseudomonas stutzeri;
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKANPAAD

SEQ ID NO: 22 - DNA, 1014 bp, Pseudomonas stutzeri;
atgctgccgaaactcgttataactcaccgagtacacgaagagatcctgcaactgctggcgccacattgcgagctgatcaccaaccagaccgacagcacgctgacgcgcgaggaaattctgcgccgctgccgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagattgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaaccctgccgcagattgataa

SEQ ID NO: 23 - protein, 462 aa, Artificial Sequence
  Synthetic polypeptide
MGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTK

SEQ ID NO: 24 - DNA, 1389 bp, Artificial Sequence
  Synthetic polynucleotide
atgggtagctcgggcatgtccctggcggaacgcttttcgctgacgctgagtcgctcatccctggttgttggtcgcagttgtgttgaatttgaaccggaaaccgttccgctgctgtctacgctgcgcggcaaaccgattaccttcctgggtctgatgccgccgctgcatgaaggccgtcgcgaagatggtgaagacgccacggtgcgttggctggatgctcagccggcgaaatcggtggtttatgtcgcactgggcagcgaagtgccgctgggtgtcgaaaaagtgcacgaactggccctgggcctggaactggcaggcacccgctttctgtgggcactgcgtaaaccgacgggcgttagcgatgctgacctgctgccggcgggtttcgaagaacgcacccgcggccgtggtgtcgtggccacccgttgggtgccgcaaatgtccattctggctcatgcggccgttggcgcatttctgacccactgcggttggaacagcacgatcgaaggcctgatgtttggtcatccgctgattatgctgccgatcttcggcgatcagggtccgaacgcacgcctgatcgaagccaaaaatgcaggcctgcaagttgcgcgtaacgatggcgacggtagctttgaccgcgaaggtgtcgcagctgcgattcgtgctgtggcggttgaagaagaaagcagcaaagtcttccaggccaaagcgaaaaaactgcaagaaatcgtggctgatatggcgtgtcatgaacgctatattgacggctttatccagcaactgcgttcttacaaagatgacagtggctatagttcctcatacgccgcagctgcgggtatgcatgttgtcatttgcccgtggctggcgtttggtcacctgctgccgtgtctggatctggcacagcgcctggcatctcgcggtcaccgtgtttcgttcgtcagcaccccgcgcaatatcagtcgtctgccgccggttcgtccggcgctggcgccgctggttgcgttcgttgcactgccgctgccgcgtgtggaaggtctgccggatggtgccgaatcgaccaacgacgttccgcatgatcgtccggacatggtcgaactgcatcgtcgcgcctttgatggcctggccgcaccgtttagcgaatttctgggtacggcctgcgcagattgggtcattgtggacgtttttcaccactgggcggcggcggcggcgctggaacataaagtgccgtgtgcgatgatgctgctgggttccgcccacatgattgcttcaatcgcggatcgtcgcctggaacgtgccgaaaccgaaagtccggcggcggcaggccagggtcgtccggcggcggcaccgacctttgaagtggcacgtatgaaactgattcgcacgaaataa

SEQ ID NO: 25 - protein, 458 aa, Artificial Sequence
  Synthetic polypeptide
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 26 - DNA, 1377 bp, Artificial Sequence
  Synthetic polynucleotide
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 27 - protein, 384 aa, Acrostichum aureum;
MAPTPSSSYTPKNILITGAAGFIASHVANRLVRLYPDYKIVVLDKLDYCSNLKNLFPSLPSPNFKFVKGDISSADLVNYLLMTEGIDTIMHFAAQTHVDNSFGNSFEFTKNNVYGTHVLLEACKVSGQIRRFIHVSTDEVYGETEADAIVGNHEASQLLPTNPYSASKAGAEMLVMAYGRSYGLPFITTRGNNVYGPNQFPEKLIPKFILLALQGKPLPIHGDGSNVRSYLFCEDVAEAFELVLHKGEVGHVYNIGTHKERRVLDVAKDICRLFKLDAEKSIQFVDNRPFNDQRYFLDDKKLKGLGWNERTTWEEGLQKTMDWYMRHPDWWGDVSGALLPHPRMLAMGGIDKTADLTQLPEFANGLGTDKKMAEAQANGGSVQV

SEQ ID NO: 28 - DNA, 1155 bp, Acrostichum aureum;
atggcaccgaccccgagcagcagttataccccgaaaaatattctgattaccggcgccgccggttttattgcaagccatgtggccaatcgtctggttcgcctgtatccggattataaaattgtggttctggataaactggattattgcagcaatctgaaaaatctgtttccgagtctgccgagtccgaattttaaatttgttaaaggtgacatcagcagtgccgatctggttaattatctgctgatgaccgaaggtattgataccattatgcattttgcagcccagacccatgttgataatagctttggtaatagctttgagtttactaaaaacaacgtgtatggcacccatgtgctgctggaagcctgcaaagttagtggccagattcgccgctttattcatgtgagcaccgatgaagtgtatggcgaaaccgaagccgatgccattgtgggcaatcatgaagccagccagctgctgccgaccaatccgtatagtgccagtaaagccggcgccgaaatgctggttatggcctatggtcgcagttatggtctgccgtttattaccacccgtggtaataatgtgtatggcccgaatcagtttccggaaaaactgattccgaaattcattctgctggccctgcaaggtaaaccgctgccgattcatggtgacggcagcaatgttcgcagttatctgttttgtgaagatgtggccgaagcatttgaactggtgctgcataaaggcgaagtgggccatgtttataatattggtacccataaagagcgtcgcgttctggatgtggcaaaagatatttgtcgtctgtttaaactggatgcagaaaaaagcattcagtttgtggataatcgcccgtttaatgatcagcgttattttctggatgataaaaaactgaagggcctgggctggaatgaacgcaccacctgggaagaaggtctgcaaaaaaccatggattggtatatgcgtcatccggattggtggggtgacgtgagtggtgcactgctgccgcatccgcgtatgctggccatgggcggcattgataaaaccgcagatttgacccagctgccggaatttgccaatggcctgggtaccgataaaaagatggcagaagcacaggccaatggcggtagcgtgcaggtgtaa

SEQ ID NO: 29 - protein, 362 aa, Ettlia oleoabundans;
MVQNGVLNGLQEDTFTPRVILVTGGAGFIGSHVAIRLLKRYPESYKVVVYDKMDYCASLKNLAELQGNPHYKCIRGDIQAADLVQYVLKEEAVDTVLHFAAQTHVDNSFGNSLAFTINNTYGTHVLLEACRMYGGVRRFIYVSTDEVYGDTSVGALAGLPESSSLAPTNPYSAAKAGAELMTLAYLTSYKLPVIITRSNNVYGPHQFPEKLIPKFVLLASRGERLPVHGDGLATRSYLYVGDVAEAFDIILHKGEVGQIYNIGSQQERTVLDVAADMCALFRLPPASQVEHVRDRAFNDRRQACPAAAARGQSHGGCLSWGWRHDGAAGSAWHCWWHLTAPAAQPSKQALPDCTVLEQVFHL

SEQ ID NO: 30 - DNA, 1089 bp, Ettlia oleoabundans;
atggttcagaatggcgttctgaatggcctgcaagaagatacctttaccccgcgtgttattctggtgaccggtggtgccggttttattggtagccatgtggccattcgtctgctgaaacgttatccggaaagctataaagttgtggtttatgataagatggactattgtgccagcctgaaaaatctggccgaactgcaaggtaatccgcattataaatgtattcgcggcgatattcaggccgcagatttggttcagtatgtgctgaaagaagaagccgtggataccgtgctgcattttgccgcccagacccatgtggataatagctttggtaatagcctggcctttaccattaataatacctatggcacccatgttctgctggaagcctgccgtatgtatggtggtgtgcgtcgttttatctatgtgagtaccgatgaagtttatggtgacaccagcgttggtgccctggccggcctgcctgaaagcagtagtctggccccgaccaatccgtatagcgccgcaaaagccggtgccgaactgatgaccctggcctatctgaccagctataaactgccggttattattacccgcagcaataatgtttatggcccgcatcagtttccggaaaaactgattccgaaatttgttctgctggccagccgtggcgaacgcctgcctgtgcatggcgatggtctggcaacccgtagctatctgtatgtgggtgacgttgcagaagcatttgatattattctgcataaaggtgaagtgggtcagatatataatattggtagtcagcaggaacgtaccgttctggatgttgcagcagatatgtgcgcactgtttcgcctgccgccggccagccaggttgaacatgtgcgcgatcgtgcctttaatgatcgccgtcaggcctgcccggccgcagcagcaagaggtcagagccatggcggctgcctgagctggggctggcgtcatgatggcgccgcaggcagtgcatggcattgctggtggcatctgaccgccccggcagcacagccgagcaaacaggccctgccggattgtaccgttctggaacaggtttttcatctgtaa

SEQ ID NO: 31 - protein, 366 aa, Volvox carteri;
MASIDNGIGESEPYTPKNILITGGAGFIASHVVIRIATRYPEYKVVVLDKLDYCASVNNLSCLADKPNFRLIKGDIQSMDLISYILKTEEIDTVMHFAAQTHVDNSFGNSLAFTLNNTYGTHVLLEASRMAGTIRRFINVSTDEVYGETSLGKTTGLVESSHLDPTNPYSAAKAGAELIARAYITSYKMPVIITRGNNVYGPHQFPEKLIPKFTLLAARGKELPLHGDGSSVRSYLYVEDVAEAFDCVLHKGVTGETYNIGTDRERSVLEVARDIAKLFNLPEDKVVFVKDRAFNDRRYYIGSAKLAALGWQERTSWEEGLRKTVDWYLGLKNIENYWAGDIEMALRPHPIVVQNAITTSGAFLAS

SEQ ID NO: 32 - DNA, 1101 bp, Volvox carteri;
atggcaagtattgataacggtattggtgaaagtgaaccgtataccccgaaaaatattctgattaccggcggtgccggctttattgcaagccatgttgttattcgtattgccacccgttatccggaatataaagttgtggtgctggataaactggattattgcgccagtgtgaataatctgagctgcctggccgataaaccgaattttcgtctgattaagggcgatattcagagcatggatctgattagctatattctgaaaaccgaagaaatcgataccgtgatgcattttgcagcacagacccatgtggataatagttttggcaatagcctggcattcactctgaataatacctatggcacccatgttctgctggaagcaagccgcatggccggtaccattcgccgctttattaatgttagtaccgatgaagtttacggcgaaaccagtctgggcaaaaccaccggtctggttgaaagcagccatctggatccgaccaatccgtatagcgcagcaaaagcaggtgcagaactgattgcccgtgcatatattaccagttataaaatgccggttatcattacccgcggtaataatgtgtatggtccgcatcagtttccggaaaaactgattccgaaattcactctgctggcagcccgtggcaaagaactgccgctgcatggcgatggtagcagcgttcgcagctatctgtatgtggaagatgttgcagaagcctttgattgtgtgctgcataaaggtgttaccggtgaaacctataatattggcaccgatcgtgaacgcagtgtgctggaagttgcacgtgatattgcaaaactgtttaatctgccggaagataaagtggtttttgtgaaagatcgtgcattcaatgatcgtcgctattatattggtagtgcaaaactggcagcactgggctggcaggaacgcaccagttgggaagaaggcctgcgtaaaaccgttgattggtatctgggtctgaaaaatattgaaaattactgggccggcgatattgaaatggccctgcgcccgcatccgattgtggttcagaatgcaattaccaccagcggtgcctttctggccagctaa

SEQ ID NO: 33 - protein, 367 aa, Chlamydomonas reinhardtii;
MATSNGNGTPEVEPYEPKNILITGGAGFIASHVVIRITKNYPQYKVVVLDKLDYCASLKNLGSVANLPNFRFIKGDIQSMDLISYILKTEEIDTVMHFAAQTHVDNSFGNSLAFTLNNTYGTHVLLEAARMHGRIRRFINVSTDEVYGETSLGKTTGLVESSHLDPTNPYSAAKAGAELIARAYITSYKLPVIITRGNNVYGPHQFPEKLIPKFTLLANRGADLPIHGDGTSVRSYLYVEDVAEAFDCVLHKGVTGETYNIGTERERSVKEVAKDIAKFFNLPESKVVNVRDRAFNDRRYYIGSNKLGALGWTERTSWEDGLKKTIDWYINLPNRDEYWAGDVEMALKPHPVVNANAATVSGPFLAN

SEQ ID NO: 34 - DNA, 1104 bp, Chlamydomonas reinhardtii;
atggccaccagcaatggcaatggtaccccggaagtggaaccgtatgaaccgaaaaatattctgattaccggcggtgcaggttttattgccagccatgtggttattcgcattaccaaaaattatccgcagtataaagtggtggttctggataaactggattattgtgcaagtctgaaaaatctgggcagtgtggccaatctgccgaattttcgttttattaagggtgacattcagagcatggatctgattagttatattctgaaaaccgaagaaatcgataccgttatgcattttgcagcccagacccatgttgataatagctttggtaatagcctggcctttaccctgaataatacctatggtacccatgttctgctggaagccgcacgcatgcatggccgcattcgtcgttttattaatgtgagtaccgatgaagtgtatggcgaaaccagtctgggcaaaaccaccggcctggttgaaagtagccatctggatccgaccaatccgtatagcgccgcaaaagccggtgcagaactgattgcacgtgcctatattaccagctataaactgccggttattattacccgcggtaataatgtttatggcccgcatcagtttccggaaaaactgattccgaaattcactctgctggcaaatcgtggtgccgatctgccgattcatggcgatggcaccagcgtgcgtagttatctgtatgttgaagatgttgcagaagcctttgattgtgttctgcataaaggcgtgaccggcgaaacctataatattggcaccgaacgtgaacgcagtgttaaagaagtggccaaagatattgccaaatttttcaatctgccggaaagtaaagtggtgaatgttcgtgatcgtgcctttaatgatcgccgctattatattggcagtaataagctgggtgcactgggctggaccgaacgcaccagttgggaagatggtctgaaaaagactattgattggtatattaacctgccgaatcgtgatgaatattgggcaggtgacgttgaaatggcactgaaaccgcatccggtggttaatgcaaatgcagccaccgtgagcggtccgtttctggcaaattaa

SEQ ID NO: 35 - protein, 363 aa, Oophila amblystomatis;
MEGENGAEQCDYSPRCILVTGGAGFIASHVAIRLTKNYPQYKIVVLDKLDYCSSLKNLGAIKNSPNFKFVKGDIQSMDLIGFVIQSEEIDTVMHFAAQTHVDNSFGNSLAFTMNNIYGTHVLLEACRKAGTVRRFINVSTDEVYGETSLGKEKGLQESSHLDPTNPYSAAKAGAEMLCKAYLTSYKMPIIITRGNNVYGPHQFPEKMIPKFTILASRGESLPLHGDGSSIRSYLYVEDVAEAFDCVLHKGQVGDVYNIGTEQERTVVQVARDIAKHFGLASDKVVHVKDRAFNDRRYYIGSNKLAALGWSERTSWEEGLEKTIKWYLNTKIGEYWVGDVESALQPHPVVPVSATTLNSPHITL

SEQ ID NO: 36 - DNA, 1092 bp, Oophila amblystomatis;
atggaaggcgaaaatggtgcagaacagtgcgattatagcccgcgctgcattctggttaccggcggtgccggttttattgccagccatgtggccattcgtctgaccaaaaattatccgcagtataaaattgtggtgctggataaactggattattgtagcagcctgaaaaatctgggtgccattaagaatagtccgaattttaaattcgtgaagggcgatattcagagcatggatctgattggttttgtgattcagagcgaagaaattgataccgtgatgcattttgccgcccagacccatgttgataatagctttggcaatagcctggcctttaccatgaataatatctatggtacccatgttctgctggaagcctgccgtaaagcaggcaccgttcgtcgttttattaatgttagcaccgatgaagtgtatggcgaaaccagcctgggcaaagaaaaaggtctgcaagaaagtagtcatctggatccgaccaatccgtatagcgcagcaaaagccggcgccgaaatgctgtgtaaagcatatctgaccagttataaaatgccgattattattacccgcggcaataatgtgtatggcccgcatcagtttccggaaaaaatgattccgaaattcactattctggcaagccgcggcgaaagcctgccgctgcatggcgatggtagtagcattcgtagttatctgtatgttgaagatgtggcagaagcctttgattgtgtgctgcataaaggccaggtgggcgatgtttataatattggtaccgaacaggaacgcaccgtggtgcaggttgcacgtgatattgcaaaacattttggtctggcaagcgataaagttgttcatgttaaagatcgcgcattcaatgatcgccgctattatattggcagtaataagctggccgccctgggttggagtgaacgcaccagctgggaagaaggtctggaaaaaaccattaagtggtatctgaataccaaaattggtgaatattgggtgggtgacgttgaaagcgcactgcaaccgcatccggttgttccggtgagcgcaaccaccctgaatagtccgcatattaccctgtaa

SEQ ID NO: 37 - protein, 346 aa, Dunaliella primolecta;
MSGTEVPYKPRCILVTGGAGFIASHVVIRLVHLHPEYKVVVLDKMDYCASMNNLATCVGKPNFKCIKGDVQSMDLLAFLLNSEEIDTVMHFAAQTHVDNSFGNSLAFTMNNTYGTHVLLEACRMAGTIRRFINVSTDEVYGESSFGKELGLLEHSHLDPTNPYSAAKAGAEMLCKAYITSYKLPIIITRGNNVYGPHQFPEKLIPKFTLLASRGETLPVHGAGDSVRSYLYVEDVAEAFLCVLHQGVTGEVYNIGTDSERTVLQVAQDIAKRFNMGVDKIVNVKDRAFNDRRYYIGSSKLAELGWKERTSWEEGLKKTVDWYLKTNCNEYWLGDVEAALKPHPVVM

SEQ ID NO: 38 - DNA, 1041 bp, Dunaliella primolecta;
atgagtggtaccgaagtgccgtataaaccgcgttgcattctggttaccggtggtgccggctttattgccagtcatgttgtgattcgtctggtgcatctgcatccggaatataaagttgtggtgctggataaaatggattattgtgccagtatgaataacctggcaacctgcgttggcaaaccgaattttaaatgtattaagggtgacgttcagagcatggatctgctggcctttctgctgaatagcgaagaaattgataccgtgatgcattttgccgcccagacccatgttgataatagctttggtaatagcctggcctttaccatgaataatacctatggcacccatgttctgctggaagcctgtcgtatggcaggtaccattcgtcgttttattaatgttagcaccgatgaagtttacggtgaaagcagttttggtaaagaactgggtctgctggaacatagtcatctggatccgaccaatccgtatagcgccgcaaaagccggtgcagaaatgctgtgtaaagcatatattaccagttataagctgccgattattattacccgcggcaataatgtgtatggtccgcatcagtttccggaaaaactgattccgaaattcactctgctggcaagtcgtggcgaaaccctgccggtgcatggtgcaggtgacagtgtgcgtagctatctgtatgttgaagatgttgccgaagcctttctgtgcgtgctgcatcagggtgttaccggtgaagtttataatattggtaccgatagcgaacgtaccgtgctgcaagttgcccaggatattgcaaaacgctttaatatgggcgtggataaaattgtgaatgtgaaagatcgcgcattcaatgatcgtcgttattatattggcagtagcaaactggcagaactgggctggaaagaacgtaccagttgggaagaaggtctgaaaaagactgttgattggtatctgaaaaccaattgtaatgaatactggctgggcgatgttgaagcagccctgaaaccgcatccggttgttatgtaa

SEQ ID NO: 39 - protein, 360 aa, Ostreococcus lucimarinus;
MRILLTGGAGFIGSHVAERLASRHPEYTIVILDKLDYCSSLKNLERAKECANVRFVKGDVRSFDLLSYVLQSERIDTVMHFAAQSHVDNSFGNSYEFTKNNIEGTHALLEACVRAQKTEIRRFLHVSTDEVYGENLMDSNTEHASLLTPTNPYAATKAGAEMLVMAYGRSYGLPYIITRGNNVYGPNQYPEKAIPKFSILAKRGEKISIHGDGDATRSYMHVDDASSAFDVILHRGTTAQIYNIGSREERTILSVARDVCKLLDRDPETTIEHVSDRAFNDRRYFIDCSKLLALGWRQEKSWDVGLAETVRWYSNNDLSAYWGEFSPALRPHPSASADGRRRSLEFDFTNELDDCTTLAL

SEQ ID NO: 40 - DNA, 1104 bp, Ostreococcus lucimarinus;
atgcgcattctgctgaccggtggcgcaggttttattggtagtcatgttgccgaacgcctggccagtcgtcatccggaatataccattgttattctggataaactggattattgcagcagcctgaaaaatctggaacgtgccaaagaatgcgccaatgtgcgctttgtgaaaggtgacgttcgtagttttgatctgctgagctatgttctgcaaagtgaacgcattgataccgtgatgcattttgcagcacagagccatgtggataatagctttggtaatagttatgagtttactaagaacaacatcgaaggcacccatgcactgctggaagcatgtgttcgtgcacagaaaaccgaaattcgccgctttctgcatgtgagtaccgatgaagtttatggtgaaaatctgatggatagcaataccgaacatgcaagtctgctgaccccgaccaatccgtatgcagcaaccaaagcaggtgccgaaatgctggttatggcatacggtcgcagttatggtctgccgtatattattacccgcggcaataatgtgtatggcccgaatcagtatccggaaaaagccattccgaaattttctattctggcaaaacgtggcgaaaaaattagcattcatggcgatggcgatgcaacccgtagctatatgcatgtggatgatgccagtagtgcctttgatgtgattctgcatcgtggtaccaccgcccagatatataatattggtagccgtgaagaacgtaccattctgagtgtggcacgtgatgtttgcaaactgctggatcgcgatccggaaaccaccattgaacatgttagcgatcgtgcctttaatgatcgccgttattttattgattgcagcaaactgctggccctgggctggcgccaggaaaaaagttgggatgttggtctggcagaaaccgttcgctggtatagcaataatgatctgagcgcctattggggcgaattttctccggcactgcgtccgcatccgagtgcaagcgccgatggtcgtcgtcgtagtctggaatttgattttaccaatgaactggatgattgcaccaccctggcactgtaaccaaacgtcttcagagagtaa

SEQ ID NO: 41 - protein, 356 aa, Nannochloropsis oceanica;
MSNGCAPVTAETDYTPKNILITGGAGFIASHVVLLLVKKFPKYKIVNLDRLDYCSCLENLDEIKYYKNYKFVKGNICSSDLVNYVLEEEEIDTIMHFAAQTHVDNSFGNSFSFTQNNILGTHVLLESAKVHGIKRFIHVSTDEVYGEGAADQEPMFEDQVLEPTNPYAATKAGAEFIAKSYSRSFNLPLIITRGNNVYGPHQYPEKLIPKFVNLLMRDRPVTLHGNGLNTRNFLFVEDVARAFEVILHRGVTGKIYNIGGTNEKANIEVAKDLIRLMGYEQAEEKMLNFVEDRAFNDLRYTVNSEALKQLGWEELVSWEDGLNKTVEWYKQYTGRYGNIDCALVAHPRSGALHEFP

SEQ ID NO: 42 - DNA, 1071 bp, Nannochloropsis oceanica;
atgagtaacggttgtgcaccggttaccgcagaaaccgattataccccgaaaaatattctgattaccggcggtgcaggttttattgcaagccatgttgtgctgctgctggtgaaaaaatttccgaaatataaaatcgtgaacctggatcgcctggattattgtagttgcctggaaaatctggatgaaattaagtattacaagaactacaagttcgtgaaaggtaatatttgcagcagcgatctggttaattatgttctggaagaagaagaaatcgataccattatgcattttgccgcacagacccatgtggataatagttttggtaatagtttcagcttcacccagaataatattctgggcacccatgtgctgctggaaagtgcaaaagttcatggcattaagcgttttattcatgtgagcaccgatgaagtttatggtgaaggtgcagccgatcaggaaccgatgtttgaagatcaggtgctggaaccgaccaatccgtatgcagccaccaaagcaggtgcagagtttattgcaaaaagctatagtcgcagctttaatctgccgctgattattacccgtggcaataatgtttatggtccgcatcagtatccggaaaaactgattccgaaatttgttaatctgctgatgcgcgatcgcccggttaccctgcatggtaatggcctgaatacccgtaattttctgtttgtggaagatgtggcccgtgcatttgaagtgattctgcatcgtggtgttaccggtaaaatctataatattggcggtaccaatgaaaaagcaaatattgaagttgcaaaggatctgattcgcctgatgggttatgaacaggccgaagaaaaaatgctgaattttgttgaagatcgtgcttttaatgacctgcgttataccgtgaatagtgaagccctgaaacagctgggctgggaagaactggtgagctgggaagatggcctgaataagaccgtggaatggtataaacagtataccggccgttatggcaatattgattgtgccctggttgcacatccgcgcagtggcgccctgcatgaatttccgtaa

SEQ ID NO: 43 - protein, 378 aa, Ulva lactuca;
MATNGETSAAETRGNNYGLARVMTNGEFVYEDKFVPKSILLTGGAGFIGSHVAILLAKKYPDYKIVVLDKLDYCATLNNLKEISSLPNFKFVRGCIQSFDLVAHVLETEEVDTVMHFAAQTHVDNSFGNSLEFTMNNTYGTHVLLEAARKHGKIRRFINVSTDEVYGESSLGKEQGCDETSTLEPTNPYSAAKAGAEMMVRSYMTSYKLPCIITRGNNVYGPHQFPEKLIPKMTLLANRGQPLPVHGNGQAVRSYLHVRDVARAFDTVLHKGVLGEVYNIGTQKERSVVDVVSAIAEYMKVDTAKIHHVEDRAFNDQRYYICDKKLLALGWKEEETWENGLGETVDWYLKNGTSDYWENGNMDAALVAHPTLAASVQK

SEQ ID NO: 44 - DNA, 1137 bp, Ulva lactuca;
atggccaccaatggtgaaaccagtgccgccgaaacccgtggtaataattatggcctggcccgtgttatgaccaatggtgagtttgtttatgaagataaattcgttccgaagagtattctgctgaccggcggtgcaggctttattggcagtcatgttgccattctgctggcaaaaaagtatccggattataaaattgtggtgctggataaactggattattgtgcaaccctgaataatctgaaagaaattagcagcctgccgaattttaaatttgtgcgtggctgtattcagagttttgatctggttgcccatgttctggaaaccgaagaagttgataccgttatgcattttgcagcccagacccatgtggataatagctttggcaatagtctggagtttactatgaataatacctatggcacccatgttctgctggaagcagcccgcaaacatggcaaaattcgtcgttttattaacgttagtaccgatgaagtttacggcgaaagcagcctgggtaaagaacagggttgtgatgaaaccagcaccctggaaccgaccaatccgtatagtgccgccaaagcaggcgcagaaatgatggtgcgcagctatatgaccagttataaactgccgtgtattattacccgtggcaataatgtgtatggtccgcatcagtttccggaaaaactgattccgaaaatgaccctgctggcaaatcgtggtcagccgctgccggttcatggtaatggtcaggccgtgcgtagctatctgcatgtgcgtgatgtggcccgtgcctttgataccgtgctgcataaaggtgtgctgggtgaagtttataatattggtacccagaaagaacgcagtgtggtggatgttgttagtgcaattgcagaatatatgaaagtggataccgcaaaaattcatcatgtggaagatcgtgcctttaatgatcagcgctattatatttgcgataaaaaactgctggcactgggctggaaagaagaagaaacctgggaaaatggcctgggcgaaaccgttgattggtatctgaaaaatggtaccagcgattattgggaaaatggtaatatggatgcagccctggtggcccatccgaccctggcagcaagcgttcagaaataa

SEQ ID NO: 45 - protein, 359 aa, Golenkinia longispicula;
MNGLGTFEPRNILLTGGAGFIGSHVAIRLLKKYPQYKVVILDCLDYCASLSNLSSVRKLPNFKFIKGDIQSADLVRLVLQQEEIDTVMHFAAQTHVDNSFGNSLAFTINNTYGTHVLLECCREYGQIQRFINVSTDEVYGESSLGRKEGLDESSALEPTNPYAAAKAGAEMMAKAYMTSYKLPVIITRGNNVYGPHQFPEKLIPKFTLLAHKGRDLPVHGDGGAVRSYLYVEDVAAAFDTVLHYGKLGEVYNIGSKVERSVLSVAQDIASYFGAPLNKIVYVRDRAFNDRRYFICDKKLAALGWKESVSWEEGLRRTIDWYVMKGSKQEYWDNGDLEAALQPHPTSQPRGMTAQSPYQA

SEQ ID NO: 46 - DNA, 1080 bp, Golenkinia longispicula;
atgaacggtctgggtacctttgaaccgcgcaatattctgctgaccggcggcgccggttttattggtagtcatgttgccattcgtctgctgaaaaaatatccgcagtataaagtggttattctggattgtctggattattgtgccagcctgagtaatctgagcagtgttcgtaaactgccgaattttaaattcattaagggcgatattcagagcgccgatctggttcgtctggttctgcaacaggaagaaattgataccgtgatgcattttgcagcccagacccatgttgataatagttttggtaatagcctggcctttaccattaataatacctatggtacccatgtgctgctggaatgctgtcgcgaatatggccagattcagcgttttattaatgtgagtaccgatgaagtttacggcgaaagtagcctgggccgcaaagaaggcctggatgaaagtagtgcactggaaccgaccaatccgtatgcagcagccaaagcaggtgcagaaatgatggcaaaagcctatatgaccagttataaactgccggttattattacccgtggcaataatgtgtatggcccgcatcagtttccggaaaaactgattccgaaattcactctgctggcacataaaggtcgcgatctgccggtgcatggcgatggtggtgccgttcgcagttatctgtatgtggaagatgtggcagcagcctttgataccgttctgcattatggcaaactgggtgaagtgtataatattggcagtaaagtggaacgcagcgttctgagcgtggcacaggatattgcaagttattttggcgcaccgctgaataagattgtttatgttcgtgatcgtgcctttaatgatcgccgctattttatttgtgataaaaaactggccgccctgggctggaaagaaagcgtgagttgggaagaaggtctgcgtcgcaccattgattggtatgtgatgaaaggcagtaaacaggaatattgggataatggcgatctggaagccgcactgcaaccgcatccgaccagccagccgcgcggtatgaccgctcagagtccgtatcaggcctaa

SEQ ID NO: 47 - protein, 359 aa, Tetraselmis subcordiformis;
MTGEAEVGSNGHRHAEFQPKNILVTGGAGFIGSHVVLRLLRNYPAYKVVVLDKLDYCASLRNLREAEGSKQYKFIKGDIQSADLISFILQTEEIDTVMHFAAQTHVDNSFGNSLTFTMNNTYGTHVLLESCRVYGGIKRFINVSTDEVYGESSLGSQTGLDETSKMEPTNPYSAAKAGAEMLARAYITSYKMPIIITRGNNVYGPHQFPEKMIPKFTLLASRGANLPVHGDGNALRSYLYVEDVAHAFDVVLHAGVTGETYNIGTQKERSVIEVAKAIANIFKMPEDRVVHVKDRAFNDRRYYICDDKLNALGWAESTPWEEGLKKTVDWYLYNGFAGYWAEAEVELALQAHPTLRQSV

SEQ ID NO: 48 - DNA, 1080 bp, Tetraselmis subcordiformis;
atgaccggtgaagccgaagtgggcagcaatggtcatcgccatgcagaatttcagccgaaaaatattctggttaccggcggcgcaggctttattggtagtcatgttgtgctgcgtctgctgcgtaattatccggcctataaagttgttgtgctggataaactggattattgtgcaagcctgcgcaatctgcgtgaagcagaaggtagtaaacagtataaattcattaagggtgacatccagagtgcagatttgattagctttattctgcaaaccgaagaaattgataccgtgatgcattttgcagcacagacccatgttgataatagctttggcaatagtctgacctttaccatgaataatacctatggcacccatgttctgctggaaagctgccgtgtgtatggcggcattaagcgctttattaatgttagcaccgatgaagtttacggcgaaagcagcctgggcagccagaccggcctggatgaaaccagcaaaatggaaccgaccaatccgtatagcgccgccaaagcaggtgccgaaatgctggcacgtgcatatattaccagttataaaatgccgattatcatcacccgcggcaataatgtttatggtccgcatcagtttccggaaaaaatgattccgaaattcactctgctggccagccgtggtgcaaatctgccggttcatggtgacggtaatgcactgcgtagctatctgtatgttgaagatgttgcccatgcatttgatgttgttctgcatgccggcgtgaccggtgaaacctataatattggcacccagaaagaacgtagcgttattgaagttgcaaaagcaattgcaaatatctttaagatgccggaagatcgtgtggtgcatgtgaaagatcgcgcctttaatgatcgtcgttattatatttgtgacgataaactgaacgcactgggctgggccgaaagtaccccgtgggaagaaggcctgaaaaagactgttgattggtatctgtataacggttttgcaggctattgggcagaagccgaagttgaactggccctgcaagcacatccgaccctgcgtcagagcgtttaa

SEQ ID NO: 49 - protein, 300 aa, Physcomitrella patens;
MVATVNGGQSAGLKFLIYGKTGWIGGLLGKLCTEQGIAYEYGKGRLENRSSIEQDISTVKPTHVFNAAGVTGRPNVDWCESHKIETIRANVVGTLTLADVCKQNDLVLVNYATGCIFEYDDAHPLGSGIGFKEEESANFRGSYYSKTKAMVEELLREFDNVCTLRVRMPITGDLSNPRNFITKITRYEKVVDIPNSMTILDELLPISIEMAKRNLTGIWNFTNPGVVSHNEILEMYKEYVDPSFTYKNFTLEEQAKVIVAARSNNELDASKLSKEFPEMLPIKESLIKYVFEPNKKTNKP

SEQ ID NO: 50 - DNA, 924 bp, Physcomitrella patens;
atggtggcaaccgttaatggcggccagagtgccggtctgaaatttctgatctatggtaaaaccggctggattggtggtctgctgggcaaactgtgtaccgaacagggtattgcatacgaatatggcaaaggccgcctggaaaatcgcagcagcattgaacaggatattagcaccgtgaaaccgacccatgtgtttaatgcagcaggtgttaccggccgtccgaatgttgattggtgtgaaagtcataaaatcgaaaccattcgtgccaatgtggttggcaccctgaccctggcagatgtttgcaaacagaatgatctggtgctggttaattatgccaccggttgcatttttgaatatgatgatgcccatccgctgggtagtggtattggttttaaagaagaagaaagtgcaaactttcgtggtagctattatagtaaaaccaaagccatggtggaagaactgctgcgtgaatttgataatgtttgtaccctgcgtgtgcgcatgccgattaccggtgacctgagtaatccgcgtaattttattaccaaaatcacccgttatgagaaagtggttgatattccgaatagtatgaccattctggatgaactgctgccgattagcattgaaatggcaaaacgtaatctgaccggtatttggaattttaccaatccgggtgtggttagtcataatgaaattctggaaatgtacaaggaatacgtggatccgagttttacctataaaaattttaccctggaggaacaggccaaagttattgtggcagcacgtagcaataatgaactggatgccagcaaactgagcaaagaatttccggaaatgctgccgattaaggaaagtctgattaagtatgttttcgaaccgaataagaaaactaataagccgtaaccaaacgtcttcagagagtaa

SEQ ID NO: 51 - protein, 292 aa, Pyricularia oryzae;
MTNNRFLIWGGEGWVAGHLASILKSQGKDVYTTTVRMENREGVLAELEKVKPTHVLNCAGCTGRPNVDWCEDNKEATMRSNVIGTLNLTDACFQKGIHCTVFATGCIYQYDDAHPWDGPGFLETDKANFAGSFYSETKAHVEEVMKYYNNCLILRLRMPVSDDLHPRNFVTKIAKYDRVVDIPNSNTILHDLLPLSLAMAEHKDTGVYNFTNPGAISHNEVLTLFRDIVRPSFKWQNFSLEEQAKVIKAGRSNCKLDTTKLTEKAKEYGIEVPEIHEAYRQCFERMKKAGVQ

SEQ ID NO: 52 - DNA, 900 bp, Pyricularia oryzae;
atgaccaataaccgttttctgatttggggtggcgaaggctgggtggccggccatctggctagcattctgaaaagccagggcaaagatgtttataccaccaccgtgcgtatggaaaatcgtgaaggtgttctggccgaactggaaaaagtgaaaccgacccatgttctgaattgtgcaggctgtaccggtcgtccgaatgttgattggtgcgaagataataaggaagccaccatgcgtagtaatgttattggtaccctgaatctgaccgatgcatgctttcagaaaggtattcattgcaccgtgtttgcaaccggctgtatctatcagtatgatgatgcacatccgtgggatggcccgggttttctggaaaccgataaagcaaattttgccggtagcttttatagtgaaaccaaagcccatgtggaagaagtgatgaaatattataacaactgcctgattctgcgcctgcgtatgccggtgagtgatgatctgcatccgcgtaattttgttaccaaaattgcaaaatacgaccgtgtggttgatattccgaatagtaataccattctgcatgatctgctgccgctgagtctggccatggcagaacataaagataccggtgtttataatttcaccaatccgggtgccattagtcataatgaagtgctgaccctgtttcgcgatattgtgcgtccgagttttaaatggcagaattttagtctggaagaacaggcaaaagttattaaggcaggccgtagcaattgtaaactggataccaccaaactgaccgaaaaagccaaagaatatggtattgaagtgccggaaattcatgaagcctatcgccagtgttttgaacgtatgaaaaaagcaggtgttcagtaaccaaacgtcttcagagagtaa

SEQ ID NO: 53 - protein, 300 aa, Nannochloropsis oceanica;
MSEEKYLIFGKNGWIGGKLIDLLKQQGKTVVLGQSRLENREALFAELDDVKPTHVLDAAGVTGRPNIDWCETHQVETIRTNVIGTLNLAEGCHLKGIHMTLYATGCIFEYDEKHPIGGPGFTEEDSPNFFGSFYSKTKAYMEDMLKSYKNVCILRVRMPISDDLNPRNFVTKIVSYDRVVDVPNSMTVLTDLLPISLIMSQRKLTGIYNFTNPGAISHNQILTLYKKHVDPSYTWQNFTIEEQNKILAAKRSNNELDTTKFCAALPDIQIPDIHAACEGVRTPPSLPSSLPVSLLSLGAE

SEQ ID NO: 54 - DNA, 903 bp, Nannochloropsis oceanica;
atgagtgaagaaaagtacctgattttcggtaaaaatggctggattggtggcaaactgattgatctgctgaaacagcagggcaaaaccgtggtgctgggccagagtcgtctggaaaatcgcgaagcactgtttgccgaactggatgatgttaaaccgacccatgtgctggatgccgccggcgtgaccggtcgtcctaatattgattggtgcgaaacccatcaggttgaaaccattcgtaccaatgttattggcaccctgaatctggcagaaggttgccatctgaaaggtattcacatgaccctgtatgcaaccggttgtatttttgaatatgatgaaaagcacccgattggtggtccgggctttaccgaagaagatagtccgaatttctttggcagtttttatagtaaaaccaaggcctatatggaagatatgctgaaaagttataagaacgtttgtatcctgcgtgttcgcatgccgattagtgatgatctgaatccgcgcaattttgttaccaaaattgttagttacgaccgtgtggttgatgtgccgaatagcatgaccgtgctgaccgatctgctgccgattagcctgattatgagtcagcgcaaactgaccggcatctataattttaccaatccgggcgcaattagccataatcagattctgaccctgtataaaaaacatgttgatccgagttatacctggcagaattttaccattgaagaacagaataagatcctggcagccaaacgtagcaataatgaactggataccaccaaattttgtgcagccctgccggatattcagattccggatattcatgccgcctgcgaaggtgttcgcaccccgcctagcctgccgagtagcctgccggttagtctgctgagtctgggtgccgaataa

SEQ ID NO: 55 - protein, 307 aa, Ulva lactuca;
MAEEPKFLIFGKSGWIGGLVGEELERQGAKYEYGTARLENREAILADIERVKPTHVLNCAGITGRPNVDWCEDHKIECIRGNVLGTINLADVTNEKGIHMVYYGTGCIFHYDEEFKVNTGKGFKEGDKPNFTGSYYSHCKAMTENLLQAFPNVLTLRVRMPIVADLTYPRNFITKIIKYFKVVNIPNSMTVLPELIPLSIEMSKRKLTGIMNYTNPGAISHNEILELYKEYIDPDFTWENFDIEEQAKVIVAPRSNNLLDTDRMKGEFPELLGIKESLIKYVFEPNAKKKDEVKAAVDAMREEFRKA

SEQ ID NO: 56 - DNA, 924 bp, Ulva lactuca;
atggccgaagaaccgaaatttctgatttttggcaaaagcggctggattggcggtctggtgggcgaagaactggaacgccagggcgcaaaatatgaatatggtaccgcccgtctggaaaatcgtgaagccattctggccgatattgaacgtgtgaaaccgacccatgttctgaattgcgcaggtattaccggtcgcccgaatgtggattggtgtgaagatcataaaattgaatgtatccgtggcaatgtgctgggcaccattaatctggccgatgttaccaatgaaaaaggtattcacatggtttattacggtaccggctgcatttttcattatgatgaagagtttaaagtgaacaccggtaaaggttttaaagaaggcgataaaccgaattttaccggcagctattatagccattgcaaagccatgaccgaaaatctgctgcaagcatttccgaatgttctgaccctgcgtgttcgtatgccgattgttgcagatttgacctatccgcgcaattttattaccaaaattatcaaatacttcaaggtggtgaacattccgaatagcatgaccgttctgccggaactgattccgctgagcattgaaatgagtaaacgtaaactgaccggtattatgaattataccaatccgggcgccattagtcataatgaaattctggaactgtacaaagaatacattgatccggattttacctgggaaaattttgatattgaggaacaggcaaaagttattgtggcaccgcgtagcaataatctgctggataccgatcgtatgaaaggcgaatttccggaactgctgggcattaaggaaagtctgattaagtatgttttcgaaccgaatgcaaaaaagaaagatgaagttaaagccgccgttgatgcaatgcgtgaagaatttcgcaaagcctaa

SEQ ID NO: 57 - protein, 282 aa, Tetraselmis cordiformis;
MGELLEKQGIPFEFGTARLEDRTAIMADIERVKPTRILNAAGVTGRPNVDWCEENKQTCVRGNVIGTLNLADVCDKTGIHMIYYGTGCIFHYDDEFPENSGKGFKESDKPNFTGSYYSHCKAMTENLLQAFNNVLTLRVRMPIVQDVLYPRNFITKIIKYQKVINIPNSMTVLPELLPLSLEMSKRKLTGIMNFTNPGAISHNEILQLYKEFIDPEFSWQNFTVEEQAKVIVAPRSNNLLDTARIEGEFPEILGIKESLIKYVFEPLAQNKEVVCADVRKMR

SEQ ID NO: 58 - DNA, 849 bp, Tetraselmis cordiformis;
atgggtgaactgctggaaaaacagggcattccgtttgaatttggcaccgcacgcctggaagatcgtaccgccattatggcagatattgaacgtgtgaaaccgacccgtattctgaatgcagccggtgttaccggccgcccgaatgtggattggtgcgaagaaaataagcagacctgtgtgcgtggtaatgtgattggcaccctgaatctggcagatgtttgtgataaaaccggcattcacatgatctattatggcaccggttgcatttttcattatgatgatgaatttccggagaatagtggcaaaggttttaaagaaagtgataaaccgaactttaccggcagttattatagtcattgcaaagcaatgaccgaaaatctgctgcaagcattcaataatgtgctgaccctgcgtgttcgtatgccgattgttcaggatgtgctgtatccgcgtaattttattaccaaaattatcaagtaccagaaggttattaacatcccgaatagtatgaccgttctgccggaactgctgccgctgagtctggaaatgagcaaacgcaaactgaccggcattatgaattttaccaatccgggtgcaattagtcataatgaaattctgcaactgtacaaagagtttattgatccggaattttcatggcagaattttaccgttgaagaacaggccaaagtgattgtggccccgcgcagcaataatctgctggataccgcacgcattgaaggcgaatttccggaaattctgggtattaaggaaagtctgattaagtatgttttcgaaccgctggcacagaataaggaagtggtgtgcgccgatgtgcgcaaaatgcgttaa

SEQ ID NO: 59 - protein, 296 aa, Tetraselmis subcordiformis;
MTRSVEGNGAVKFLVYGRNGWIGSLLGELLKQQGADYEYGTARLEDRAAILADIERVKPTRVLNAAGITGRPNVDWCEDNRQTCIRGNVIGTLNLVDVCEQQGLHVTYFGTGCIFHYDDDFPEGSGKGFKESDTPNFTGSFYSHCKAMTENLLGAYSNVLTLRVRMPIVQDILYPRNFITKIIKYRKVIDIPNSMTVLPELLPYSLEMSRRALTGVMNFTNPGAISHNEILQLYKEYIDPDFTWENFTVEEQAKVIVAPRSNNLLDTERMKAEFPELLDIRQSLITHVFEPLSRNK

SEQ ID NO: 60 - DNA, 891 bp, Tetraselmis subcordiformis;
atgacccgcagcgttgaaggtaatggtgcagttaaatttctggtgtatggtcgcaatggttggattggtagcctgctgggcgaactgctgaaacagcagggcgcagattatgaatatggcaccgcccgtctggaagatcgcgcagcaattctggccgatattgaacgtgttaaaccgacccgtgtgctgaatgcagccggcattaccggccgtccgaatgttgattggtgtgaagataatcgccagacctgtattcgcggtaatgttattggtaccctgaatctggttgatgtgtgtgaacagcagggtctgcatgtgacctattttggtaccggttgtatttttcattacgatgatgatttcccggaaggtagtggcaaaggttttaaagaaagtgataccccgaattttaccggtagcttttatagtcattgtaaagccatgaccgaaaatctgctgggcgcctatagcaatgttctgaccctgcgtgtgcgtatgccgattgttcaggatattctgtatccgcgtaattttattaccaaaattatcaagtaccgtaaggttattgacattccgaatagcatgaccgttctgccggaactgctgccgtatagcctggaaatgagccgtcgtgccctgaccggcgttatgaattttaccaatccgggcgccattagccataatgaaattctgcaactgtataaagagtacattgatccggattttacctgggaaaattttaccgttgaagaacaggcaaaagtgattgttgccccgcgcagtaataatctgctggataccgaacgtatgaaagcagaatttccggaactgttagatattcgtcagagcctgattacccatgtgtttgaaccgctgagccgcaataagtaa

SEQ ID NO: 61 - protein, 313 aa, Chlorella sorokiniana;
MTVAQNVEAVAAEPTFLIYGRNGWIGGLVGEMLKKQGAKFEYGTARLEDRAAILADIERVKPTHVLNAAGVTGRPNVDWCETHKVETIRANVIGCLNLADVCLQNGIHMTYYGTGCIFHYDDGKFKQGNGVGFQESDTPNFTGSYYSHCKAMVENLLKEFPNVLTLRVRMPIVGDLVYPRNFITKIIKYDKVVDIPNSMTVLPELLPMSIEMAKRKLTGIMNFTNPGAISHNEILELYKQYVDPEFTWSNFTLEEQAKVIVAPRSNNLMASDRIKSEFPEILSIKESLIKYVFEPAAANREETLAAVREMRGR

SEQ ID NO: 62 - DNA, 942 bp, Chlorella sorokiniana;
atgaccgtggcacagaatgttgaagccgttgccgccgaaccgacctttctgatctatggtcgcaatggttggattggcggtctggtgggcgaaatgctgaaaaaacagggcgccaaatttgaatatggcaccgcccgtctggaagatcgtgcagccattctggccgatattgaacgtgttaaaccgacccatgtgctgaatgccgccggcgtgaccggccgtcctaatgtggattggtgcgaaacccataaagttgaaaccattcgtgcaaatgtgattggctgcctgaatctggccgatgtgtgcctgcaaaatggtattcacatgacctattatggcaccggttgcatttttcattatgatgatggtaaattcaagcagggcaatggtgtgggttttcaggaaagcgataccccgaattttaccggcagttattatagccattgtaaagcaatggtggaaaatctgctgaaagaatttccgaatgttctgaccctgcgtgtgcgcatgccgattgttggcgatctggtgtatccgcgtaattttattaccaaaattatcaagtacgacaaggtggttgatattccgaatagtatgaccgttctgccggaactgctgccgatgagcattgaaatggccaaacgcaaactgaccggcattatgaattttaccaatccgggtgcaattagccataatgaaattctggaactgtataaacagtacgttgatccggagtttacttggagcaattttaccctggaagaacaggcaaaagttattgtggccccgcgtagcaataatctgatggccagtgatcgtattaagagcgaatttccggaaattctgagcattaaggaaagtctgattaagtatgttttcgaaccggccgcagccaatcgcgaagaaaccctggccgcagttcgtgaaatgcgcggccgttaa

SEQ ID NO: 63 - protein, 303 aa, Chlamydomonas moewusii;
MAEKEPVFLVFGKSGWIGGLLGELLKEQGAKYEFASCRLEDRAAIISEIDRVKPTHVLNAAGLTGRPNVDWCETHKVETIRSNVIGCLNLADVCNQREIHMTYYGTGCIFHYDDTHPVGGEGFKEEDKPNFTGSYYSHTKAIVENLLKEFPNVLTLRVRMPIVEDLLYPRNFITKIIKYDKVVDIPNSMTVLPELLPYSIEMARRKLTGIMNFTNPGTVSHNEVLQLYKDYIDPEFTWSNFTIEEQAKVIVAPRSNNLLDTKRIESEFPMILPIKESLKKYVFEPSAEKKAELRAAVKEMRGR

SEQ ID NO: 64 - DNA, 912 bp, Chlamydomonas moewusii;
atggcagaaaaagaaccggtgtttctggtttttggtaaaagcggctggattggcggtctgctgggcgaactgctgaaagaacagggtgccaaatatgaatttgccagttgccgcctggaagatcgtgccgccattattagtgaaattgatcgtgttaaaccgacccatgttctgaatgccgccggcctgaccggccgtcctaatgttgattggtgcgaaacccataaagttgaaaccattcgtagtaatgtgattggctgcctgaatctggccgatgtgtgtaatcagcgtgaaattcacatgacctattatggtaccggctgcatttttcattatgatgatacccatccggtgggcggtgaaggttttaaagaagaagataaaccgaatttcaccggtagctattatagtcataccaaagcaattgtggaaaatctgctgaaagagtttccgaatgtgctgaccctgcgtgtgcgtatgccgattgtggaagatttgctgtatccgcgtaattttattaccaaaattatcaagtacgacaaggttgttgatattccgaatagtatgaccgttctgccggaactgctgccgtatagcattgaaatggcccgccgtaaactgaccggcattatgaattttaccaatccgggtaccgtgagccataatgaagtgctgcaactgtataaagattatattgatccggagtttacttggagtaattttaccattgaagagcaggccaaagttattgttgcaccgcgtagtaataatctgctggataccaaacgcattgaaagtgaatttccgatgattctgccgattaaggaaagcctgaaaaaatatgttttcgaaccgagcgccgaaaagaaagccgaactgcgcgccgccgttaaagaaatgcgtggtcgttaa

SEQ ID NO: 65 - protein, 277 aa, Golenkinia longispicula;
MGAKYSYATARLEDRTTIVDNIERVKPTHVLHAAGLTGRPNVDWCETHKIETIRSNVIGCLNLADVCHQRNIHMTYYGTGCIFHYDADFPMGSGKGFTEEDKPNFTGSYYSYTKAMVESLLKEYPNVLTLRVRMPIVADLTYPRNFIAKIIKYDKVVDIPNSMTVLPELLPMSIEMAKRNLTGVMNFTNPGAISHNEILQLYKEYVDEEFSWDNFTLEEQSKILAAPRSNNLMDTNKIQSEFPEILGIRESLIKYVFEPAAKRKEEVKAAVREMRGR

SEQ ID NO: 66 - DNA, 834 bp, Golenkinia longispicula;
atgggcgcaaaatatagctatgccaccgcccgcctggaagatcgtaccaccattgttgataatattgaacgtgtgaaaccgacccatgttctgcatgcagccggtctgaccggccgtccgaatgtggattggtgcgaaacccataaaattgaaaccattcgcagcaatgttattggttgtctgaatctggcagatgtgtgtcatcagcgtaatattcacatgacctattatggcaccggctgcatttttcattatgatgcagattttccgatgggtagtggtaaaggttttaccgaagaagataaaccgaattttaccggtagctattatagctataccaaagcaatggtggaaagtctgctgaaagaatatccgaatgtgctgaccctgcgcgttcgtatgccgattgtggcagatttgacctatccgcgcaattttattgccaaaattattaagtacgacaaggttgttgacattccgaatagtatgaccgtgctgccggaactgctgccgatgagtattgaaatggcaaaacgcaatctgaccggtgttatgaattttaccaatccgggtgccattagccataatgaaattctgcaactgtataaagagtacgttgatgaagaattttcctgggataattttaccctggaagaacagagtaaaattctggccgcaccgcgcagtaataatctgatggataccaataagatccagagcgaatttccggaaattctgggcattcgtgaaagcctgattaagtatgtttttgaaccggccgcaaaacgtaaagaagaagttaaagccgccgttcgtgaaatgcgtggtcgttaa

SEQ ID NO: 67 - protein, 310 aa, Chlamydomonas reinhardtii;
MAGDKTNGAAEPVFLLFGKSGWIGGLLQEELKKQGAKFHLADARMEDRSAVVADIEKYKPTHVLNAAGLTGRPNVDWCETHKLETIRANVIGCLTLADVCNQRGIHMTYYGTGCIFHYDDDFPVNSGKGFKESDKPNFTGSYYSHTKAIVEDLIKQYDNVLTLRVRMPIIADLTYPRNFITKIIKYDKVINIPNSMTVLPELLPMSLEMAKRGLTGIMNFTNPGAVSHNEILEMYKEYIDPEFTWSNFSVEEQAKVIVAPRSNNLLDTARIEGEFPELLPIKESLRKYVFEPNAAKKDEVYKAVKEMRGR

SEQ ID NO: 68 - DNA, 933 bp, Chlamydomonas reinhardtii;
atggcaggtgacaaaaccaatggcgcagcagaaccggtttttctgctgtttggtaaaagtggttggattggtggtctgctgcaagaagaactgaaaaaacagggcgcaaaatttcatctggccgatgcccgcatggaagatcgtagtgcagttgtggccgatattgaaaaatataaaccgacccatgttctgaatgcagccggcctgaccggtcgtccgaatgttgattggtgcgaaacccataaactggaaaccattcgcgccaatgttattggttgtctgaccctggcagatgtttgtaatcagcgcggtattcacatgacctattatggtaccggttgcatttttcattatgatgatgatttcccggtgaatagtggcaaaggttttaaagaaagcgataaaccgaattttaccggtagttattatagccataccaaagccattgttgaagatttgattaagcagtatgacaatgttctgaccctgcgcgtgcgtatgccgattattgccgatctgacctatccgcgcaattttattaccaaaattatcaaatacgacaaggttattaacatcccgaatagtatgaccgttctgccggaactgctgccgatgagtctggaaatggccaaacgcggtctgaccggtattatgaattttaccaatccgggcgcagtgagtcataatgaaattctggaaatgtataaggagtacattgatccggagtttacttggagtaattttagcgttgaagaacaggcaaaagtgattgttgccccgcgtagtaataatctgctggataccgcccgtattgaaggtgaatttccggaactgttaccgattaaggaaagtctgcgcaaatatgtgtttgaaccgaatgcagccaaaaaagatgaagtttataaggcagtgaaggaaatgcgtggtcgctaa

SEQ ID NO: 69 - protein, 289 aa, Chromochloris zofingiensis;
MATANGTSQNGHAEPVFLIFGRSGWIGGLVGELLKQQGAKFDYASARLEDRSSILAEIERVETIRSNVIGCLNLADVCLSKGLHMTYYGTGCIFHYDDEFTIESGKGFKETDKPNFTGSYYSFTKAMVESLLKEYPNVLTLRVRMPIVADLLYPRNFITKIIKYDKVINIPNSMTVLPELLPLSIKMAKRGLTGIMNYTNPGAISHNEILQLYKDYIDPDFTWKNFTVEEQAKVIVAPRSNNLLDTERIESEFPEILPIRESLIKYVFEPNAAKKDEVKAAVREMRANK

SEQ ID NO: 70 - DNA, 870 bp, Chromochloris zofingiensis;
atggccaccgcaaatggcaccagccagaatggtcatgcagaaccggtttttctgatttttggtcgtagtggctggattggcggcctggtgggtgaactgctgaaacagcagggtgccaaatttgattatgcaagtgcccgcctggaagatcgcagtagcattctggcagaaattgaacgcgttgaaaccattcgtagcaatgttattggttgtctgaatctggcagatgtgtgtctgagtaaaggtctgcacatgacctattatggcaccggctgcatttttcattatgatgatgagtttactatcgagagcggcaaaggttttaaagaaaccgataaaccgaattttaccggtagttattatagctttaccaaagccatggttgaaagcctgctgaaagaatatccgaatgttctgaccctgcgcgttcgcatgccgattgttgcagatttgctgtatccgcgcaattttattaccaaaattatcaaatacgacaaggttattaacatcccgaatagtatgaccgttctgccggaactgctgccgctgagcattaagatggcaaaacgcggtctgaccggcattatgaattataccaatccgggcgccattagtcataatgaaattctgcaactgtacaaagattacattgatccggattttacctggaaaaattttaccgttgaagaacaggccaaagtgattgtggcaccgcgcagtaataatctgctggataccgaacgcattgaaagtgaatttccggaaattctgccgattcgcgaaagtctgattaagtatgtgtttgaaccgaatgccgcaaaaaaggatgaagtgaaagcagccgttcgcgaaatgcgtgcaaataagtaa

SEQ ID NO: 71 - protein, 279 aa, Dunaliella primolecta;
MLQDMGAKFEYATARLEDRSAVLADIERVKPTHVLNAAGLTGRPNVDWCESHKVETIRANVVGCLTLADVCLTKNIHMTYYGTGCIFHYDDNFPMNSGKGFKESDQPNFTGSYYSYSKAIVESLLKEYPNVLTLRVRMPIVADLVYPRNFITKIIKYDKVVNIPNSMTVLPELLPYSIEMAKRKLTGIMNYTNPGCISHNEILELYKQYIDPEFTWQNFTLEEQAKVIVAPRSNNLLDTTRIQSEFPNILPIKESLIKYVFEPNAAKKDEVKNAVREMR

SEQ ID NO: 72 - DNA, 840 bp, Dunaliella primolecta;
atgctgcaagatatgggtgccaaatttgaatatgcaaccgcccgcctggaagatcgcagcgcagttctggcagatattgaacgtgtgaaaccgacccatgttctgaatgcagcaggcctgaccggccgtccgaatgtggattggtgcgaaagtcataaagtggaaaccattcgcgcaaatgttgtgggctgtctgaccctggccgatgtttgcctgaccaaaaatattcacatgacctattatggtaccggctgtatttttcattatgatgataatttccctatgaacagcggtaaaggttttaaagaaagcgatcagccgaattttaccggcagctattatagctatagcaaagcaattgtggaaagtctgctgaaagaatatccgaatgtgctgaccctgcgtgttcgcatgccgattgtggccgatctggtgtatccgcgtaattttattaccaaaattatcaagtacgacaaggttgtgaatattccgaatagtatgaccgttctgccggaactgctgccgtatagtattgaaatggcaaaacgtaaactgaccggtattatgaattataccaatccgggttgcattagccataatgaaattctggaactgtataaacagtacattgatccggagtttacttggcagaattttaccctggaagaacaggccaaagttattgttgcaccgcgtagcaataatctgctggataccacccgcattcagagtgaatttccgaatattctgccgattaaggaaagtctgattaagtatgtgttcgaaccgaatgccgccaaaaaagatgaagttaaaaatgcagtgcgcgaaatgcgctaa

SEQ ID NO: 73 - protein, 289 aa, Pavlova lutheri;
MNVLIFGKSGWLGGQLGELCANKGVKFQFASARLEDRAALVEEFERVKPTHILNAAGVTGRPNVDWCESHKEETLRVNVIGTMNVADVANERGIHVTLFATGCIFEYDDAHPLGSGIGFKEEDTPNFHGSFYSHTKALVEDMMRNYPNVCILRVRMPIGDDLSFHRNFIYKISKYEKVVNIPNSMTVLPEMMPISLEMARRGLTGVYNFTNPGVVSHNEILQMYKDYYDPAFTWRNFSLEEQAKVIVAARSNNELDCTKLKAEFPELLSIKDSLVKYIFEPNKGKKVAA

SEQ ID NO: 74 - DNA, 870 bp, Pavlova lutheri;
atgaacgtgctgatttttggcaaaagtggttggctgggcggtcagctgggtgaactgtgcgccaataagggtgtgaaatttcagtttgccagcgcacgtctggaagatcgcgccgcactggtggaagaatttgaacgtgtgaaaccgacccatattctgaatgcagcaggcgttaccggccgcccgaatgtggattggtgcgaaagccataaagaagaaaccctgcgtgtgaatgttattggtaccatgaatgttgcagatgtggccaatgaacgcggtattcatgttaccctgtttgccaccggctgcatttttgaatatgatgatgcacatccgctgggtagtggcattggttttaaagaagaagataccccgaattttcatggtagcttttatagtcataccaaagcactggttgaagatatgatgcgtaattatccgaatgtttgcattctgcgcgttcgcatgccgattggcgatgatctgagctttcatcgtaattttatctataagatcagcaagtacgagaaagtggtgaatattccgaatagtatgaccgttctgccggaaatgatgccgattagtctggaaatggcccgccgtggcctgaccggtgtttataattttaccaatccgggtgttgtgagccataatgaaattctgcaaatgtataaggactactatgatccggcctttacctggcgtaattttagcctggaagaacaggccaaagtgattgtggccgcccgcagcaataatgaactggattgtaccaaactgaaagcagaatttccggaactgctgagcattaaggatagtctggtgaaatatattttcgaaccgaataagggcaaaaaagttgcagcctaa

SEQ ID NO: 75 - protein, 305 aa, Nitella mirabilis;
MKALVYGRTGWIGGLLGKLCEEEGIAYEYGSGRLEDRKAIEADIVRVKPTHVFNAAGVTGRPNVDWCESHRAETIRANVIGTLNLVDVCKMHNLHVTNYATGCIFEYDDKHPEGSGIGFTEEERANFGGSFYSFSKGMVEDLLRAYDNVLTLRVRMPITSDLSNPRNFITKIARYEKVVNIPNSMTVLDELLPCAIDMARRGVTGIHNFTNPKPISHNEILELYKEYIDSDFKWTNFTLEEQAKVIVAARSNNELDATKLKAQCPHILDIKDSLIKYVFEPNRRTPKPATDAAVAAANGVARITL

SEQ ID NO: 76 - DNA, 918 bp, Nitella mirabilis;
atgaaggcactggtgtatggtcgtaccggttggattggcggtctgctgggcaaactgtgcgaagaagaaggtattgcctatgaatatggtagcggccgtctggaagatcgtaaagcaattgaagcagatattgttcgtgtgaaaccgacccatgtgtttaatgccgcaggtgttaccggccgtccgaatgtggattggtgtgaaagccatcgcgcagaaaccattcgtgccaatgttattggcaccctgaatctggtggatgtgtgtaaaatgcataatctgcatgtgaccaattatgcaaccggttgtatttttgaatacgatgataaacacccggaaggcagcggtattggctttaccgaagaagaacgcgccaattttggtggtagtttttatagttttagcaagggtatggtggaagatttgctgcgtgcctatgataatgttctgaccctgcgtgtgcgtatgccgattaccagtgatctgagcaatccgcgtaattttattaccaaaattgcccgctatgaaaaagtggttaatattccgaatagcatgaccgttctggatgaactgctgccgtgtgcaattgatatggcccgtcgtggcgttaccggtattcataattttaccaatccgaaaccgattagccataatgaaattctggaactgtataaagagtacattgatagtgatttcaagtggaccaattttaccctggaagaacaggccaaagtgattgtggccgcccgcagcaataatgaactggatgcaaccaaactgaaagcccagtgtccgcatattctggatattaaggatagcctgattaagtatgttttcgaaccgaatcgccgtaccccgaaaccggccaccgatgccgccgtggcagcagcaaatggtgtggcccgcattaccctgtaa

SEQ ID NO: 77 - protein, 298 aa, Marchantia polymorpha;
MAEANGAPAYKFLIYGKTGWIGGLLGQMCEAQGIEYVYGAGRLENRASLEDDIAGAKPTHVFNAAGVTGRPNVDWCETHKCETIRANVVGTLTLADVTRQHGLVLINYATGCIFEYDAAHPEGSGIGFKEDDTPNFIGSFYSKTKAMVEELLKNYENVCTLRVRMPITADLSNPRNFITKITRYEKVVNIPNSMTILDELLPISIEMAKRNLTGIWNFTNPGVVSHNEILEMYKEYIDPSFKYTNFNLEEQAKVIVAPRSNNELDATKLSTEFPEMLSIKESLIKNVFEPNRKTPVRN

SEQ ID NO: 78 - DNA, 897 bp, Marchantia polymorpha;
atggccgaagccaatggcgcaccggcctataaatttctgatctatggtaaaaccggttggattggtggcctgctgggtcagatgtgcgaagcccagggtattgaatatgtgtatggtgcaggtcgtctggaaaatcgcgcaagtctggaagatgatattgcaggtgccaaaccgacccatgtgtttaatgcagcaggtgtgaccggccgcccgaatgtggattggtgtgaaacccataaatgtgaaaccattcgcgcaaatgttgtgggtaccctgaccctggccgatgttacccgtcagcatggtctggttctgattaattatgccaccggctgcatttttgaatatgatgccgcacatccggaaggtagtggtattggttttaaagaagatgataccccgaattttatcggtagcttttatagcaaaaccaaagccatggtggaagaactgctgaaaaattatgaaaatgtgtgcaccctgcgcgttcgtatgccgattaccgccgatctgagcaatccgcgtaattttattaccaaaattacccgctatgagaaagtggttaatattccgaatagcatgaccattctggatgaactgctgccgattagcattgaaatggcaaaacgtaatctgaccggcatttggaattttaccaatccgggtgtggtgagtcataatgaaattctggaaatgtataaggagtacattgatccgagctttaaatataccaatttcaatctggaggagcaggccaaagtgattgttgcaccgcgcagtaataatgaactggatgccaccaaactgagcaccgaatttccggaaatgctgagcattaaggaaagcctgattaagaatgtgtttgaaccgaatcgcaaaaccccggttcgtaattaa

SEQ ID NO: 79 - protein, 308 aa, Selaginella moellendorffii;
MVVPLSSGAGNSSNGSSGGGALKFLIYGRTGWIGGLLGKLCREQGIDFVYGSGRLEDRAGLEADIAAAKPSHVMNAAGVTGRPNVDWCEDHRVETIRANVVGTLNLADVCRGHGLLLVNFATGCIFEYDGGHQIDSGVGFTEEDAPNFVGSFYSKTKAMVEELLKNYENVCTLRVRMPISSDLANPRNFITKITRYEKVVNIPNSMTVLDELLPISIEMAKRNLTGIWNFTNPGVVSHNEILEMYRQYVDPSFKWKNFSLEEQAKVIVAPRSNNELDTKKLSSEFPQLLGIKDSLVKYVFEVNSKSKK

SEQ ID NO: 80 - DNA, 927 bp, Selaginella moellendorffii;
atggttgtgccgctgagcagtggcgccggtaatagtagtaatggcagtagcggtggtggtgcactgaaatttctgatctatggtcgcaccggctggattggtggcctgctgggtaaactgtgccgtgaacagggcattgattttgtgtatggtagtggtcgcctggaagatcgtgcaggcctggaagcagatattgcagccgcaaaaccgagtcatgttatgaatgccgcaggtgtgaccggtcgtccgaatgttgattggtgtgaagatcatcgtgtggaaaccattcgtgccaatgttgtgggcaccctgaatctggccgatgtttgccgtggtcatggtctgctgctggtgaattttgccaccggttgcatttttgaatatgatggcggccatcagattgatagtggcgtgggttttaccgaagaagatgcaccgaattttgttggcagcttttatagcaaaaccaaagccatggttgaagaactgctgaaaaattatgaaaacgtttgcaccctgcgtgttcgtatgccgattagcagtgatctggcaaatccgcgtaattttattaccaaaattacccgttacgagaaagttgtgaatattccgaatagcatgaccgttctggatgaactgctgccgattagtattgaaatggcaaaacgcaatctgaccggcatttggaattttaccaatccgggcgtggttagccataatgaaattctggaaatgtatcgccagtatgttgatccgagctttaaatggaaaaattttagtctggaggagcaggccaaagttattgttgcaccgcgtagcaataatgaactggataccaaaaaactgagtagtgaatttccgcagctgctgggtattaaggatagtctggtgaaatatgttttcgaagttaatagcaagagcaaaaaataa

SEQ ID NO: 81 - protein, 298 aa, Bryum argenteum var argenteum;
MVASLNGNGEYKFLIYGKTGWIGGLLGKLCTEKGIAYEYGKGRLENRTSLEDDIAAVKPTHVFNAAGVTGRPNVDWCETHKIETIRANVVGTLTLADVCKQKDLLLINYATGCIFEYDAKHPEGSGIGFTEEEFANFTGSYYSKTKAMVEDMLREFDNVCTLRVRMPISGDLSNPRNFITKISRYNKVVNIPNSMTILDELLPISIEMAKRNLRGIWNFTNPGVVSHNEILEMYKEYIDPSFTYKNFTLEEQAKVIVAARSNNELDASKLAKEFPEMLGIKESLIKFVFEPNKKTNKA

SEQ ID NO: 82 - DNA, 897 bp, Bryum argenteum var argenteum;
atggtggccagtctgaatggcaatggcgaatataaatttctgatctatggtaaaaccggctggattggcggtctgctgggcaaactgtgtaccgaaaaaggtattgcatacgaatatggcaaaggtcgcctggaaaatcgtaccagcctggaagatgatattgccgcagttaaaccgacccatgtttttaatgccgcaggcgttaccggtcgtccgaatgttgattggtgtgaaacccataaaattgaaaccattcgcgcaaatgttgtgggtaccctgaccctggcagatgtttgtaaacagaaagatttgctgctgattaattacgccaccggctgcatttttgaatatgatgccaaacatccggaaggtagtggtattggttttaccgaagaagaatttgccaattttaccggtagttattatagcaaaaccaaagccatggtggaagatatgctgcgcgaatttgataatgtttgcaccctgcgtgtgcgtatgccgattagtggtgacctgagcaatccgcgcaattttattaccaaaattagccgctataacaaggttgtgaatattccgaatagcatgaccattctggatgaactgctgccgattagtattgaaatggcaaaacgcaatctgcgcggcatttggaattttaccaatccgggtgtggttagtcataatgaaattctggaaatgtacaaggaatacattgatccgagttttacctataaaaacttcaccctggaagaacaggccaaagtgattgttgccgcacgcagtaataatgaactggatgcaagcaaactggccaaagaatttccggaaatgctgggtattaaggaaagtctgattaagtttgttttcgaaccgaataagaaaactaataaggcataa

SEQ ID NO: 83 - protein, 746 aa, Artificial Sequence
  Synthetic polypeptide
MAANGTTPSSANEEQNKFFEDFGVWKEAPILIGSTKFEPLPDVKNIMITGGAGFIACWLVRHLTLTYPDAYNIVSFDKLDYCASLNNTRALNDKRNFSFYHGDITNPSEVVDCLERYNIDTIFHFAAQSHVDLSFGNSYAFTHTNVYGTHVLLESAKKVGIKKFIHISTDEVYGEVKDDDDDLLETSILAPTNPYAASKAAAEMLVHSYQKSFKLPVMIVRSNNVYGPHQYPEKIIPKFSCLLQRGQPVVLHGDGTPTRRYLFAGDAADAFDTILHKGTIGQIYNVGSYDEISNLTLCSKLLTYLDIPHSTQEELHKWVKHTQDRPFNDHRYAVDGTKLRQLGWDQKTSFENGMAVTVDWYKRFGERWWGDITKVLTPFPTVAGSKVVGDDNNTVEELKEEMVIDADDNMILGKKRKLNGVPSGLAQAVEAGSGTVAQNVEAVAAEPTFLIYGRNGWIGGLVGEMLKKQGAKFEYGTARLEDRAAILADIERVKPTHVLNAAGVTGRPNVDWCETHKVETIRANVIGCLNLADVCLQNGIHMTYYGTGCIFHYDDGKFKQGNGVGFQESDTPNFTGSYYSHCKAMVENLLKEFPNVLTLRVRMPIVGDLVYPRNFITKIIKYDKVVDIPNSMTVLPELLPMSIEMAKRKLTGIMNFTNPGAISHNEILELYKQYVDPEFTWSNFTLEEQAKVIVAPRSNNLMASDRIKSEFPEILSIKESLIKYVFEPAAANREETLAAVREMRGR

SEQ ID NO: 84 - DNA, 2241 bp, Artificial Sequence
  Synthetic polynucleotide
atggcagcaaatggtacaaccccgagcagcgcaaatgaagaacagaataaattctttgaggattttggcgtgtggaaagaagcaccgattctgattggtagcaccaaatttgaaccgctgccggatgttaaaaacattatgattaccggtggtgccggttttattgcatgttggctggttcgtcatctgaccctgacctatccggatgcatataacattgtgagcttcgataaactggattattgtgccagcctgaataatacccgtgcactgaatgataaacgcaactttagcttttatcacggcgatattaccaatccgagcgaagttgttgattgtctggaacgctataacatcgataccatctttcattttgcagcccagagccatgttgatctgagctttggtaatagctatgcatttacccataccaatgtttatggcacccatgttctgctggaaagcgcaaaaaaagttggcatcaaaaagttcatccacatcagcaccgatgaagtttatggtgaagtgaaagatgatgatgacgatttactggaaaccagcattctggcaccgaccaatccgtatgcagcaagcaaagcagcagcagaaatgctggtgcatagttatcagaaatcatttaaactgccggtgatgattgtgcgcagcaataatgtgtatggtccgcatcagtatccggaaaaaatcattccgaaattcagctgtctgctgcaacgtggtcagccggttgttctgcatggtgatggcaccccgacacgtcgttacctgtttgcgggtgatgcagcagatgcatttgataccattctgcataaaggcaccattggccagatttataacgttggtagctatgacgaaatcagcaatctgacactgtgtagcaaactgctgacatatctggatattccgcatagcacccaagaggaactgcataaatgggttaaacatacccaggatcgtccgtttaatgatcatcgttatgccgttgatggtacaaaactgcgtcagttaggttgggatcagaaaaccagctttgaaaatggtatggcagttaccgtggattggtataaacgttttggtgaacgttggtggggtgatattacaaaagttctgaccccgtttccgaccgttgcaggtagcaaagttgttggtgatgataataacaccgtcgaagaactgaaagaagagatggttattgacgccgatgataacatgattctgggcaaaaaacgtaaactgaatggtgttccgagcggtctggcacaggcagttgaagcaggttctggtaccgtggcacagaatgttgaagccgttgccgccgaaccgacctttctgatctatggtcgcaatggttggattggcggtctggtgggcgaaatgctgaaaaaacagggcgccaaatttgaatatggcaccgcccgtctggaagatcgtgcagccattctggccgatattgaacgtgttaaaccgacccatgtgctgaatgccgccggcgtgaccggccgtcctaatgtggattggtgcgaaacccataaagttgaaaccattcgtgcaaatgtgattggctgcctgaatctggccgatgtgtgcctgcaaaatggtattcacatgacctattatggcaccggttgcatttttcattatgatgatggtaaattcaagcagggcaatggtgtgggttttcaggaaagcgataccccgaattttaccggcagttattatagccattgtaaagcaatggtggaaaatctgctgaaagaatttccgaatgttctgaccctgcgtgtgcgcatgccgattgttggcgatctggtgtatccgcgtaattttattaccaaaattatcaagtacgacaaggtggttgatattccgaatagtatgaccgttctgccggaactgctgccgatgagcattgaaatggccaaacgcaaactgaccggcattatgaattttaccaatccgggtgcaattagccataatgaaattctggaactgtataaacagtacgttgatccggagtttacttggagcaattttaccctggaagaacaggcaaaagttattgtggccccgcgtagcaataatctgatggccagtgatcgtattaagagcgaatttccggaaattctgagcattaaggaaagtctgattaagtatgttttcgaaccggccgcagccaatcgcgaagaaaccctggccgcagttcgtgaaatgcgcggccgttaa

SEQ ID NO: 85 - protein, 736 aa, Artificial Sequence
  Synthetic polypeptide
MAANGTTPSSANEEQNKFFEDFGVWKEAPILIGSTKFEPLPDVKNIMITGGAGFIACWLVRHLTLTYPDAYNIVSFDKLDYCASLNNTRALNDKRNFSFYHGDITNPSEVVDCLERYNIDTIFHFAAQSHVDLSFGNSYAFTHTNVYGTHVLLESAKKVGIKKFIHISTDEVYGEVKDDDDDLLETSILAPTNPYAASKAAAEMLVHSYQKSFKLPVMIVRSNNVYGPHQYPEKIIPKFSCLLQRGQPVVLHGDGTPTRRYLFAGDAADAFDTILHKGTIGQIYNVGSYDEISNLTLCSKLLTYLDIPHSTQEELHKWVKHTQDRPFNDHRYAVDGTKLRQLGWDQKTSFENGMAVTVDWYKRFGERWWGDITKVLTPFPTVAGSKVVGDDNNTVEELKEEMVIDADDNMILGKKRKLNGVPSGLAQAVEAGSGAEKEPVFLVFGKSGWIGGLLGELLKEQGAKYEFASCRLEDRAAIISEIDRVKPTHVLNAAGLTGRPNVDWCETHKVETIRSNVIGCLNLADVCNQREIHMTYYGTGCIFHYDDTHPVGGEGFKEEDKPNFTGSYYSHTKAIVENLLKEFPNVLTLRVRMPIVEDLLYPRNFITKIIKYDKVVDIPNSMTVLPELLPYSIEMARRKLTGIMNFTNPGTVSHNEVLQLYKDYIDPEFTWSNFTIEEQAKVIVAPRSNNLLDTKRIESEFPMILPIKESLKKYVFEPSAEKKAELRAAVKEMRGR

SEQ ID NO: 86 - DNA, 2211 bp, Artificial Sequence
  Synthetic polynucleotide
atggcagcaaatggtacaaccccgagcagcgcaaatgaagaacagaataaattctttgaggattttggcgtgtggaaagaagcaccgattctgattggtagcaccaaatttgaaccgctgccggatgttaaaaacattatgattaccggtggtgccggttttattgcatgttggctggttcgtcatctgaccctgacctatccggatgcatataacattgtgagcttcgataaactggattattgtgccagcctgaataatacccgtgcactgaatgataaacgcaactttagcttttatcacggcgatattaccaatccgagcgaagttgttgattgtctggaacgctataacatcgataccatctttcattttgcagcccagagccatgttgatctgagctttggtaatagctatgcatttacccataccaatgtttatggcacccatgttctgctggaaagcgcaaaaaaagttggcatcaaaaagttcatccacatcagcaccgatgaagtttatggtgaagtgaaagatgatgatgacgatttactggaaaccagcattctggcaccgaccaatccgtatgcagcaagcaaagcagcagcagaaatgctggtgcatagttatcagaaatcatttaaactgccggtgatgattgtgcgcagcaataatgtgtatggtccgcatcagtatccggaaaaaatcattccgaaattcagctgtctgctgcaacgtggtcagccggttgttctgcatggtgatggcaccccgacacgtcgttacctgtttgcgggtgatgcagcagatgcatttgataccattctgcataaaggcaccattggccagatttataacgttggtagctatgacgaaatcagcaatctgacactgtgtagcaaactgctgacatatctggatattccgcatagcacccaagaggaactgcataaatgggttaaacatacccaggatcgtccgtttaatgatcatcgttatgccgttgatggtacaaaactgcgtcagttaggttgggatcagaaaaccagctttgaaaatggtatggcagttaccgtggattggtataaacgttttggtgaacgttggtggggtgatattacaaaagttctgaccccgtttccgaccgttgcaggtagcaaagttgttggtgatgataataacaccgtcgaagaactgaaagaagagatggttattgacgccgatgataacatgattctgggcaaaaaacgtaaactgaatggtgttccgagcggtctggcacaggcagttgaagcaggttctggtgcagaaaaagaaccggtgtttctggtttttggtaaaagcggctggattggcggtctgctgggcgaactgctgaaagaacagggtgccaaatatgaatttgccagttgccgcctggaagatcgtgccgccattattagtgaaattgatcgtgttaaaccgacccatgttctgaatgccgccggcctgaccggccgtcctaatgttgattggtgcgaaacccataaagttgaaaccattcgtagtaatgtgattggctgcctgaatctggccgatgtgtgtaatcagcgtgaaattcacatgacctattatggtaccggctgcatttttcattatgatgatacccatccggtgggcggtgaaggttttaaagaagaagataaaccgaatttcaccggtagctattatagtcataccaaagcaattgtggaaaatctgctgaaagagtttccgaatgtgctgaccctgcgtgtgcgtatgccgattgtggaagatttgctgtatccgcgtaattttattaccaaaattatcaagtacgacaaggttgttgatattccgaatagtatgaccgttctgccggaactgctgccgtatagcattgaaatggcccgccgtaaactgaccggcattatgaattttaccaatccgggtaccgtgagccataatgaagtgctgcaactgtataaagattatattgatccggagtttacttggagtaattttaccattgaagagcaggccaaagttattgttgcaccgcgtagtaataatctgctggataccaaacgcattgaaagtgaatttccgatgattctgccgattaaggaaagcctgaaaaaatatgttttcgaaccgagcgccgaaaagaaagccgaactgcgcgccgccgttaaagaaatgcgtggtcgttaa

SEQ ID NO: 87 - protein, 671 aa, Artificial Sequence
  Synthetic polypeptide
MASIDNGIGESEPYTPKNILITGGAGFIASHVVIRIATRYPEYKVVVLDKLDYCASVNNLSCLADKPNFRLIKGDIQSMDLISYILKTEEIDTVMHFAAQTHVDNSFGNSLAFTLNNTYGTHVLLEASRMAGTIRRFINVSTDEVYGETSLGKTTGLVESSHLDPTNPYSAAKAGAELIARAYITSYKMPVIITRGNNVYGPHQFPEKLIPKFTLLAARGKELPLHGDGSSVRSYLYVEDVAEAFDCVLHKGVTGETYNIGTDRERSVLEVARDIAKLFNLPEDKVVFVKDRAFNDRRYYIGSAKLAALGWQERTSWEEGLRKTVDWYLGLKNIENYWAGDIEMALRPHPIVVQNAITTSGAFLASGSGAEKEPVFLVFGKSGWIGGLLGELLKEQGAKYEFASCRLEDRAAIISEIDRVKPTHVLNAAGLTGRPNVDWCETHKVETIRSNVIGCLNLADVCNQREIHMTYYGTGCIFHYDDTHPVGGEGFKEEDKPNFTGSYYSHTKAIVENLLKEFPNVLTLRVRMPIVEDLLYPRNFITKIIKYDKVVDIPNSMTVLPELLPYSIEMARRKLTGIMNFTNPGTVSHNEVLQLYKDYIDPEFTWSNFTIEEQAKVIVAPRSNNLLDTKRIESEFPMILPIKESLKKYVFEPSAEKKAELRAAVKEMRGR

SEQ ID NO: 88 - DNA, 2016 bp, Artificial Sequence
  Synthetic polynucleotide
atggcaagtattgataacggtattggtgaaagtgaaccgtataccccgaaaaatattctgattaccggcggtgccggctttattgcaagccatgttgttattcgtattgccacccgttatccggaatataaagttgtggtgctggataaactggattattgcgccagtgtgaataatctgagctgcctggccgataaaccgaattttcgtctgattaagggcgatattcagagcatggatctgattagctatattctgaaaaccgaagaaatcgataccgtgatgcattttgcagcacagacccatgtggataatagttttggcaatagcctggcattcactctgaataatacctatggcacccatgttctgctggaagcaagccgcatggccggtaccattcgccgctttattaatgttagtaccgatgaagtttacggcgaaaccagtctgggcaaaaccaccggtctggttgaaagcagccatctggatccgaccaatccgtatagcgcagcaaaagcaggtgcagaactgattgcccgtgcatatattaccagttataaaatgccggttatcattacccgcggtaataatgtgtatggtccgcatcagtttccggaaaaactgattccgaaattcactctgctggcagcccgtggcaaagaactgccgctgcatggcgatggtagcagcgttcgcagctatctgtatgtggaagatgttgcagaagcctttgattgtgtgctgcataaaggtgttaccggtgaaacctataatattggcaccgatcgtgaacgcagtgtgctggaagttgcacgtgatattgcaaaactgtttaatctgccggaagataaagtggtttttgtgaaagatcgtgcattcaatgatcgtcgctattatattggtagtgcaaaactggcagcactgggctggcaggaacgcaccagttgggaagaaggcctgcgtaaaaccgttgattggtatctgggtctgaaaaatattgaaaattactgggccggcgatattgaaatggccctgcgcccgcatccgattgtggttcagaatgcaattaccaccagcggtgcctttctggccagcggttctggtgcagaaaaagaaccggtgtttctggtttttggtaaaagcggctggattggcggtctgctgggcgaactgctgaaagaacagggtgccaaatatgaatttgccagttgccgcctggaagatcgtgccgccattattagtgaaattgatcgtgttaaaccgacccatgttctgaatgccgccggcctgaccggccgtcctaatgttgattggtgcgaaacccataaagttgaaaccattcgtagtaatgtgattggctgcctgaatctggccgatgtgtgtaatcagcgtgaaattcacatgacctattatggtaccggctgcatttttcattatgatgatacccatccggtgggcggtgaaggttttaaagaagaagataaaccgaatttcaccggtagctattatagtcataccaaagcaattgtggaaaatctgctgaaagagtttccgaatgtgctgaccctgcgtgtgcgtatgccgattgtggaagatttgctgtatccgcgtaattttattaccaaaattatcaagtacgacaaggttgttgatattccgaatagtatgaccgttctgccggaactgctgccgtatagcattgaaatggcccgccgtaaactgaccggcattatgaattttaccaatccgggtaccgtgagccataatgaagtgctgcaactgtataaagattatattgatccggagtttacttggagtaattttaccattgaagagcaggccaaagttattgttgcaccgcgtagtaataatctgctggataccaaacgcattgaaagtgaatttccgatgattctgccgattaaggaaagcctgaaaaaatatgttttcgaaccgagcgccgaaaagaaagccgaactgcgcgccgccgttaaagaaatgcgtggtcgttaa

SEQ ID NO: 89 - protein, 343 aa, Tetraselmis cordiformis;
MGEEKPYIPTSILVTGGAGFIGSHVTLRLLQNYDYKVVVLDKMDYCASLKNLESVKDKPNFKFIKGDIQSADLLNYILEAEKIDTIMHFAAQTHVDNSFGNSLAFTMNNTFGTHVLLESARCYGKIRRFINVSTDEVYGETSLGSEHGLDESSKMEPTNPYSAAKAGAEMLAQAYITSYKMPIIITRGNNVYGPHQFPEKMIPKFTLLASRGQELPIHGDGMARRSYLYVEDVARAFDCVLHKGETGETYNIGTQKERTVLEVAQAIAKIFKLDGEKVQHVRDRAFNDRRYYICDQKLNKMGWHEEVEFEEGLKKTVEWYLYNGFSNYWDDAEVELALRAHPL

SEQ ID NO: 90 - DNA, 1032 bp, Tetraselmis cordiformis;
atgggtgaagaaaaaccgtatattccgaccagcattctggtgaccggcggtgcaggttttattggcagccatgtgaccctgcgtctgctgcaaaattatgattataaagtggttgtgctggataaaatggattattgtgccagcctgaaaaatctggaaagcgtgaaagataaaccgaattttaaattcatcaagggcgatattcagagcgccgatctgctgaattatattctggaagccgaaaaaattgacaccattatgcattttgccgcccagacccatgttgataatagctttggcaatagtctggcctttaccatgaataatacctttggtacccatgttctgctggaaagcgcacgctgttatggcaaaattcgccgttttattaatgttagtaccgatgaagtttacggcgaaaccagcctgggcagtgaacatggcctggatgaaagtagcaaaatggaaccgaccaatccgtatagcgcagcaaaagcaggtgccgaaatgctggcccaggcatatattaccagctataaaatgccgattatcattacccgtggtaataatgtttacggcccgcatcagtttccggaaaaaatgattccgaaattcactctgctggcaagtcgtggtcaggaactgccgattcatggtgacggtatggcacgtcgcagttatctgtatgttgaagatgtggcccgcgcctttgattgcgtgctgcataaaggtgaaaccggcgaaacctataatattggcacccagaaagaacgtaccgttctggaagttgcacaggcaattgccaaaatttttaaactggatggtgaaaaagtgcagcatgttcgcgatcgcgcctttaatgatcgtcgttattatatttgcgaccagaaactgaataagatgggttggcatgaagaagtggaatttgaagaaggtctgaaaaagactgtggaatggtatctgtataatggctttagtaattactgggatgatgcagaagtggaactggccctgcgcgcacatccgctgtaa

SEQ ID NO: 91 - protein, 296 aa, Arabidopsis thaliana;
QRSNGTPQKPSLKFLIYGKTGWIGGLLGKICDKQGIAYEYGKGRLEDRSSLLQDIQSVKPTHVFNSAGVTGRPNVDWCESHKTETIRANVAGTLTLADVCREHGLLMMNFATGCIFEYDDKHPEGSGIGFKEEDTPNFTGSFYSKTKAMVEELLKEYDNVCTLRVRMPISSDLNNPRNFITKISRYNKVVNIPNSMTVLDELLPISIEMAKRNLKGIWNFTNPGVVSHNEILEMYRDYINPEFKWANFTLEEQAKVIVAPRSNNEMDASKLKKEFPELLSIKESLIKYAYGPNKKT

SEQ ID NO: 92 - DNA, 891 bp, Arabidopsis thaliana;
cagcgtagcaatggtacaccgcagaaaccgagcctgaaatttctgatttatggtaaaaccggttggattggtggtctgctgggtaaaatttgcgataaacagggtatcgcctatgaatatggtaaaggtcgtctggaagatcgtagcagcctgctgcaagatattcagagcgttaaaccgacgcatgtgtttaatagtgccggtgtgaccggtcgtccgaatgttgattggtgtgaaagccataaaaccgaaaccattcgtgcaaatgttgcaggtacactgaccctggcagatgtttgtcgtgaacatggtttactgatgatgaattttgccaccggctgcatctttgagtatgatgataaacatccggaaggtagcggtatcggttttaaagaagaagatacaccgaattttaccggcagcttttacagcaaaaccaaagcaatggttgaggaactgctgaaagaatatgataatgtttgtaccctgcgtgtgcgtatgccgattagcagcgacctgaataatccgcgtaactttattaccaaaatctcccgctataacaaagtggtgaatattccgaatagcatgaccgtactggatgaactgctgcctattagcattgaaatggcaaaacgtaacctgaaaggcatctggaactttaccaatccgggtgttgttagccataacgaaattctggaaatgtaccgcgattatatcaacccggaatttaagtgggccaattttacactggaagaacaggccaaagttattgttgcaccgcgtagtaataatgaaatggatgcaagcaaactgaagaaagagtttccagaactgctgtccattaaagaaagcctgatcaaatatgcgtacggtccgaacaaaaaaacctaa

SEQ ID NO: 93 - protein, 291 aa, Pyricularia oryzae;
TNNRFLIWGGEGWVAGHLASILKSQGKDVYTTTVRMENREGVLAELEKVKPTHVLNCAGCTGRPNVDWCEDNKEATMRSNVIGTLNLTDACFQKGIHCTVFATGCIYQYDDAHPWDGPGFLETDKANFAGSFYSETKAHVEEVMKYYNNCLILRLRMPVSDDLHPRNFVTKIAKYDRVVDIPNSNTILHDLLPLSLAMAEHKDTGVYNFTNPGAISHNEVLTLFRDIVRPSFKWQNFSLEEQAKVIKAGRSNCKLDTTKLTEKAKEYGIEVPEIHEAYRQCFERMKKAGVQ

SEQ ID NO: 94 - DNA, 876 bp, Pyricularia oryzae;
accaataaccgttttctgatttggggtggtgaaggttgggttgcaggtcatctggcaagcattctgaaaagccagggtaaagatgtttataccaccaccgttcgtatggaaaatcgtgaaggtgttctggcagaactggaaaaagttaaaccgacacatgttctgaattgtgcaggttgtaccggtcgtccgaatgttgattggtgtgaagataataaagaagccaccatgcgtagcaatgttattggcaccctgaatctgaccgatgcatgttttcagaaaggtattcattgtaccgtttttgccaccggttgcatctatcagtatgatgatgcacatccgtgggatggtccgggttttctggaaaccgataaagcaaattttgccggtagcttttacagcgaaaccaaagcacatgttgaagaggtgatgaagtattacaacaactgtctgattctgcgtctgcgtatgccggttagtgatgatctgcatccgcgtaattttgtgaccaaaatcgcaaaatatgatcgcgttgtggatattccgaatagcaataccattctgcatgatctgctgccgctgagcctggcaatggcagaacataaagataccggtgtttacaactttaccaatccgggtgcaattagccataatgaagttctgaccctgtttcgtgatattgttcgtccgagctttaagtggcagaatttttcactggaagaacaggccaaagttattaaagcaggtcgtagcaattgtaaactggataccaccaaactgaccgaaaaagccaaagaatatggtattgaagtgccggaaattcatgaagcatatcgtcagtgttttgaacgcatgaaaaaagccggtgttcagtaa

SEQ ID NO: 95 - protein, 295 aa, Citrus clementina;
SKCSSPRKPSMKFLIYGRTGWIGGLLGKLCEKEGIPFEYGKGRLEDRSSLIADVQSVKPTHVFNAAGVTGRPNVDWCESHKTDTIRTNVAGTLTLADVCREHGILMMNYATGCIFEYDAAHPEGSGIGYKEEDTPNFTGSFYSKTKAMVEELLKEYDNVCTLRVRMPISSDLNNPRNFITKISRYNKVVNIPNSMTVLDELLPISIEMAKRNLRGIWNFTNPGVVSHNEILEMYKKYINPEFKWVNFTLEEQAKVIVAPRSNNEMDASKLKKEFPELLSIKDSLIKYVFEPNKKT

SEQ ID NO: 96 - DNA, 876 bp, Citrus clementina;; identical to SEQ ID NO: 94

SEQ ID NO: 97 - protein, 462 aa, Oryza sativa;
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 98 - DNA, 1389 bp, Oryza sativa;
atggattcgggttactcttcctcctatgcggcggctgcgggtatgcacgttgttatctgtccgtggctggcttttggtcacctgctgccgtgcctggatctggcacagcgtctggcttcacgcggccatcgtgtcagcttcgtgtctaccccgcgcaatatttcgcgtctgccgccggttcgtccggcactggctccgctggttgcatttgtcgctctgccgctgccgcgcgtggaaggtctgccggatggtgcggaaagtaccaacgacgtgccgcatgatcgcccggacatggttgaactgcaccgtcgtgcattcgatggtctggcagcaccgttttccgaatttctgggtacggcgtgcgccgattgggtgatcgttgacgtctttcatcactgggcggcggcggcggcgctggaacataaagttccgtgtgcaatgatgctgctgggctcagctcacatgattgcgtcgatcgcagaccgtcgcctggaacgtgcagaaaccgaaagtccggctgcggccggccagggtcgcccggcagctgcgccgaccttcgaagtggcccgcatgaaactgattcgtacgaaaggcagctctggtatgagcctggcagaacgctttagtctgaccctgtcccgtagttccctggtggttggtcgcagttgcgttgaatttgaaccggaaaccgtcccgctgctgtccacgctgcgtggtaaaccgatcacctttctgggtctgatgccgccgctgcatgaaggccgtcgcgaagatggtgaagacgcaacggtgcgttggctggatgcacagccggctaaaagcgtcgtgtatgtcgccctgggctctgaagtgccgctgggtgtggaaaaagttcacgaactggcactgggcctggaactggctggcacccgcttcctgtgggcactgcgtaaaccgacgggtgtgagcgatgcggacctgctgccggccggttttgaagaacgtacccgcggccgtggtgttgtcgcaacgcgttgggtcccgcaaatgagcattctggcgcatgccgcagtgggcgcctttctgacccactgtggttggaacagcacgatcgaaggcctgatgtttggtcacccgctgattatgctgccgatcttcggcgatcagggtccgaacgcacgtctgattgaagcgaaaaatgccggcctgcaagttgcgcgcaacgatggcgacggttctttcgaccgtgagggtgtggctgcggccattcgcgcagtggctgttgaagaagaatcatcgaaagtttttcaggcgaaagccaaaaaactgcaagaaatcgtcgcggatatggcctgccacgaacgctacattgatggtttcattcagcaactgcgctcctacaaagactaa

SEQ ID NO: 99 - protein, 459 aa, Hordeum vulgare;
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 100 - DNA, 1380 bp, Hordeum vulgare;
atggatggtaactcctcctcctcgccgctgcatgtggtcatttgtccgtggctggctctgggtcacctgctgccgtgtctggatattgctgaacgtctggcgtcacgcggccatcgtgtcagttttgtgtccaccccgcgcaacattgcccgtctgccgccgctgcgtccggctgttgcaccgctggttgatttcgtcgcactgccgctgccgcatgttgacggtctgccggagggtgcggaatcgaccaatgatgtgccgtatgacaaatttgaactgcaccgtaaggcgttcgatggtctggcggccccgtttagcgaatttctgcgtgcagcttgcgcagaaggtgcaggttctcgcccggattggctgattgtggacacctttcatcactgggcggcggcggcggcggtggaaaacaaagtgccgtgtgttatgctgctgctgggtgcagcaacggtgatcgctggtttcgcgcgtggtgttagcgaacatgcggcggcggcggtgggtaaagaacgtccggctgcggaagccccgagttttgaaaccgaacgtcgcaagctgatgaccacgcagaatgcctccggcatgaccgtggcagaacgctatttcctgacgctgatgcgtagcgatctggttgccatccgctcttgcgcagaatgggaaccggaaagcgtggcagcactgaccacgctggcaggtaaaccggtggttccgctgggtctgctgccgccgagtccggaaggcggtcgtggcgtttccaaagaagatgctgcggtccgttggctggacgcacagccggcaaagtcagtcgtgtacgtcgcactgggttcggaagtgccgctgcgtgcggaacaagttcacgaactggcactgggcctggaactgagcggtgctcgctttctgtgggcgctgcgtaaaccgaccgatgcaccggacgccgcagtgctgccgccgggtttcgaagaacgtacccgcggccgtggtctggttgtcacgggttgggtgccgcagattggcgttctggctcatggtgcggtggctgcgtttctgacccactgtggctggaactctacgatcgaaggcctgctgttcggtcatccgctgattatgctgccgatcagctctgatcagggtccgaatgcgcgcctgatggaaggccgtaaagtcggtatgcaagtgccgcgtgatgaatcagacggctcgtttcgtcgcgaagatgttgccgcaaccgtccgcgccgtggcagttgaagaagacggtcgtcgcgtcttcacggctaacgcgaaaaagatgcaagaaattgtggccgatggcgcatgccacgaacgttgtattgacggttttatccagcaactgcgcagttacaaggcgtaa

SEQ ID NO: 101 - protein, 473 aa, Artificial Sequence
  Synthetic polypeptide
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 102 - DNA, 1422 bp, Artificial Sequence
  Synthetic polynucleotide
atggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatggataccgtatggggatggttcttaagggatctgattgtttgctttccaaatgttaccatgagtttggaactcaatggctacctcttttggagacactacaccaagtaccggtggttccggtgggattactgccaccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatgcgcgtgcggttgccatcgatcatgagagttaa

SEQ ID NO: 103 - protein, 464 aa, Oryza brachyantha;
MENGSSPLHVVIFPWLAFGHLLPFLDLAERLAARGHRVSFVSTPRNLARLRPVRPALRGLVDLVALPLPRVHGLPDGAEATSDVPFEKFELHRKAFDGLAAPFSAFLDAACAGDKRPDWVIPDFMHYWVAAAAQKRGVPCAVLIPCSADVMALYGQPTETSTEQPEAIARSMAAEAPSFEAERNTEEYGTAGASGVSIMTRFSLTLKWSKLVALRSCPELEPGVFTTLTRVYSKPVVPFGLLPPRRDGAHGVRKNGEDDGAIIRWLDEQPAKSVVYVALGSEAPVSADLLRELAHGLELAGTRFLWALRRPAGVNDGDSILPNGFLERTGERGLVTTGWVPQVSILAHAAVCAFLTHCGWGSVVEGLQFGHPLIMLPIIGDQGPNARFLEGRKVGVAVPRNHADGSFDRSGVAGAVRAVAVEEEGKAFAANARKLQEIVADRERDERCTDGFIHHLTSWNELEA

SEQ ID NO: 104 - DNA, 1395 bp, Oryza brachyantha;
atggaaaatggtagcagtccgctgcatgttgttatttttccgtggctggcatttggtcatctgctgccgtttctggatctggcagaacgtctggcagcacgtggtcatcgtgttagctttgttagcacaccgcgtaatctggcacgtctgcgtccggttcgtccggcactgcgtggtctggttgatctggttgcactgccgctgcctcgtgttcatggtctgccggatggtgccgaagcaaccagtgatgttccgtttgaaaaatttgaactgcaccgcaaagcatttgatggcctggctgcaccgtttagcgcatttctggatgcagcatgtgccggtgataaacgtccggattgggttattccggattttatgcattattgggttgcagcagcagcacagaaacgtggtgttccgtgtgcagttctgattccgtgtagcgcagatgttatggcactgtatggtcagccgaccgaaaccagcaccgaacagccggaagcaattgcacgtagcatggcagcagaagcaccgagctttgaagcagaacgtaataccgaagaatatggtacagccggtgcaagcggtgttagcattatgacccgttttagtctgaccctgaaatggtcaaaactggttgccctgcgtagctgtccggaactggaaccgggtgtttttaccacactgacccgtgtttatagcaaaccggttgtgccgtttggtctgctgcctccgcgtcgtgatggtgcacatggtgttcgtaaaaatggtgaagatgatggtgccattattcgttggctggatgaacagcctgcaaaaagcgttgtttatgttgcactgggtagcgaagcaccggtttcagccgatctgctgcgtgaactggcacatggtctggaattagcaggcacccgttttctgtgggctctgcgtcgtcctgccggtgttaatgatggtgatagcattctgccgaatggttttctggaacgtaccggtgaacgcggtctggttaccaccggttgggttccgcaggttagtattctggcccatgcagcagtttgtgcatttctgacccattgtggttggggtagcgttgttgaaggtttacagtttggccatccgctgattatgctgccgattattggtgatcagggtccgaatgcacgctttctggaaggtcgtaaagttggtgttgcagttccgcgtaaccatgcagatggtagctttgatcgtagcggtgttgccggtgccgttcgtgcagttgcagttgaagaagaaggtaaagcctttgcagcaaatgcccgtaaactgcaagaaattgttgcagatcgtgaacgtgatgaacgttgtaccgatggttttattcatcatctgaccagctggaatgaactggaagcataa

SEQ ID NO: 105 - protein, 475 aa, Artificial Sequence
  Synthetic polypeptide
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLYKDDSGYSSSYAAAAGMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 106 - DNA, 1428 bp, Artificial Sequence
  Synthetic polynucleotide
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctgtacaaagatgacagcggttatagcagcagctatgcggcggcggcgggtatggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 107 - protein, 458 aa, Stevia rebaudiana;
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 108 - DNA, 1377 bp, Stevia rebaudiana;
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 109 - protein, 1268 aa, Artificial Sequence
  Synthetic polypeptide
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 110 - DNA, 3807 bp, Artificial Sequence
  Synthetic polynucleodie
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga